SEQUENCE LISTING (164 sequences)

SEQ ID NO: 1 - protein, 281 aa, Artificial Sequence
  Description of Artificial Sequence Synthetic polypeptide
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 2 - DNA, 780 bp, Artificial Sequence
  Description of Artificial Sequence Synthetic polynucleotide
gaattcaccggtgccgccaccatgaagtgggtgaccttcatcagcctgctgttcctgttcagcagcgcctacagctggagccacccccagttcgagaagggcagcggcgacgacgacgacaagggcagcggcggcaagatcgccgccctgaagcagaagatcgccgccctgaagtacaagaacgccgccctgaagaagaagatcgccgccctgaagcagggcggcggatcccagctgttccacctgcagaaggagctggccgagctgcgcgagagcaccagccagatgcacaccgccagcagcctggagaagcagatcggccaccccagccccccccccgagaagaaggagctgcgcaaggtggcccacctgaccggcaagagcaacagccgcagcatgcccctggagtgggaggacacctacggcatcgtgctgctgagcggcgtgaagtacaagaagggcggcctggtgatcaacgagaccggcctgtacttcgtgtacagcaaggtgtacttccgcggccagagctgcaacaacctgcccctgagccacaaggtgtacatgcgcaacagcaagtacccccaggacctggtgatgatggagggcaagatgatgagctactgcaccaccggccagatgtgggcccgcagcagctacctgggcgccgtgttcaacctgaccagcgccgaccacctgtacgtgaacgtgagcgagctgagcctggtgaacttcgaggagagccagaccttcttcggcctgtacaagctgtgatgagctagc

SEQ ID NO: 3 - protein, 281 aa, Artificial Sequence [sequence omitted]

SEQ ID NO: 4 - DNA, 969 bp, Artificial Sequence
  Description of Artificial Sequence Synthetic polynucleotide
gaattcaccggtgccgccaccatgaagtgggtgaccttcatcagcctgctgttcctgttcagcagcgcctacagctggagccacccccagttcgagaagggcagcggcgacgacgacgacaagggcagcggcggcaagatcgccgccctgaagcagaagatcgccgccctgaagtacaagaacgccgccctgaagaagaagatcgccgccctgaagcagggcggcggatccaacgagctgaagcagatgcaggacaagtacagcaagagcggcatcgcctgcttcctgaaggaggacgacagctactgggaccccaacgacgaggagagcatgaacagcccctgctggcaggtgaagtggcagctgcgccagctggtgcgcaagatgatcctgcgcaccagcgaggagaccatcagcaccgtgcaggagaagcagcagaacatcagccccctggtgcgcgagcgcggcccccagcgcgtggccgcccacatcaccggcacccgcggccgcagcaacaccctgagcagccccaacagcaagaacgagaaggccctgggccgcaagatcaacagctgggagagcagccgcagcggccacagcttcctgagcaacctgcacctgcgcaacggcgagctggtgatccacgagaagggcttctactacatctacagccagacctacttccgcttccaggaggagatcaaggagaacaccaagaacgacaagcagatggtgcagtacatctacaagtacaccagctaccccgaccccatcctgctgatgaagagcgcccgcaacagctgctggagcaaggacgccgagtacggcctgtacagcatctaccagggcggcatcttcgagctgaaggagaacgaccgcatcttcgtgagcgtgaccaacgagcacctgatcgacatggaccacgaggccagcttcttcggcgccttcctggtgggctgatgagctagc

SEQ ID NO: 5 - protein, 249 aa, Artificial Sequence
  Description of Artificial Sequence Synthetic polypeptide
MAARRSQRRRGRRGEPGTALLVPLALGLGLALACLGLLLAVVSLGSRASLSAQEPAQEELVAEEDQDPSELNPQTEESQDPAPFLNRLVRPRRSAPKGRKTRARRAIAAHYEVHPRPGQDGAQAGVDGTVSGWEEARINSSSPLRYNRQIGEFIVTRAGLYYLYCQVHFDEGKAVYLKLDLLVDGVLALRCLEEFSATAASSLGPQLRLCQVSGLLALRPGSSLRIRTLPWAHLKAAPFLTYFGLFQVH

SEQ ID NO: 6 - protein, 183 aa, Artificial Sequence
  Description of Artificial Sequence Synthetic polypeptide
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 7 - protein, 233 aa, Artificial Sequence
  Description of Artificial Sequence Synthetic polypeptide
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 8 - protein, 244 aa, Artificial Sequence
  Description of Artificial Sequence Synthetic polypeptide
MGALGLEGRGGRLQGRGSLLLAVAGATSLVTLLLAVPITVLAVLALVPQDQGGLVTETADPGAQAQQGLGFQKLPEEEPETDLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 9 - protein, 205 aa, Artificial Sequence
  Description of Artificial Sequence Synthetic polypeptide
MTPPERLFLPRVCGTTLHLLLLGLLLVLLPGAQGLPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 10 - protein, 261 aa, Artificial Sequence
  Description of Artificial Sequence Synthetic polypeptide
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 11 - protein, 240 aa, Artificial Sequence
  Description of Artificial Sequence Synthetic polypeptide
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 12 - protein, 193 aa, Artificial Sequence
  Description of Artificial Sequence Synthetic polypeptide
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 13 - protein, 234 aa, Artificial Sequence
  Description of Artificial Sequence Synthetic polypeptide
MDPGLQQALNGMAPPGDTAMHVPAGSVASHLGTTSRSYFYLTTATLALCLVFTVATIMVLVVQRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 14 - protein, 254 aa, Artificial Sequence
  Description of Artificial Sequence Synthetic polypeptide
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 15 - protein, 317 aa, Artificial Sequence
  Description of Artificial Sequence Synthetic polypeptide
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 16 - protein, 250 aa, Artificial Sequence [sequence omitted]

SEQ ID NO: 17 - protein, 241 aa, Artificial Sequence
  Description of Artificial Sequence Synthetic polypeptide
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 18 - protein, 247 aa, Artificial Sequence
  Description of Artificial Sequence Synthetic polypeptide
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 19 - protein, 257 aa, Artificial Sequence
  Description of Artificial Sequence Synthetic polypeptide
MSILFYVIFLAYLRGIQGNNMDQRSLPEDSLNSLIIKLIQADILKNKLSKQMVDVKENYQSTLPKAEAPREPERGGPAKSAFQPVIAMDTELLRQQRRYNSPRVLLSDSTPLEPPPLYLMEDYVGSPVVANRTSRRKRYAEHKSHRGEYSVCDSESLWVTDKSSAIDIRGHQVTVLGEIKTGNSPVKQYFYETRCKEARPVKNGCRGIDDKHWNSQCKTSQTYVRALTSENNKLVGWRWIRIDTSCVCALSRKIGRT

SEQ ID NO: 20 - protein, 210 aa, Artificial Sequence
  Description of Artificial Sequence Synthetic polypeptide
MLPLPSCSLPILLLFLLPSVPIESQPPPSTLPPFLAPEWDLLSPRVVLSRGAPAGPPLLFLLEAGAFRESAGAPANRSRRGVSETAPASRRGELAVCDAVSGWVTDRRTAVDLRGREVEVLGEVPAAGGSPLRQYFFETRCKADNAEEGGPGAGGGGCRGVDRRHWVSECKAKQSYVRALTADAQGRVGWRWIRIDTACVCTLLSRTGRA

SEQ ID NO: 21 - protein, 285 aa, Artificial Sequence
  Description of Artificial Sequence Synthetic polypeptide
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 22 - protein, 199 aa, Artificial Sequence
  Description of Artificial Sequence Synthetic polypeptide
MTLHPSPITCEFLFSTALISPKMCLSHLENMPLSHSRTQGAQRSSWKLWLFCSIVMLLFLCSFSWLIFIFLQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 23 - protein, 251 aa, Artificial Sequence
  Description of Artificial Sequence Synthetic polypeptide
MAEDLGLSFGETASVEMLPEHGSCRPKARSSSARWALTCCLVLLPFLAGLTTYLLVSQLRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 24 - protein, 391 aa, Artificial Sequence
  Description of Artificial Sequence Synthetic polypeptide
MGYPEVERRELLPAAAPRERGSQGCGCGGAPARAGEGNSCLLFLGFFGLSLALHLLTLCCYLELRSELRRERGAESRLGGSGTPGTSGTLSSLGGLDPDSPITSHLGQPSPKQQPLEPGEAALHSDSQDGHQMALLNFFFPDEKPYSEEESRRVRRNKRSKSNEGADGPVKNKKKGKKAGPPGPNGPPGPPGPPGPQGPPGIPGIPGIPGTTVMGPPGPPGPPGPQGPPGLQGPSGAADKAGTRENQPAVVHLQGQGSAIQVKNDLSGGVLNDWSRITMNPKVFKLHPRSGELEVLVDGTYFIYSQVEVYYINFTDFASYEVVVDEKPFLQCTRSIETGKTNYNTCYTAGVCLLKARQKIAVKMVHADISINMSKHTTFFGAIRLGEAPAS

SEQ ID NO: 25 - protein, 383 aa, Artificial Sequence
  Description of Artificial Sequence Synthetic polypeptide
MGLGPVFLLLAGIFPFAPPGAAAEPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHWQTFHVSAVAAAAIFVIIIFYVRCCKKKTSAAEGPELVSLQVLDQHPVGTSDHRDATQLGFQPLMSDLGSTGSTEGA

SEQ ID NO: 26 - protein, 383 aa, Artificial Sequence [sequence omitted]

SEQ ID NO: 27 - protein, 244 aa, Artificial Sequence
  Description of Artificial Sequence Synthetic polypeptide
MAAAASPAFLLCLPLLHLLSGWSRAGWVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAPGTTQPKAMATTLSPWSLLIIFLCFILAGR

SEQ ID NO: 28 - protein, 246 aa, Artificial Sequence
  Description of Artificial Sequence Synthetic polypeptide
MAAAAATKILLCLPLLLLLSGWSRAGRADPHSLCYDITVIPKFRPGPRWCAVQGQVDEKTFLHYDCGNKTVTPVSPLGKKLNVTTAWKAQNPVLREVVDILTEQLRDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPSAGAPLAMSSGTTQLRATATTLILCCLLIILPCFILPGI

SEQ ID NO: 29 - protein, 244 aa, Artificial Sequence
  Description of Artificial Sequence Synthetic polypeptide
MAAAASPAILPRLAILPYLLFDWSGTGRADAHSLWYNFTIIHLPRHGQQWCEVQSQVDQKNFLSYDCGSDKVLSMGHLEEQLYATDAWGKQLEMLREVGQRLRLELADTELEDFTPSGPLTLQVRMSCECEADGYIRGSWQFSFDGRKFLLFDSNNRKWTVVHAGARRMKEKWEKDSGLTTFFKMVSMRDCKSWLRDFLMHRKKRLEPTAPPTMAPGLAQPKAIATTLSPWSFLIILCFILPGI

SEQ ID NO: 30 - protein, 263 aa, Artificial Sequence
  Description of Artificial Sequence Synthetic polypeptide
MRRISLTSSPVRLLLFLLLLLIALEIMVGGHSLCFNFTIKSLSRPGQPWCEAQVFLNKNLFLQYNSDNNMVKPLGLLGKKVYATSTWGELTQTLGEVGRDLRMLLCDIKPQIKTSDPSTLQVEMFCQREAERCTGASWQFATNGEKSLLFDAMNMTWTVINHEASKIKETWKKDRGLEKYFRKLSKGDCDHWLREFLGHWEAMPEPTVSPVNASDIHWSSSSLPDRWIILGAFILLVLMGIVLICVWWQNGEWQAGLWPLRTS

SEQ ID NO: 31 - protein, 334 aa, Artificial Sequence
  Description of Artificial Sequence Synthetic polypeptide
MAAAASPAFLLRLPLLLLLSSWCRTGLADPHSLCYDITVIPKFRPGPRWCAVQGQVDEKTFLHYDCGSKTVTPVSPLGKKLNVTTAWKAQNPVLREVVDILTEQLLDIQLENYIPKEPLTLQARMSCEQKAEGHGSGSWQLSFDGQIFLLFDSENRMWTTVHPGARKMKEKWENDKDMTMSFHYISMGDCTGWLEDFLMGMDSTLEPSAGAPPTMSSGTAQPRATATTLILCCLLIMCLLICSRHSLTQSHGHHPQSLQPPPHPPLLHPTWLLRRVLWSDSYQIAKRPLSGGHVTRVTLPIIGDDSHSLPCPLALYTINNGAARYSEPLQVSIS

SEQ ID NO: 32 - protein, 246 aa, Artificial Sequence
  Description of Artificial Sequence Synthetic polypeptide
MAAAAIPALLLCLPLLFLLFGWSRARRDDPHSLCYDITVIPKFRPGPRWCAVQGQVDEKTFLHYDCGNKTVTPVSPLGKKLNVTMAWKAQNPVLREVVDILTEQLLDIQLENYTPKEPLTLQARMSCEQKAEGHSSGSWQFSIDGQTFLLFDSEKRMWTTVHPGARKMKEKWENDKDVAMSFHYISMGDCIGWLEDFLMGMDSTLEPSAGAPLAMSSGTTQLRATATTLILCCLLIILPCFILPGI

SEQ ID NO: 33 - protein, 253 aa, Artificial Sequence
  Description of Artificial Sequence Synthetic polypeptide
MAKAAVTKRHHFMIQKLLILLSYGYTNGLDDAHSLRCNLTIKDPTPADPLWYEAKCLVDEILILHLSNINKTMTSGDPGETANATEVGECLTQPLKDLCQKLRNKVSNTKVDTHKTNGYPHLQVTMIYLQSQGQIPSATWEFNISDSYFFTFYTENMSWRSANDESGVIMNKWKDDGEFVKRLKFLIPECRQEVDEFLKQPKEKPRSTSRSPSITQLTSTSPLPPPSHSTSKKGFISVGLIFISLLFAFAFAM

SEQ ID NO: 34 - protein, 232 aa, Artificial Sequence
  Description of Artificial Sequence Synthetic polypeptide
MALIRDRKSHHSEMSKCHNYDLKPAKWDTSQEQQKQRLALTTSQPGENGIIRGRYPIEKLKISPMFVVRVLAIALAIRFTLNTLMWLAIFKETFQPVLCNKEVPVSSREGYCGPCPNNWICHRNNCYQFFNEEKTWNQSQASCLSQNSSLLKIYSKEEQDFLKLVKSYHWMGLVQIPANGSWQWEDGSSLSYNQLTLVEIPKGSCAVYGSSFKAYTEDCANLNTYICMKRAV

SEQ ID NO: 35 - protein, 244 aa, Artificial Sequence
  Description of Artificial Sequence Synthetic polypeptide
MAKGATSKSNHCLILSLFILLSYLGTILADGTDSLSCELTFNYRNLHGQCSVNGKTLLDFGDKKHEENATKMCADLSQNLREISEEMWKLQSGNDTLNVTTQSQYNQGKFIDGFWAINTDEQHSIYFYPLNMTWRESHSDNSSAMEQWKNKNLEKDMRNFLITYFSHCLNKSSSHFREMPKSTLKVPDTTQRTNATQIHPTVNNFRHNSDNQGLSVTWIVIICIGGLVSFMAFMVFAWCMLKKK

SEQ ID NO: 36 - protein, 417 aa, Artificial Sequence
  Description of Artificial Sequence Synthetic polypeptide
MARAMAAAWPLLLVALLVLSWPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRNAIIFLVLGILVFLILLGIGIYFYWSKCSREVLWHCHLCPSSTEHASASANGHVSYSAVSRENSSSQDPQTEGTR

SEQ ID NO: 37 - protein, 538 aa, Artificial Sequence
  Description of Artificial Sequence Synthetic polypeptide
MARAAALLPSRSPPTPLLWPLLLLLLLETGAQDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIFVRETPNTAGAGATGGIIGGIIAAIIATAVAATGILICRQQRKEQTLQGAEEDEDLEGPPSYKPPTPKAKLEAQEMPSQLFTLGASEHSPLKTPYFDAGASCTEQEMPRYHELPTLEERSGPLHPGATSLGSPIPVPPGPPAVEDVSLDLEDEEGEEEEEYLDKINPIYDALSYSSPSDSYQGKGFVMSRAMYV

SEQ ID NO: 38 - protein, 454 aa, Artificial Sequence
  Description of Artificial Sequence Synthetic polypeptide
MTWRAAASTCAALLILLWALTTEGDLKVEMMAGGTQITPLNDNVTIFCNIFYSQPLNITSMGITWFWKSLTFDKEVKVFEFFGDHQEAFRPGAIVSPWRLKSGDASLRLPGIQLEEAGEYRCEVVVTPLKAQGTVQLEVVASPASRLLLDQVGMKENEDKYMCESSGFYPEAINITWEKQTQKFPHPIEISEDVITGPTIKNMDGTFNVTSCLKLNSSQEDPGTVYQCVVRHASLHTPLRSNFTLTAARHSLSETEKTDNFSIHWWPISFIGVGLVLLIVLIPWKKICNKSSSAYTPLKCILKHWNSFDTQTLKKEHLIFFCTRAWPSYQLQDGEAWPPEGSVNINTIQQLDVFCRQEGKWSEVPYVQAFFALRDNPDLCQCCRIDPALLTVTSGKSIDDNSTKSEKQTPREHSDAVPDAPILPVSPIWEPPPATTSTTPVLSSQPPTLLLPLQ

SEQ ID NO: 39 - protein, 1132 aa, Artificial Sequence
  Description of Artificial Sequence Synthetic polypeptide
MEPNDSTSTAVEEPDSLEVLVKTLDSQTRTFIVGAQMNVKEFKEHIAASVSIPSEKQRLIYQGRVLQDDKKLQEYNVGGKVIHLVERAPPQTHLPSGASSGTGSASATHGGGSPPGTRGPGASVHDRNANSYVMVGTFNLPSDGSAVDVHINMEQAPIQSEPRVRLVMAQHMIRDIQTLLSRMETLPYLQCRGGPQPQHSQPPPQPPAVTPEPVALSSQTSEPVESEAPPREPMEAEEVEERAPAQNPELTPGPAPAGPTPAPETNAPNHPSPAEYVEVLQELQRLESRLQPFLQRYYEVLGAAATTDYNNNHEGREEDQRLINLVGESLRLLGNTFVALSDLRCNLACTPPRHLHVVRPMSHYTTPMVLQQAAIPIQINVGTTVTMTGNGTRPPPTPNAEAPPPGPGQASSVAPSSTNVESSAEGAPPPGPAPPPATSHPRVIRISHQSVEPVVMMHMNIQDSGTQPGGVPSAPTGPLGPPGHGQTLGQQVPGFPTAPTRVVIARPTPPQARPSHPGGPPVSGTLQGAGLGTNASLAQMVSGLVGQLLMQPVLVAQGTPGMAPPPAPATASASAGTTNTATTAGPAPGGPAQPPPTPQPSMADLQFSQLLGNLLGPAGPGAGGSGVASPTITVAMPGVPAFLQGMTDFLQATQTAPPPPPPPPPPPPAPEQQTMPPPGSPSGGAGSPGGLGLESLSPEFFTSVVQGVLSSLLGSLGARAGSSESIAAFIQRLSGSSNIFEPGADGALGFFGALLSLLCQNFSMVDVVMLLHGHFQPLQRLQPQLRSFFHQHYLGGQEPTPSNIRMATHTLITGLEEYVRESFSLVQVQPGVDIIRTNLEFLQEQFNSIAAHVLHCTDSGFGARLLELCNQGLFECLALNLHCLGGQQMELAAVINGRIRRMSRGVNPSLVSWLTTMMGLRLQVVLEHMPVGPDAILRYVRRVGDPPQPLPEEPMEVQGAERASPEPQRENASPAPGTTAEEAMSRGPPPAPEGGSRDEQDGASAETEPWAAAVPPEWVPIIQQDIQSQRKVKPQPPLSDAYLSGMPAKRRKTMQGEGPQLLLSEAVSRAAKAAGARPLTSPESLSRDLEAPEVQESYRQQLRSDIQKRLQEDPNYSPQRFPNAQRAFADDP

SEQ ID NO: 40 - protein, 288 aa, Artificial Sequence
  Description of Artificial Sequence Synthetic polypeptide
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 41 - DNA, 690 bp, Artificial Sequence
  Description of Artificial Sequence Synthetic polynucleotide
gaattcaccggtgccgccaccatgaagtgggtgaccttcatcagcctgctgttcctgttcagcagcgcctacagccccggctggttcctggacagccccgaccgcccctggaacccccccaccttcagccccgccctgctggtggtgaccgagggcgacaacgccaccttcacctgcagcttcagcaacaccagcgagagcttcgtgctgaactggtaccgcatgagccccagcaaccagaccgacaagctggccgccttccccgaggaccgcagccagcccggccaggactgccgcttccgcgtgacccagctgcccaacggccgcgacttccacatgagcgtggtgcgcgcccgccgcaacgacagcggcacctacctgtgcggcgccatcagcctggcccccaaggcccagatcaaggagagcctgcgcgccgagctgcgcgtgaccgagcgccgcgccgaggtgcccaccgcccaccccagccccagcccccgccccgccggccagttccagaccctggtgaccggtggcgagatcgccgccctggagcaggagatcgccgccctggagaaggagaacgccgccctggagtgggagatcgccgccctggagcagggcggcggatccgacgacgacgacaagggcagcggctggagccacccccagttcgagaagtgatgagctagc

SEQ ID NO: 42 - protein, 220 aa, Artificial Sequence
  Description of Artificial Sequence Synthetic polypeptide
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 43 - protein, 223 aa, Artificial Sequence
  Description of Artificial Sequence Synthetic polypeptide
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 44 - protein, 199 aa, Artificial Sequence
  Description of Artificial Sequence Synthetic polypeptide
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 45 - protein, 306 aa, Artificial Sequence
  Description of Artificial Sequence Synthetic polypeptide
MKTVPAMLGTPRLFREFFILHLGLWSILCEKATKRNDEECPVQLTITRNSKQSARTGELFKIQCPVKYCVHRPNVTWCKHNGTICVPLEVSPQLYTSWEENQSVPVFVLHFKPIHLSDNGSYSCSTNFNSQVINSHSVTIHVRERTQNSSEHPLITVSDIPDATNASGPSTMEERPGRTWLLYTLLPLGALLLLLACVCLLCFLKRIQGKEKKPSDLAGRDTNLVDIPASSRTNHQALPSGTGIYDNDPWSSMQDESELTISLQSERNNQGIVYASLNHCVIGRNPRQENNMQEAPTEYASICVRS

SEQ ID NO: 46 - protein, 348 aa, Artificial Sequence [sequence omitted]

SEQ ID NO: 47 - protein, 348 aa, Artificial Sequence
  Description of Artificial Sequence Synthetic polypeptide
MSLMVVSMACVGFFLLQGAWPHEGVHRKPSLLAHPGRLVKSEETVILQCWSDVRFEHFLLHREGKFKDTLHLIGEHHDGVSKANFSIGPMMQDLAGTYRCYGSVTHSPYQLSAPSDPLDIVITGLYEKPSLSAQPGPTVLAGESVTLSCSSRSSYDMYHLSREGEAHECRFSAGPKVNGTFQADFPLGPATHGGTYRCFGSFRDSPYEWSNSSDPLLVSVIGNPSNSWPSPTEPSSKTGNPRHLHILIGTSVVIILFILLFFLLHRWCSNKKNAAVMDQESAGNRTANSEDSDEQDPQEVTYTQLNHCVFTQRKITRPSQRPKTPPTDIIVYAELPNAESRSKVVSCP

SEQ ID NO: 48 - protein, 341 aa, Artificial Sequence
  Description of Artificial Sequence Synthetic polypeptide
MSLMVVSMVCVGFFLLQGAWPHEGVHRKPSLLAHPGPLVKSEETVILQCWSDVRFQHFLLHREGKFKDTLHLIGEHHDGVSKANFSIGPMMQDLAGTYRCYGSVTHSPYQLSAPSDPLDIVITGLYEKPSLSAQPGPTVLAGESVTLSCSSRSSYDMYHLSREGEAHERRFSAGPKVNGTFQADFPLGPATHGGTYRCFGSFRDSPYEWSNSSDPLLVSVTGNPSNSWPSPTEPSSETGNPRHLHVLIGTSVVIILFILLLFFLLHRWCCNKKNAVVMDQEPAGNRTVNREDSDEQDPQEVTYAQLNHCVFTQRKITRPSQRPKTPPTDIIVYTELPNAEP

SEQ ID NO: 49 - protein, 377 aa, Artificial Sequence
  Description of Artificial Sequence Synthetic polypeptide
MSMSPTVIILACLGFFLDQSVWAHVGGQDKPFCSAWPSAVVPQGGHVTLRCHYRRGFNIFTLYKKDGVPVPELYNRIFWNSFLISPVTPAHAGTYRCRGFHPHSPTEWSAPSNPLVIMVTGLYEKPSLTARPGPTVRAGENVTLSCSSQSSFDIYHLSREGEAHELRLPAVPSINGTFQADFPLGPATHGETYRCFGSFHGSPYEWSDPSDPLPVSVTGNPSSSWPSPTEPSFKTGIARHLHAVIRYSVAIILFTILPFFLLHRWCSKKKDAAVMNQEPAGHRTVNREDSDEQDPQEVTYAQLDHCIFTQRKITGPSQRSKRPSTDTSVCIELPNAEPRALSPAHEHHSQALMGSSRETTALSQTQLASSNVPAAGI

SEQ ID NO: 50 - protein, 375 aa, Artificial Sequence
  Description of Artificial Sequence Synthetic polypeptide
MSLMVISMACVGFFLLQGAWTHEGGQDKPLLSAWPSAVVPRGGHVTLLCRSRLGFTIFSLYKEDGVPVPELYNKIFWKSILMGPVTPAHAGTYRCRGSHPRSPIEWSAPSNPLVIVVTGLFGKPSLSAQPGPTVRTGENVTLSCSSRSSFDMYHLSREGRAHEPRLPAVPSVNGTFQADFPLGPATHGGTYTCFGSLHDSPYEWSDPSDPLLVSVTGNSSSSSSSPTEPSSKTGIRRHLHILIGTSVAIILFIILFFFLLHCCCSNKKNAAVMDQEPAGDRTVNREDSDDQDPQEVTYAQLDHCVFTQTKITSPSQRPKTPPTDTTMYMELPNAKPRSLSPAHKHHSQALRGSSRETTALSQNRVASSHVPAAGI

SEQ ID NO: 51 - protein, 375 aa, Artificial Sequence [sequence omitted]

SEQ ID NO: 52 - protein, 304 aa, Artificial Sequence [sequence omitted]

SEQ ID NO: 53 - protein, 304 aa, Artificial Sequence
  Description of Artificial Sequence Synthetic polypeptide
MSLMVVSMACVGFFLLQGAWPHEGVHRKPSLLAHPGPLVKSEETVILQCWSDVRFEHFLLHREGKYKDTLHLIGEHHDGVSKANFSIGPMMQDLAGTYRCYGSVTHSPYQLSAPSDPLDIVITGLYEKPSLSAQPGPTVLAGESVTLSCSSRSSYDMYHLSREGEAHERRFSAGPKVNGTFQADFPLGPATHGGTYRCFGSFRDSPYEWSNSSDPLLVSVTGNPSNSWPSPTEPSSKTGNPRHLHVLIGTSVVKIPFTILLFFLLHRWCSNKKNAAVMDQEPAGNRTVNSEDSDEQDHQEVSYA

SEQ ID NO: 54 - protein, 304 aa, Artificial Sequence
  Description of Artificial Sequence Synthetic polypeptide
MSLMVISMACVGFFWLQGAWPHEGFRRKPSLLAHPGRLVKSEETVILQCWSDVMFEHFLLHREGTFNDTLRLIGEHIDGVSKANFSIGRMRQDLAGTYRCYGSVPHSPYQFSAPSDPLDIVITGLYEKPSLSAQPGPTVLAGESVTLSCSSWSSYDMYHLSTEGEAHERRFSAGPKVNGTFQADFPLGPATQGGTYRCFGSFHDSPYEWSKSSDPLLVSVTGNPSNSWPSPTEPSSKTGNPRHLHVLIGTSVVKLPFTILLFFLLHRWCSDKKNASVMDQGPAGNRTVNREDSDEQDHQEVSYA

SEQ ID NO: 55 - protein, 304 aa, Artificial Sequence
  Description of Artificial Sequence Synthetic polypeptide
MSLMVIIMACVGFFLLQGAWPQEGVHRKPSFLALPGHLVKSEETVILQCWSDVMFEHFLLHREGKFNNTLHLIGEHHDGVSKANFSIGPMMPVLAGTYRCYGSVPHSPYQLSAPSDPLDMVIIGLYEKPSLSAQPGPTVQAGENVTLSCSSRSSYDMYHLSREGEAHERRLPAVRSINGTFQADFPLGPATHGGTYRCFGSFRDAPYEWSNSSDPLLVSVTGNPSNSWPSPTEPSSKTGNPRHLHVLIGTSVVKIPFTILLFFLLHRWCSDKKNAAVMDQEPAGNRTVNSEDSDEQDHQEVSYA

SEQ ID NO: 56 - protein, 304 aa, Artificial Sequence
  Description of Artificial Sequence Synthetic polypeptide
MLLMVISMACVAFFLLQGAWPHEGFRRKPSLLAHPGPLVKSEETVILQCWSDVMFEHFLLHREGTFNHTLRLIGEHIDGVSKGNFSIGRMTQDLAGTYRCYGSVTHSPYQLSAPSDPLDIVITGLYEKPSLPAQPGPTVLAGESVTLSCSSRSSYDMYHLSREGEAHERRLPAGPKVNRTFQADSPLDPATHGGAYRCFGSFRDSPYEWSKSSDPLLVSVTGNSSNSWPSPTEPSSETGNPRHLHVLIGTSVVKLPFTILLFFLLHRWCSNKKNASVMDQGPAGNRTVNREDSDEQDHQEVSYA

SEQ ID NO: 57 - protein, 455 aa, Artificial Sequence
  Description of Artificial Sequence Synthetic polypeptide
MSLTVVSMACVGFFLLQGAWPLMGGQDKPFLSARPSTVVPRGGHVALQCHYRRGFNNFMLYKEDRSHVPIFHGRIFQESFIMGPVTPAHAGTYRCRGSRPHSLTGWSAPSNPLVIMVTGNHRKPSLLAHPGPLLKSGETVILQCWSDVMFEHFFLHREGISEDPSRLVGQIHDGVSKANFSIGPLMPVLAGTYRCYGSVPHSPYQLSAPSDPLDIVITGLYEKPSLSAQPGPTVQAGENVTLSCSSWSSYDIYHLSREGEAHERRLRAVPKVNRTFQADFPLGPATHGGTYRCFGSFRALPCVWSNSSDPLLVSVTGNPSSSWPSPTEPSSKSGICRHLHVLIGTSVVIFLFILLLFFLLYRWCSNKKNAAVMDQEPAGDRTVNRQDSDEQDPQEVTYAQLDHCVFIQRKISRPSQRPKTPLTDTSVYTELPNAEPRSKVVSCPRAPQSGLEGVF

SEQ ID NO: 58 - protein, 410 aa, Artificial Sequence
  Description of Artificial Sequence Synthetic polypeptide
MSLMVVSMACVGFFLLEGPWPHVGGQDKPFLSAWPGTVVSEGQHVTLQCRSRLGFNEFSLSKEDGMPVPELYNRIFRNSFLMGPVTPAHAGTYRCCSSHPHSPTGWSAPSNPVVIMVTGVHRKPSLLAHPGPLVKSGETVILQCWSDVRFERFLLHREGITEDPLRLVGQLHDAGSQVNYSMGPMTPALAGTYRCFGSVTHLPYELSAPSDPLDIVVVGLYGKPSLSAQPGPTVQAGENVTLSCSSRSLFDIYHLSREAEAGELRLTAVLRVNGTFQANFPLGPVTHGGNYRCFGSFRALPHAWSDPSDPLPVSVTGNSRHLHVLIGTSVVIIPFAILLFFLLHRWCANKKNAVVMDQEPAGNRTVNREDSDEQDPQEVTYAQLNHCVFTQRKITRPSQRPKTPPTDTSV

SEQ ID NO: 59 - protein, 387 aa, Artificial Sequence
  Description of Artificial Sequence Synthetic polypeptide
MSLMVVSMACVGLFLVQRAGPHMGGQDKPFLSAWPSAVVPRGGHVTLRCHYRHRFNNFMLYKEDRIHVPIFHGRIFQEGFNMSPVTTAHAGNYTCRGSHPHSPTGWSAPSNPMVIMVTGNHRKPSLLAHPGPLVKSGERVILQCWSDIMFEHFFLHKEWISKDPSRLVGQIHDGVSKANFSIGSMMRALAGTYRCYGSVTHTPYQLSAPSDPLDIVVTGLYEKPSLSAQPGPKVQAGESVTLSCSSRSSYDMYHLSREGGAHERRLPAVRKVNRTFQADFPLGPATHGGTYRCFGSFRHSPYEWSDPSDPLLVSVTGNPSSSWPSPTEPSSKSGNLRHLHILIGTSVVKIPFTILLFFLLHRWCSNKKNAAVMDQEPAGNRSEQRGF

SEQ ID NO: 60 - protein, 525 aa, Artificial Sequence
  Description of Artificial Sequence Synthetic polypeptide
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 61 - protein, 255 aa, Artificial Sequence
  Description of Artificial Sequence Synthetic polypeptide
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 62 - protein, 277 aa, Artificial Sequence
  Description of Artificial Sequence Synthetic polypeptide
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 63 - protein, 260 aa, Artificial Sequence
  Description of Artificial Sequence Synthetic polypeptide
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 64 - protein, 277 aa, Artificial Sequence
  Description of Artificial Sequence Synthetic polypeptide
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 65 - protein, 301 aa, Artificial Sequence
  Description of Artificial Sequence Synthetic polypeptide
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 66 - protein, 359 aa, Artificial Sequence
  Description of Artificial Sequence Synthetic polypeptide
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIAKKYFFKKEVQQLSVSFSSLQIKALQNAVEKEVQAEDNIYIENSLYATD

SEQ ID NO: 67 - protein, 305 aa, Artificial Sequence
  Description of Artificial Sequence Synthetic polypeptide
MNQIQVFISGLILLLPGAVESHTAVQGLAGHPVTLPCIYSTHLGGIVPMCWGLGECRHSYCIRSLIWTNGYTVTHQRNSLYQLKGNISEGNVSLTIENTVVGDGGPYCCVVEIPGAFHFVDYMLEVKPEISTSPPTRPTATGRPTTISTRSTHVPTSTRVSTSTSPTPAHTETYKPEATTFYPDQTTAEVTETLPDTPADWHNTVTSSDDPWDDNTEVIPPQKPQKNLNKGFYVGISIAALLILMLLSTMVITRYVVMKRKSESLSFVAFPISKIGASPKKVVERTRCEDQVYIIEDTPYPEEES

SEQ ID NO: 68 - protein, 378 aa, Artificial Sequence
  Description of Artificial Sequence Synthetic polypeptide
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLRGKLMETYCSQKHTRLDYIGDSKNVLNDVQHGREDEDGLFTL

SEQ ID NO: 69 - protein, 450 aa, Artificial Sequence
  Description of Artificial Sequence Synthetic polypeptide
MGSLQPDAGNASWNGTEAPGGGARATPYSLQVTLTLVCLAGLLMLLTVFGNVLVIIAVFTSRALKAPQNLFLVSLASADILVATLVIPFSLANEVMGYWYFGKAWCEIYLALDVLFCTSSIVHLCAISLDRYWSITQAIEYNLKRTPRRIKAIIITVWVISAVISFPPLISIEKKGGGGGPQPAEPRCEINDQKWYVISSCIGSFFAPCLIMILVYVRIYQIAKRRTRVPPSRRGPDAVAAPPGGTERRPNGLGPERSAGPGGAEAEPLPTQLNGAPGEPAPAGPRDTDALDLEESSSSDHAERPPGPRRPERGPRGKGKARASQVKPGDSLPRRGPGATGIGTPAAGPGEERVGAAKASRWRGRQNREKRFTFVLAVVIGVFVVCWFPFFFTYTLTAVGCSVPRTLFKFFFWFGYCNSSLNPVIYTIFNHDFRRAFKKILCRGDRKRIV

SEQ ID NO: 70 - protein, 20 aa, Artificial Sequence
  Description of Artificial Sequence Synthetic peptide
  MOD_RES (2)..(3): Any amino acid
  MOD_RES (4)..(4): Ile or Leu
  MOD_RES (5)..(16): Any amino acid
  MISC_FEATURE (5)..(16): This region may encompass 6-12 residues
  MOD_RES (18)..(19): Any amino acid
  MOD_RES (20)..(20): Ile or Leu
YXXXXXXXXXXXXXXXYXXX

SEQ ID NO: 71 - protein, 6 aa, Artificial Sequence
  Description of Artificial Sequence Synthetic peptide
  MOD_RES (1)..(1): Ser, Ile, Val or Leu
  MOD_RES (2)..(2): Any amino acid
  MOD_RES (4)..(5): Any amino acid
  MOD_RES (6)..(6): Ile, Val or Leu
XXYXXX

SEQ ID NO: 72 - protein, 6 aa, Artificial Sequence
  Description of Artificial Sequence Synthetic peptide
  MOD_RES (2)..(2): Any amino acid
  MOD_RES (4)..(5): Any amino acid
  MOD_RES (6)..(6): Val or Ile
TXYXXX

SEQ ID NO: 73 - protein, 370 aa, Artificial Sequence
  Description of Artificial Sequence Synthetic polypeptide
MLGQVVTLILLLLLKVYQGKGCQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 74 - protein, 244 aa, Artificial Sequence
  Description of Artificial Sequence Synthetic polypeptide
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 75 - protein, 290 aa, Artificial Sequence
  Description of Artificial Sequence Synthetic polypeptide
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 76 - DNA, 897 bp, Artificial Sequence
  Description of Artificial Sequence Synthetic polynucleotide
gaattcaccggtgccgccaccatgaagtgggtgaccttcatcagcctgctgttcctgttcagcagcgcctacagcaccgtgaccgtgcccaaggacctgtacgtggtggagtacggcagcaacatgaccatcgagtgcaagttccccgtggagaagcagctggacctggccgccctgatcgtgtactgggagatggaggacaagaacatcatccagttcgtgcacggcgaggaggacctgaaggtgcagcacagcagctaccgccagcgcgcccgcctgctgaaggaccagctgagcctgggcaacgccgccctgcagatcaccgacgtgaagctgcaggacgccggcgtgtaccgctgcatgatcagctacggcggcgccgactacaagcgcatcaccgtgaaggtgaacgccccctacaacaagatcaaccagcgcatcctggtggtggaccccgtgaccagcgagcacgagctgacctgccaggccgagggctaccccaaggccgaggtgatctggaccagcagcgaccaccaggtgctgagcggcaagaccaccaccaccaacagcaagcgcgaggagaagctgttcaacgtgaccagcaccctgcgcatcaacaccaccaccaacgagatcttctactgcaccttccgccgcctggaccccgaggagaaccacaccgccgagctggtgatccccgagctgcccctggcccacccccccaacgagcgcaccggtggcgagatcgccgccctggagcaggagatcgccgccctggagaaggagaacgccgccctggagtgggagatcgccgccctggagcagggcggcggatccgacgacgacgacaagggcagcggctggagccacccccagttcgagaagtgatgagctagc

SEQ ID NO: 77 - protein, 273 aa, Artificial Sequence
  Description of Artificial Sequence Synthetic polypeptide
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 78 - protein, 288 aa, Artificial Sequence
  Description of Artificial Sequence Synthetic polypeptide
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 79 - protein, 329 aa, Artificial Sequence
  Description of Artificial Sequence Synthetic polypeptide
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 80 - protein, 302 aa, Artificial Sequence
  Description of Artificial Sequence Synthetic polypeptide
MRLGSPGLLFLLFSSLRADTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSILAVLCLLVVVAVAIGWVCRDRCLQHSYAGAWAVSPETELTGHV

SEQ ID NO: 81 - protein, 534 aa, Artificial Sequence
  Description of Artificial Sequence Synthetic polypeptide
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 82 - protein, 282 aa, Artificial Sequence
  Description of Artificial Sequence Synthetic polypeptide
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 83 - protein, 283 aa, Artificial Sequence
  Description of Artificial Sequence Synthetic polypeptide
MEPPGDWGPPPWRSTPKTDVLRLVLYLTFLGAPCYAPALPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWVWWFLSGSLVIVIVCSTVGLIICVKRRKPRGDVVKVIVSVQRKRQEAEGEATVIEALQAPPDVTTVAVEETIPSFTGRSPNH

SEQ ID NO: 84 - protein, 365 aa, Artificial Sequence [sequence omitted]

SEQ ID NO: 85 - protein, 362 aa, Artificial Sequence
  Description of Artificial Sequence Synthetic polypeptide
MLVMAPRTVLLLLSAALALTETWAGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 86 - protein, 366 aa, Artificial Sequence
  Description of Artificial Sequence Synthetic polypeptide
MRVMAPRALLLLLSGGLALTETWACSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKA

SEQ ID NO: 87 - protein, 261 aa, Artificial Sequence
  Description of Artificial Sequence Synthetic polypeptide
MGHEQNQGAALLQMLPLLWLLPHSWAVPEAPTPMWPDDLQNHTFLHTVYCQDGSPSVGLSEAYDEDQLFFFDFSQNTRVPRLPEFADWAQEQGDAPAILFDKEFCEWMIQQIGPKLDGKIPVSRGFPIAEVFTLKPLEFGKPNTLVCFVSNLFPPMLTVNWHDHSVPVEGFGPTFVSAVDGLSFQAFSYLNFTPEPSDIFSCIVTHEIDRYTAIAYWVPRNALPSDLLENVLCGVAFGLGVLGIIVGIVLIIYFRKPCSGD

SEQ ID NO: 88 - protein, 263 aa, Artificial Sequence
  Description of Artificial Sequence Synthetic polypeptide
MITFLPLLLGLSLGCTGAGGFVAHVESTCLLDDAGTPKDFTYCISFNKDLLTCWDPEENKMAPCEFGVLNSLANVLSQHLNQKDTLMQRLRNGLQNCATHTQPFWGSLTNRTRPPSVQVAKTTPFNTREPVMLACYVWGFYPAEVTITWRKNGKLVMPHSSAHKTAQPNGDWTYQTLSHLALTPSYGDTYTCVVEHIGAPEPILRDWTPGLSPMQTLKVSVSAVTLGLGLIIFSLGVISWRRAGHSSYTPLPGSNYSEGWHIS

SEQ ID NO: 89 - protein, 250 aa, Artificial Sequence
  Description of Artificial Sequence Synthetic polypeptide
MALRAGLVLGFHTLMTLLSPQEAGATKADHMGSYGPAFYQSYGASGQFTHEFDEEQLFSVDLKKSEAVWRLPEFGDFARFDPQGGLAGIAAIKAHLDILVERSNRSRAINVPPRVTVLPKSRVELGQPNILICIVDNIFPPVINITWLRNGQTVTEGVAQTSFYSQPDHLFRKFHYLPFVPSAEDVYDCQVEHWGLDAPLLRHWELQVPIPPPDAMETLVCALGLAIGLVGFLVGTVLIIMGTYVSSVPR

SEQ ID NO: 90 - protein, 273 aa, Artificial Sequence
  Description of Artificial Sequence Synthetic polypeptide
MGSGWVPWVVALLVNLTRLDSSMTQGTDSPEDFVIQAKADCYFTNGTEKVQFVVRFIFNLEEYVRFDSDVGMFVALTKLGQPDAEQWNSRLDLLERSRQAVDGVCRHNYRLGAPFTVGRKVQPEVTVYPERTPLLHQHNLLHCSVTGFYPGDIKIKWFLNGQEERAGVMSTGPIRNGDWTFQTVVMLEMTPELGHVYTCLVDHSSLLSPVSVEWRAQSEYSWRKMLSGIAAFLLGLIFLLVGIVIQLRAQKGYVRTQMSGNEVSRAVLLPQSC

SEQ ID NO: 91 - protein, 260 aa, Artificial Sequence
  Description of Artificial Sequence Synthetic polypeptide
MRPEDRMFHIRAVILRALSLAFLLSLRGAGAIKADHVSTYAAFVQTHRPTGEFMFEFDEDEMFYVDLDKKETVWHLEEFGQAFSFEAQGGLANIAILNNNLNTLIQRSNHTQATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHWEAQEPIQMPETTETVLCALGLVLGLVGIIVGTVLIIKSLRSGHDPRAQGTL

SEQ ID NO: 92 - protein, 258 aa, Artificial Sequence
  Description of Artificial Sequence Synthetic polypeptide
MMVLQVSAAPRTVALTALLMVLLTSVVQGRATPENYLFQGRQECYAFNGTQRFLERYIYNREEFARFDSDVGEFRAVTELGRPAAEYWNSQKDILEEKRAVPDRMCRHNYELGGPMTLQRRVQPRVNVSPSKKGPLQHHNLLVCHVTDFYPGSIQVRWFLNGQEETAGVVSTNLIRNGDWTFQILVMLEMTPQQGDVYTCQVEHTSLDSPVTVEWKAQSDSARSKTLTGAGGFVLGLIICGVGIFMHRRSKKVQRGSA

SEQ ID NO: 93 - protein, 254 aa, Artificial Sequence
  Description of Artificial Sequence Synthetic polypeptide
MILNKALMLGALALTTVMSPCGGEDIVADHVASYGVNLYQSYGPSGQYTHEFDGDEQFYVDLGRKETVWCLPVLRQFRFDPQFALTNIAVLKHNLNSLIKRSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTETVVCALGLSVGLVGIVVGTVFIIRGLRSVGASRHQGPL

SEQ ID NO: 94 - protein, 261 aa, Artificial Sequence
  Description of Artificial Sequence Synthetic polypeptide
MSWKKALRIPGGLRAATVTLMLAMLSTPVAEGRDSPEDFVYQFKAMCYFTNGTERVRYVTRYIYNREEYARFDSDVEVYRAVTPLGPPDAEYWNSQKEVLERTRAELDTVCRHNYQLELRTTLQRRVEPTVTISPSRTEALNHHNLLVCSVTDFYPAQIKVRWFRNDQEETTGVVSTPLIRNGDWTFQILVMLEMTPQHGDVYTCHVEHPSLQNPITVEWRAQSESAQSKMLSGIGGFVLGLIFLGLGLIIHHRSQKGLLH

SEQ ID NO: 95 - protein, 254 aa, Artificial Sequence
  Description of Artificial Sequence Synthetic polypeptide
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGVRKSNAAERRGPL

SEQ ID NO: 96 - protein, 266 aa, Artificial Sequence [sequence omitted]

SEQ ID NO: 97 - protein, 254 aa, Artificial Sequence; identical to SEQ ID NO: 14
  Description of Artificial Sequence Synthetic polypeptide

SEQ ID NO: 98 - protein, 183 aa, Artificial Sequence; identical to SEQ ID NO: 6
  Description of Artificial Sequence Synthetic polypeptide

SEQ ID NO: 99 - protein, 193 aa, Artificial Sequence; identical to SEQ ID NO: 12
  Description of Artificial Sequence Synthetic polypeptide

SEQ ID NO: 100 - protein, 355 aa, Artificial Sequence
  Description of Artificial Sequence Synthetic polypeptide
MAFSGSQAPYLSPAVPFSGTIQGGLQDGLQITVNGTVLSSSGTRFAVNFQTGFSGNDIAFHFNPRFEDGGYVVCNTRQNGSWGPEERKTHMPFQKGMPFDLCFLVQSSDFKVMVNGILFVQYFHRVPFHRVDTISVNGSVQLSYISFQNPRTVPVQPAFSTVPFSQPVCFPPRPRGRRQKPPGVWPANPAPITQTVIHTVQSAPGQMFSTPAIPPMMYPHPAYPMPFITTILGGLYPSKSILLSGTVLPSAQRFHINLCSGNHIAFHLNPRFDENAVVRNTQIDNSWGSEERSLPRKMPFVRGQSFSVWILCEAHCLKVAVDGQHLFEYYHRLRNLPTINRLEVGGDIQLTHVQT

SEQ ID NO: 101 - protein, 271 aa, Artificial Sequence
  Description of Artificial Sequence Synthetic polypeptide
MAKVPDMFEDLKNCYSENEEDSSSIDHLSLNQKSFYHVSYGPLHEGCMDQSVSLSISETSKTSKLTFKESMVVVATNGKVLKKRRLSLSQSITDDDLEAIANDSEEEIIKPRSAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 102 - protein, 269 aa, Artificial Sequence
  Description of Artificial Sequence Synthetic polypeptide
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 103 - protein, 177 aa, Artificial Sequence
  Description of Artificial Sequence Synthetic polypeptide
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 104 - protein, 270 aa, Artificial Sequence
  Description of Artificial Sequence Synthetic polypeptide
MKPKMKYSTNKISTAKWKNTASKALCFKLGKSQQKAKEVCPMYFMKLRSGLMIKKEACYFRRETTKRPSLKTGRKHKRHLVLAACQQQSTVECFAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 105 - protein, 189 aa, Artificial Sequence
  Description of Artificial Sequence Synthetic polypeptide
MAAEPVEDNCINFVAMKFIDNTLYFIENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 106 - protein, 155 aa, Artificial Sequence
  Description of Artificial Sequence Synthetic polypeptide
MVLSGALCFRMKDSALKVLYLHNNQLLAGGLHAGKVIKGEEISVVPNRWLDASLSPVILGVQGGSQCLSCGVGQEPTLTLEPVNIMELYLGAKESKSFTFYRRDMGLTSSFESAAYPGWFLCTVPEADQPVRLTQLPENGGWNAPITDFYFQQCD

SEQ ID NO: 107 - protein, 158 aa, Artificial Sequence
  Description of Artificial Sequence Synthetic polypeptide
MEKALKIDTPQQGSIQDINHRVWVLQDQTLIAVPRKDRMSPVTIALISCRHVETLEKDRGNPIYLGLNGLNLCLMCAKVGDQPTLQLKEKDIMDLYNQPEPVKSFLFYHSQSGRNSTFESVAFPGWFIAVSSEGGCPLILTQELGKANTTDFGLTMLF

SEQ ID NO: 108 - protein, 164 aa, Artificial Sequence
  Description of Artificial Sequence Synthetic polypeptide
MNPQREAAPKSYAIRDSRQMVWVLSGNSLIAAPLSRSIKPVTLHLIACRDTEFSDKEKGNMVYLGIKGKDLCLFCAEIQGKPTLQLKLQGSQDNIGKDTCWKLVGIHTCINLDVRESCFMGTLDQWGIGVGRKKWKSSFQHHHLRKKDKDFSSMRTNIGMPGRM

SEQ ID NO: 109 - protein, 169 aa, Artificial Sequence
  Description of Artificial Sequence Synthetic polypeptide
MRGTPGDADGGGRAVYQSMCKPITGTINDLNQQVWTLQGQNLVAVPRSDSVTPVTVAVITCKYPEALEQGRGDPIYLGIQNPEMCLYCEKVGEQPTLQLKEQKIMDLYGQPEPVKPFLFYRAKTGRTSTLESVAFPDWFIASSKRDQPIILTSELGKSYNTAFELNIND

SEQ ID NO: 110 - protein, 218 aa, Artificial Sequence
  Description of Artificial Sequence Synthetic polypeptide
MSFVGENSGVKMGSEDWEKDEPQCCLEDPAGSPLEPGPSLPTMNFVHTSPKVKNLNPKKFSIHDQDHKVLVLDSGNLIAVPDKNYIRPEIFFALASSLSSASAEKGSPILLGVSKGEFCLYCDKDKGQSHPSLQLKKEKLMKLAAQKESARRPFIFYRAQVGSWNMLESAAHPGWFICTSCNCNEPVGVTDKFENRKHIEFSFQPVCKAEMSPSEVSD

SEQ ID NO: 111 - protein, 152 aa, Artificial Sequence
  Description of Artificial Sequence Synthetic polypeptide
MCSLPMARYYIIKYADQKALYTRDGQLLVGDPVADNCCAEKICILPNRGLARTKVPIFLGIQGGSRCLACVETEEGPSLQLEDVNIEELYKGGEEATRFTFFQSSSGSAFRLEAAAWPGWFLCGPAEPQQPVQLTKESEPSARTKFYFEQSW

SEQ ID NO: 112 - protein, 153 aa, Artificial Sequence
  Description of Artificial Sequence Synthetic polypeptide
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 113 - protein, 152 aa, Artificial Sequence
  Description of Artificial Sequence Synthetic polypeptide
MSRLPVLLLLQLLVRPGLQAPMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 114 - protein, 153 aa, Artificial Sequence [sequence omitted]

SEQ ID NO: 115 - protein, 134 aa, Artificial Sequence
  Description of Artificial Sequence Synthetic polypeptide
MRMLLHLSLLALGAAYVYAIPTEIPTSALVKETLALLSTHRTLLIANETLRIPVPVHKNHQLCTEEIFQGIGTLESQTVQGGTVERLFKNLSLIKKYIDGQKKKCGEERRRVNQFLDYLQEFLGVMNTEWIIES

SEQ ID NO: 116 - protein, 212 aa, Artificial Sequence
  Description of Artificial Sequence Synthetic polypeptide
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 117 - protein, 177 aa, Artificial Sequence
  Description of Artificial Sequence Synthetic polypeptide
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 118 - protein, 99 aa, Artificial Sequence
  Description of Artificial Sequence Synthetic polypeptide
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 119 - protein, 144 aa, Artificial Sequence
  Description of Artificial Sequence Synthetic polypeptide
MLLAMVLTSALLLCSVAGQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 120 - protein, 178 aa, Artificial Sequence
  Description of Artificial Sequence Synthetic polypeptide
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 121 - protein, 199 aa, Artificial Sequence
  Description of Artificial Sequence Synthetic polypeptide
MNCVCRLVLVVLSLWPDTAVAPGPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRL

SEQ ID NO: 122 - protein, 219 aa, Artificial Sequence
  Description of Artificial Sequence Synthetic polypeptide
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 123 - protein, 328 aa, Artificial Sequence
  Description of Artificial Sequence Synthetic polypeptide
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 124 - protein, 146 aa, Artificial Sequence [sequence omitted]

SEQ ID NO: 125 - protein, 546 aa, Artificial Sequence
  Description of Artificial Sequence Synthetic polypeptide
MKNQDKKNGAAKQSNPKSSPGQPEAGPEGAQERPSQAAPAVEAEGPGSSQAPRKPEGAQARTAQSGALRDVSEELSRQLEDILSTYCVDNNQGGPGEDGAQGEPAEPEDAEKSRTYVARNGEPEPTPVVNGEKEPSKGDPNTEEIRQSDEVGDRDHRRPQEKKKAKGLGKEITLLMQTLNTLSTPEEKLAALCKKYAELLEEHRNSQKQMKLLQKKQSQLVQEKDHLRGEHSKAVLARSKLESLCRELQRHNRSLKEEGVQRAREEEEKRKEVTSHFQVTLNDIQLQMEQHNERNSKLRQENMELAERLKKLIEQYELREEHIDKVFKHKDLQQQLVDAKLQQAQEMLKEAEERHQREKDFLLKEAVESQRMCELMKQQETHLKQQLALYTEKFEEFQNTLSKSSEVFTTFKQEMEKMTKKIKKLEKETTMYRSRWESSNKALLEMAEEKTVRDKELEGLQVKIQRLEKLCRALQTERNDLNKRVQDLSAGGQGSLTDSGPERRPEGPGAQAPSSPRVTEAPCYPGAPSTEASGQTGPQEPTSARA

SEQ ID NO: 126 - protein, 162 aa, Artificial Sequence
  Description of Artificial Sequence Synthetic polypeptide
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 127 - protein, 1332 aa, Artificial Sequence
  Description of Artificial Sequence Synthetic polypeptide
MESHSRAGKSRKSAKFRSISRSLMLCNAKTSDDGSSPDEKYPDPFEISLAQGKEGIFHSSVQLADTSEAGPSSVPDLALASEAAQLQAAGNDRGKTCRRIFFMKESSTASSREKPGKLEAQSSNFLFPKACHQRARSNSTSVNPYCTREIDFPMTKKSAAPTDRQPYSLCSNRKSLSQQLDCPAGKAAGTSRPTRSLSTAQLVQPSGGLQASVISNIVLMKGQAKGLGFSIVGGKDSIYGPIGIYVKTIFAGGAAAADGRLQEGDEILELNGESMAGLTHQDALQKFKQAKKGLLTLTVRTRLTAPPSLCSHLSPPLCRSLSSSTCITKDSSSFALESPSAPISTAKPNYRIMVEVSLQKEAGVGLGIGLCSVPYFQCISGIFVHTLSPGSVAHLDGRLRCGDEIVEISDSPVHCLTLNEVYTILSHCDPGPVPIIVSRHPDPQVSEQQLKEAVAQAVENTKFGKERHQWSLEGVKRLESSWHGRPTLEKEREKNSAPPHRRAQKVMIRSSSDSSYMSGSPGGSPGSGSAEKPSSDVDISTHSPSLPLAREPVVLSIASSRLPQESPPLPESRDSHPPLRLKKSFEILVRKPMSSKPKPPPRKYFKSDSDPQKSLEERENSSCSSGHTPPTCGQEARELLPLLLPQEDTAGRSPSASAGCPGPGIGPQTKSSTEGEPGWRRASPVTQTSPIKHPLLKRQARMDYSFDTTAEDPWVRISDCIKNLFSPIMSENHGHMPLQPNASLNEEEGTQGHPDGTPPKLDTANGTPKVYKSADSSTVKKGPPVAPKPAWFRQSLKGLRNRASDPRGLPDPALSTQPAPASREHLGSHIRASSSSSSIRQRISSFETFGSSQLPDKGAQRLSLQPSSGEAAKPLGKHEEGRFSGLLGRGAAPTLVPQQPEQVLSSGSPAASEARDPGVSESPPPGRQPNQKTLPPGPDPLLRLLSTQAEESQGPVLKMPSQRARSFPLTRSQSCETKLLDEKTSKLYSISSQVSSAVMKSLLCLPSSISCAQTPCIPKEGASPTSSSNEDSAANGSAETSALDTGFSLNLSELREYTEGLTEAKEDDDGDHSSLQSGQSVISLLSSEELKKLIEEVKVLDEATLKQLDGIHVTILHKEEGAGLGFSLAGGADLENKVITVHRVFPNGLASQEGTIQKGNEVLSINGKSLKGTTHHDALAILRQAREPRQAVIVTRKLTPEAMPDLNSSTDSAASASAASDVSVESTAEATVCTVTLEKMSAGLGFSLEGGKGSLHGDKPLTINRIFKGAASEQSETVQPGDEILQLGGTAMQGLTRFEAWNIIKALPDGPVTIVIRRKSLQSKETTAAGDS

SEQ ID NO: 128 - protein, 155 aa, Artificial Sequence
  Description of Artificial Sequence Synthetic polypeptide
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 129 - protein, 180 aa, Artificial Sequence
  Description of Artificial Sequence Synthetic polypeptide
MDWPHNLLFLLTISIFLGLGQPRSPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 130 - protein, 197 aa, Artificial Sequence
  Description of Artificial Sequence Synthetic polypeptide
MTLLPGLLFLTWLHTCLAHHDPSLRGHPHSHGTPHCYSAEELPLGQAPPHLLARGAKWGQALPVALVSSLEAASHRGRHERPSATTQCPVLRPEEVLEADTHQRSISPWRYRVDTDEDRYPQKLAFAECLCRGCIDARTGRETAALNSVRLLQSLLVLRRRPCSRDGSGLPTPGAFAFHTEFIHVPVGCTCVLPRSV

SEQ ID NO: 131 - protein, 202 aa, Artificial Sequence
  Description of Artificial Sequence Synthetic polypeptide
MLVAGFLLALPPSWAAGAPRAGRRPARPRGCADRPEELLEQLYGRLAAGVLSAFHHTLQLGPREQARNASCPAGGRPADRRFRPPTNLRSVSPWAYRISYDPARYPRYLPEAYCLCRGCLTGLFGEEDVRFRSAPVYMPTVVLRRTPACAGGRSVYTEAYVTIPVGCTCVPEPEKDADSINSSIDKQGAKLLLGPNDAPAGP

SEQ ID NO: 132 - protein, 163 aa, Artificial Sequence
  Description of Artificial Sequence Synthetic polypeptide
MTVKTLHGPAMVKYLLLSILGLAFLSEAAARKIPKVGHTFFQKPESCPPVPGGSMKLDIGIINENQRVSMSRNIESRSTSPWNYTVTWDPNRYPSEVVQAQCRNLGCINAQGKEDISMNSVPIQQETLVVRRKHQGCSVSFQLEKVLVTVGCTCVTPVIHHVQ

SEQ ID NO: 133 - protein, 177 aa, Artificial Sequence
  Description of Artificial Sequence Synthetic polypeptide
MKLQCVSLWLLGTILILCSVDNHGLRRCLISTDMHHIEESFQEIKRAIQAKDTFPNVTILSTLETLQIIKPLDVCCVTKNLLAFYVDRVFKDHQEPNPKILRKISSIANSFLYMQKTLRQCQEQRQCHCRQEATNATRVIHDNYDQLEVHAAAIKSLGELDVFLAWINKNHEVMFSA

SEQ ID NO: 134 - protein, 176 aa, Artificial Sequence
  Description of Artificial Sequence Synthetic polypeptide
MKASSLAFSLLSAAFYLLWTPSTGLKTLNLGSCVIATNLQEIRNGFSEIRGSVQAKDGNIDIRILRRTESLQDTKPANRCCLLRHLLRLYLDRVFKNYQTPDHYTLRKISSLANSFLTIKKDLRLCHAHMTCHCGEEAMKKYSQILSHFEKLEPQAAVVKALGELDILLQWMEETE

SEQ ID NO: 135 - protein, 155 aa, Artificial Sequence
  Description of Artificial Sequence Synthetic polypeptide
MERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 136 - protein, 179 aa, Artificial Sequence
  Description of Artificial Sequence Synthetic polypeptide
MAALQKSVSSFLMGTLATSCLLLLALLVQGGAAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 137 - protein, 189 aa, Artificial Sequence
  Description of Artificial Sequence Synthetic polypeptide
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 138 - protein, 206 aa, Artificial Sequence
  Description of Artificial Sequence Synthetic polypeptide
MNFQQRLQSLWTLARPFCPPLLATASQMQMVVLPCLGFTLLLWSQVSGAQGQEFHFGPCQVKGVVPQKLWEAFWAVKDTMQAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 139 - protein, 177 aa, Artificial Sequence
  Description of Artificial Sequence Synthetic polypeptide
MRERPRLGEDSSLISLFLQVVAFLAMVMGTHTYSHWPSCCPSKGQDTSEELLRWSTVPVPPLEPARPNRHPESCRASEDGPLNSRAISPWRYELDRDLNRLPQDLYHARCLCPHCVSLQTGSHMDPRGNSELLYHNQTVFYRRPCHGEKGTHKGYCLERRLYRVSLACVCVRPRVMG

SEQ ID NO: 140 - protein, 171 aa, Artificial Sequence
  Description of Artificial Sequence Synthetic polypeptide
MLVNFILRCGLLLVTLSLAIAKHKQSSFTKSCYPRGTLSQAVDALYIKAAWLKATIPEDRIKNIRLLKKKTKKQFMKNCQFQEQLLSFFMEDVFGQLQLQGCKKIRFVEDFHSLRQKLSHCISCASSAREMKSITRMKRIFYRIGNKGIYKAISELDILLSWIKKLLESSQ

SEQ ID NO: 141 - protein, 243 aa, Artificial Sequence
  Description of Artificial Sequence Synthetic polypeptide
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQP

SEQ ID NO: 142 - protein, 229 aa, Artificial Sequence
  Description of Artificial Sequence Synthetic polypeptide
MTPQLLLALVLWASCPPCSGRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 143 - protein, 200 aa, Artificial Sequence
  Description of Artificial Sequence Synthetic polypeptide
MKLDMTGDCTPVLVLMAAVLTVTGAVPVARLHGALPDARGCHIAQFKSLSPQELQAFKRAKDALEESLLLKDCRCHSRLFPRTWDLRQLQVRERPMALEAELALTLKVLEATADTDPALVDVLDQPLHTLHHILSQFRACIQPQPTAGPRTRGRLHHWLYRLQEAPKKESPGCLEASVTFNLFRLLTRDLNCVASGDLCV

SEQ ID NO: 144 - protein, 196 aa, Artificial Sequence
  Description of Artificial Sequence Synthetic polypeptide
MTGDCMPVLVLMAAVLTVTGAVPVARLRGALPDARGCHIAQFKSLSPQELQAFKRAKDALEESLLLKDCKCRSRLFPRTWDLRQLQVRERPVALEAELALTLKVLEATADTDPALGDVLDQPLHTLHHILSQLRACIQPQPTAGPRTRGRLHHWLHRLQEAPKKESPGCLEASVTFNLFRLLTRDLNCVASGDLCV

SEQ ID NO: 145 - protein, 200 aa, Artificial Sequence
  Description of Artificial Sequence Synthetic polypeptide
MAAAWTVVLVTLVLGLAVAGPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLCLRTSTHPEST

SEQ ID NO: 146 - protein, 164 aa, Artificial Sequence
  Description of Artificial Sequence Synthetic polypeptide
MASHSGPSTSVLFLFCCLGGWLASHTLPVRLLRPSDDVQKIVEELQSLSKMLLKDVEEEKGVLVSQNYTLPCLSPDAQPPNNIHSPAIRAYLKTIRQLDNKSVIDEIIEHLDKLIFQDAPETNISVPTDTHECKRFILTISQQFSECMDLALKSLTSGAQQATT

SEQ ID NO: 147 - protein, 234 aa, Artificial Sequence
  Description of Artificial Sequence Synthetic polypeptide
MCFPKVLSDDMKKLKARMVMLLPTSAQGLGAWVSACDTEDTVGHLGPWRDKDPALWCQLCLSSQHQAIERFYDKMQNAESGRGQVMSSLAELEDDFKEGYLETVAAYYEEQHPELTPLLEKERDGLRCRGNRSPVPDVEDPATEEPGESFCDKVMRWFQAMLQRLQTWWHGVLAWVKEKVVALVHAVQALWKQFQSFCCSLSELFMSSFQSYGAPRGDKEELTPQKCSEPQSSK

SEQ ID NO: 148 - protein, 390 aa, Artificial Sequence
  Description of Artificial Sequence Synthetic polypeptide
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 149 - protein, 414 aa, Artificial Sequence
  Description of Artificial Sequence Synthetic polypeptide
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 150 - protein, 412 aa, Artificial Sequence
  Description of Artificial Sequence Synthetic polypeptide
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 151 - protein, 31 aa, Artificial Sequence
  Description of Artificial Sequence Synthetic polypeptide
GEIAALEQEIAALEKENAALEWEIAALEQGG

SEQ ID NO: 152 - protein, 31 aa, Artificial Sequence
  Description of Artificial Sequence Synthetic polypeptide
GKIAALKQKIAALKYKNAALKKKIAALKQGG

SEQ ID NO: 153 - protein, 38 aa, Artificial Sequence
  Description of Artificial Sequence Synthetic polypeptide
CGGREGVLKKLRAVENELHYNKSLLEEVKDELQKMRQL

SEQ ID NO: 154 - DNA, 7599 bp, Artificial Sequence
  Description of Artificial Sequence Synthetic polynucleotide
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagacatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatccggtaccgaggagatctgccgccgcgatcgccggcgcgccagatctcaagcttaactagctagcggaccgacgcgtacgcggccgctcgagcagaaactcatctcagaagaggatctggcagcaaatgatatcctggattacaaggatgacgacgataaggtttaaacctaggcgtagcggccgcaaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatgaccgagtacaagcccacggtgcgcctcgccacccgcgacgacgtcccccgggcagtacgcaccctcgccgccgcgttcgccgactaccccgccacgcgccacaccgtcgatccagaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgatgtacaagtaggattcgtcgagggacctaataacttcgtatagcatacattatacgaagttatacatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccatactcttcctttttcaatattattgaagcatttatcagggttattgtctcatgagcggatacatatttgaatgtatttagaaaaataaacaaataggggtcccgcgcacatttccccgaaaagtgccacctgac

SEQ ID NO: 155 - DNA, 8711 bp, Artificial Sequence
  Description of Artificial Sequence Synthetic polynucleotide
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagacatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcaccggtgccgccaccatggcctgcctgggcttccagcgccacaaggcccagctgaacctggccacccgcacctggccctgcaccctgctgttcttcctgctgttcatccccgtgttctgcaaggccatgcacgtggcccagcccgccgtggtgctggccagcagccgcggcatcgccagcttcgtgtgcgagtacgccagccccggcaaggccaccgaggtgcgcgtgaccgtgctgcgccaggccgacagccaggtgaccgaggtgtgcgccgccacctacatgatgggcaacgagctgaccttcctggacgacagcatctgcaccggcaccagcagcggcaaccaggtgaacctgaccatccagggcctgcgcgccatggacaccggcctgtacatctgcaaggtggagctgatgtacccccccccctactacctgggcatcggcaacggcacccagatctacgtgatcaccggtgagcccaagagctgcgacaagacccacacctgccccccctgccccgcccccgagctgctgggcggccccagcgtgttcctgttcccccccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctgcaccaggactggctgaacggcaaggagtacaagtgcaaggtgagcaacaaggccctgcccgcccccatcgagaagaccatcagcaaggccaagggccagccccgcgagccccaggtgtacaccctgccccccagccgcgacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccgctggcagcagggcaacgtgttcagctgcagcgtgatgcacgaggccctgcacaaccactacacccagaagagcctgagcctgagccccggcaagggatcctgagctagcggaccgacgcgtacgcggccgctcgagcagaaactcatctcagaagaggatctggcagcaaatgatatcctggattacaaggatgacgacgataaggtttaaacctaggcgtagcggccgcaaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatgaccgagtacaagcccacggtgcgcctcgccacccgcgacgacgtcccccgggcagtacgcaccctcgccgccgcgttcgccgactaccccgccacgcgccacaccgtcgatccagaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgatgtacaagtaggattcgtcgagggacctaataacttcgtatagcatacattatacgaagttatacatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccatactcttcctttttcaatattattgaagcatttatcagggttattgtctcatgagcggatacatatttgaatgtatttagaaaaataaacaaataggggtcccgcgcacatttccccgaaaagtgccacctgac

SEQ ID NO: 156 - DNA, 8297 bp, Artificial Sequence
  Description of Artificial Sequence Synthetic polynucleotide
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagacatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcaccggtgccgccaccatgaagtgggtgaccttcatcagcctgctgttcctgttcagcagcgcctacagctggagccacccccagttcgagaagggcagcggcgacgacgacgacaagggcagcggcggcaagatcgccgccctgaagcagaagatcgccgccctgaagtacaagaacgccgccctgaagaagaagatcgccgccctgaagcagggcggcggatcccagctgttccacctgcagaaggagctggccgagctgcgcgagagcaccagccagatgcacaccgccagcagcctggagaagcagatcggccaccccagccccccccccgagaagaaggagctgcgcaaggtggcccacctgaccggcaagagcaacagccgcagcatgcccctggagtgggaggacacctacggcatcgtgctgctgagcggcgtgaagtacaagaagggcggcctggtgatcaacgagaccggcctgtacttcgtgtacagcaaggtgtacttccgcggccagagctgcaacaacctgcccctgagccacaaggtgtacatgcgcaacagcaagtacccccaggacctggtgatgatggagggcaagatgatgagctactgcaccaccggccagatgtgggcccgcagcagctacctgggcgccgtgttcaacctgaccagcgccgaccacctgtacgtgaacgtgagcgagctgagcctggtgaacttcgaggagagccagaccttcttcggcctgtacaagctgtgatgagctagcggaccgacgcgtacgcggccgctcgagcagaaactcatctcagaagaggatctggcagcaaatgatatcctggattacaaggatgacgacgataaggtttaaacctaggcgtagcggccgcaaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatgaccgagtacaagcccacggtgcgcctcgccacccgcgacgacgtcccccgggcagtacgcaccctcgccgccgcgttcgccgactaccccgccacgcgccacaccgtcgatccagaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgatgtacaagtaggattcgtcgagggacctaataacttcgtatagcatacattatacgaagttatacatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccatactcttcctttttcaatattattgaagcatttatcagggttattgtctcatgagcggatacatatttgaatgtatttagaaaaataaacaaataggggtcccgcgcacatttccccgaaaagtgccacctgac

SEQ ID NO: 157 - DNA, 8388 bp, Artificial Sequence
  Description of Artificial Sequence Synthetic polynucleotide
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagacatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccggggaattcaccggtgccgccaccatgaagtgggtgaccttcatcagcctgctgttcctgttcagcagcgcctacagcaccgtgaccgtgcccaaggacctgtacgtggtggagtacggcagcaacatgaccatcgagtgcaagttccccgtggagaagcagctggacctggccgccctgatcgtgtactgggagatggaggacaagaacatcatccagttcgtgcacggcgaggaggacctgaaggtgcagcacagcagctaccgccagcgcgcccgcctgctgaaggaccagctgagcctgggcaacgccgccctgcagatcaccgacgtgaagctgcaggacgccggcgtgtaccgctgcatgatcagctacggcggcgccgactacaagcgcatcaccgtgaaggtgaacgccccctacaacaagatcaaccagcgcatcctggtggtggaccccgtgaccagcgagcacgagctgacctgccaggccgagggctaccccaaggccgaggtgatctggaccagcagcgaccaccaggtgctgagcggcaagaccaccaccaccaacagcaagcgcgaggagaagctgttcaacgtgaccagcaccctgcgcatcaacaccaccaccaacgagatcttctactgcaccttccgccgcctggaccccgaggagaaccacaccgccgagctggtgatccccgagctgcccctggcccacccccccaacgagcgcaccggtggcgagatcgccgccctggagcaggagatcgccgccctggagaaggagaacgccgccctggagtgggagatcgccgccctggagcagggcggcggatccgacgacgacgacaagggcagcggctggagccacccccagttcgagaagtgatgagctagccagaaactcatctcagaagaggatctggcagcaaatgatatcctggattacaaggatgacgacgataaggtttaaacctaggcgtagcggccgcaaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatgaccgagtacaagcccacggtgcgcctcgccacccgcgacgacgtcccccgggcagtacgcaccctcgccgccgcgttcgccgactaccccgccacgcgccacaccgtcgatccagaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgatgtacaagtaggattcgtcgagggacctaataacttcgtatagcatacattatacgaagttatacatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccatactcttcctttttcaatattattgaagcatttatcagggttattgtctcatgagcggatacatatttgaatgtatttagaaaaataaacaaataggggtcccgcgcacatttccccgaaaagtgccacctgac

SEQ ID NO: 158 - protein, 18 aa, Homo sapiens
MKWVTFISLLFLFSSAYS

SEQ ID NO: 159 - protein, 5 aa, Artificial Sequence
  Description of Artificial Sequence Synthetic peptide
GGGGS

SEQ ID NO: 160 - protein, 10 aa, Artificial Sequence
  Description of Artificial Sequence Synthetic peptide
GGGGSGGGGS

SEQ ID NO: 161 - protein, 15 aa, Artificial Sequence
  Description of Artificial Sequence Synthetic peptide
GGGGSGGGGSGGGGS

SEQ ID NO: 162 - protein, 20 aa, Artificial Sequence
  Description of Artificial Sequence Synthetic peptide
GGGGSGGGGSGGGGSGGGGS

SEQ ID NO: 163 - protein, 25 aa, Artificial Sequence
  Description of Artificial Sequence Synthetic peptide
GGGGSGGGGSGGGGSGGGGSGGGGS

SEQ ID NO: 164 - protein, 38 aa, Artificial Sequence
  Description of Artificial Sequence Synthetic polypeptide
MDEKTTGWRGGHVVEGLAGELEQLRARLEHHPQGQREP

(19) United States
(12) Patent Application Publication
(10) Pub. No.: US 2020/0048321 A1
(43) Pub. Date: Feb. 13, 2020

(54) PANTIDS FOR TREATMENT OF AUTOIMMUNE DISORDERS

(71) Applicant: ORPHEUS BIOSCIENCE INC., Manassas, VA (US)

(72) Inventors: Cohava GELBER, Nokesville, VA (US); Mark SPEAR, Marshall, VA (US); John ABELES, Boca Raton, FL (US)

(73) Assignee: ORPHEUS BIOSCIENCE INC., Manassas, VA (US)

(21) Appl. No.: 16/497,225

(22) PCT Filed: Mar. 23, 2018
(86) PCT No.: PCT/US2018/024189

§ 371 Date: Sep. 24, 2019

Related U.S. Application Data
(60) Provisional application No. 62/476,371, filed on Mar. 24, 2017.
(60) Provisional application No. 62/549,313, filed on Aug. 23, 2017.
(60) Provisional application No. 62/553,648, filed on Sep. 1, 2017.

(51) Int. Cl. C07K 14/54; A61K 47/65; A61K 47/66; C07K 14/525; C07K 14/52; C07K 14/56; C07K 14/565; C07K 14/57; A61K 47/64; C07K 16/28

(52) U.S. Cl. CPC C07K 14/54; A61K 47/65; A61K 47/66; C07K 14/525; C07K 14/523; A61K 45/06; C07K 14/565; C07K 14/57; A61K 47/6415; C07K 16/2875; C07K 14/56

(57) ABSTRACT

Checkpoint receptors and their cognate ligands are frequently targeted in autoimmune disorders by B and T cells, wherein these adaptive immune responses are likely to significantly contribute to the underlying immunopathology. A novel technology for the clonal elimination of autoreactive B cells that targets checkpoint receptors and their ligands is described herein. One embodiment of this technology is a checkpoint receptor or ligand extracellular domain molecular chimera with an effector domain, which is capable of inducing B cell apoptosis, necrosis, and/or tolerization/anergization: herein, this technology is referred as PantIds (polyclonal anti-idiotypics). In other embodiments, this technology also includes effector molecular chimeras with immunoregulatory cytokines. Novel apoptotic effectors are also described. Methods for the identification of checkpoint receptor/ligand autoreactive B cell responses, construction of PantIds, and their in vitro and in vivo application are also described.

### BACKGROUND OF THE INVENTION

### Normal Tolerogenic Mechanisms

Autoimmune disorders are characterized by the gradual, and often progressive, decline of tolerogenesis and tolerogenic mechanisms that normally preclude adaptive immune responses to endogenous host proteins. During normal B and T cell development, autoreactive cells are eliminated in the bone marrow and thymus, respectively, creating “central tolerance” to host tissues and proteins. For B cells, expression of high-affinity B cell receptors (BCRs) to cell-surface proteins present in the bone marrow, the location of B cell development, results in apoptosis. Additionally, B cells that respond to ubiquitous soluble ligands are deactivated by anergy. For T cells, a similar process occurs in the thymus, the location of T cell development: T cells whose T cell receptor (TCR) responds with high-affinity to self-antigen peptides presented in MHC-I or MHC-II complexes are also deleted by apoptosis. For T cells with intermediate or low-affinity for said peptide-MHC complexes, these cells may develop into regulatory T cells (Tregs), which help maintain peripheral tolerance; alternatively, these cells may become anergic or undergo apoptosis.

Peripheral tolerance refers to a suite of mechanisms that preclude adaptive immune responses to host proteins outside the central immune system. As afore-mentioned, these include centrally generated Treg cells, which help maintain peripheral tolerance by expressing immunosuppressive effectors in response to self-antigen peptide-MHC complexes. The mechanisms of Treg suppression are still being defined, but include the secretion of soluble immunosuppressive effectors and cell-contact-specific immunosuppressors. In the former mechanism, TGF-β, IL-10, adenosine (produced by CD39 and CD73), and IL-35 are secreted from Treg cells to create an immunosuppressive milieu that can prevent T and B cell activation, and create tolerogenic APCs¹. In cell contact mechanisms, CTLA-4, PD-L1, LAG-3, membrane-bound TGF-β, and perforin and granzymes contribute to immunosuppression¹. Also in the periphery, autoreactive T cells can be apoptosed or converted into peripheral Tregs by tolerogenic APCs, such as BTLA⁺ dendritic cells². These peripheral Tregs (pTregs) contributed to peripheral tolerance through many of the mechanism described for central or thymic Tregs (cTregs or tTregs).

An additional mechanism of peripheral tolerance is the general requirement for costimulation for T cell activation. When T cells engage their cognate antigen as peptide-MHC complex, there are two likely outcomes, depending on the presence of costimulation: in the presence of costimulatory agonist, such as CD80 or CD86 binding to T cell-expressed CD28, the T cell becomes activated, resulting in proliferation and the engagement of effector functions; in the alternate case, where costimulation is absent or when T cells receive inhibitory signals in lieu of or in combination with costimulatory signals, the T cells may undergo apoptosis, anergization, or conversion into pTreg. For B cells, a similar costimulation requirement exists for T cell-dependent B cell activation, wherein T cell-expressed CD40L must bind to B cell-expressed CD40 for B cell activation. While these canonical modalities of co-stimulation (e.g. CD28 and CD40) are the best described, additional co-stimulators and co-inhibitors have been recently elucidated: such receptors and their ligands, which cumulatively determine the outcome of antigen engagement, are referred to as immune checkpoint receptor or ligands: currently, these immunologic checkpoints include 15 signaling axes (FIG. 1).

In one example of baseline regulatory autoreactivity towards checkpoint receptors, Andersen et al. (2013) exhibited the presence of CD8 T cells that naturally recognize the immune checkpoint ligand, PD-L1⁷. These anti-PD-L1 cytotoxic T lymphocyte (CTL) responses were observed in healthy patients and, to a greater extent, in patients with renal cell carcinoma or malignant melanoma: it was conjectured that naturally occurring anti-PD-L1 CTL respond to the high-level PD-L1 expression, amid inflammation, in the tumor microenvironment, leading to increased anti-PD-L1 CTL responses in cancer patients⁷. The authors also noted that these naturally-occurring anti-PD-L1 CD8 T cells may play an immunoregulatory role in healthy patients by modulating the frequency of PD-L1-expressing cells: for instance, anti-PD-L1 CTLs may reduce autoimmunity by eliminating PD-L1-expressing APCs. This same group observed the presence of anti-PD-L1 Th17 cells, an inflammatory subset of CD4 T cells: these cells were also postulated to regulate both baseline immunity and anti-cancer immunity, as in the case of anti-PD-L1 CTLs⁸.

### BRIEF SUMMARY OF THE INVENTION

The inventions described and claimed herein have many attributes and aspects including, but not limited to, those set forth or described or referenced in this Brief Summary. It is not intended to be all-inclusive and the inventions described and claimed herein are not limited to or by the features or embodiments identified in this Brief Summary, which is included for purposes of illustration only and not restriction.

In one aspect, the technology described herein relates to PantIds, the production of PantIds, and use of the PantIds for the specific targeting of autoreactive B cells whose cognate antigens correspond to checkpoint receptors or their ligands: these autoreactive B cells are contributory and, perhaps, etiological in the onset and progression of autoimmune diseases. In one aspect, the PantId may be a molecular chimera comprising two to five components, for example two, three, four, or five components, for example, (1) a first component selected from a checkpoint ligand, receptor, or immunoregulatory cytokine; and (2) a second component comprising an effector, where the effector elicits leukocyte apoptosis, necrosis, tolerization, or anergization. The PantId may also comprise a linker between each of the two to five, for example two, three, four, or five, components, to provide flexibility to the molecular chimera. The PantId may also comprise additional effectors and/or a homodimerization, heterodimerization, trimerization, tetramerization, or oligomerization domain.

In some aspects this disclosure features methods and vectors for targeting autoreactive B cells in patients with a PantId comprising a known antigen and an antibody or fragment thereof. For example, the PantId may comprise an Fc (fragment crystallizable) portion of an Ab—the Fc comprise two heavy chains that each contain two or three constant domains depending on the class of the antibody. Humans have five different classes of Fc receptors (FcR)—one for each class of antibody. FcR haplotypes or genetic variants have also been reported. Interactions of an Fc domain with FcRs and other subclsses of antibodies mediates recruitment of other immunological cells and the type of cell recruited. Hence, the ability to engineer Fc domains that bind to selected FcRs and/or other classes and subclasses of immunoglobulins, and recruit only desired types of immune cells can be important for therapy. In one aspect, the Fc region of IgG can be engineered to bind to the transmembrane isoforms of IgD, IgM, IgG1-4, etc., on autoreactive B cells. When B cell receptors binds to the autoantigen-Fc fusion protein, the B cells are targeted for cytolysis. In other aspects, the PantIds of this disclosure may exclude Fc domains.

In some aspects of this disclosure the PantId components target the same cell. In some aspects of this disclosure the PantId components target the same autoreactive B cell. In some aspects, a PantId comprises a molecular chimera comprising the extracellular domain of a checkpoint receptor or its cognate ligand, and an effector or effector domain, where the effector or effector domain promotes B cell apoptosis, necrosis, or tolerization/anergization. In some aspects, treatment with a PantId leads to clonal deletion of autoreactive B cells. For example, in one embodiment, a molecular chimera comprises a PD-L1 extracellular domain and a FasL extracellular domain, which mediates polyclonal anti-PD-L1 autoreactive B cell apoptosis. In this embodiment, administration of the PantId leads to clonal deletion of the anti-PD-L1 autoreactive B cells. In some embodiments the PantIds of this invention are particle-free.

In one aspect, therapeutic compositions comprising a PantId are useful for the treatment or amelioration of autoimmune diseases characterized by autoreactive B cells which exhibit responsiveness to immunologic checkpoint receptors, or their ligands, or immunoregulatory cytokines. In one aspect, these PantIds will target autoreactive B cells through their B cell receptor (BCR), resulting in clonal deletion. In one aspect, the clonal deletion of anti-checkpoint protein autoreactive B cells will result in significant mitigation of autoimmune-associated inflammation, morbidity, and mortality. In some aspects, administration of the PantId will result in clinical amelioration of autoimmune disease symptoms associated with the central role of autoreactive B cells in underlying immunopathology. More specifically, for autoimmune diseases and disorders in which these anti-checkpoint autoreactive B cells play a pivotal role in autoimmune diseases, clonal deletion of the autoreactive B cells by the PantIds will result in more apparent clinical benefits than other therapeutics targeting downstream events.

In another aspect of this disclosure the PantId may include or exclude a portion of the immunogenic therapeutic drug antibody comprising the epitope on the theraepeutic drug antibody to which the autoantibodies bind. In this aspect, the PantId comprise cognate antigens from therapeutic antibodies are useful in treating immunogenic reactions to therapeutic antibodies.

When anti-checkpoint protein T cells play a role in baseline immunoregulation, their dysregulation may contribute to autoimmunity. For example, one role of checkpoint receptors and ligands described herein is the role of checkpoint proteins as autoantigens themselves. In this capacity, autoantibodies and T cell responses towards immunologic checkpoint proteins can blockade checkpoint co-inhibitors, agonize checkpoint co-stimulators, or dysregulate delicately balanced cytokine networks. These immune responses exacerbate, potentiate, and possibly even instigate autoimmune pathologies by promoting unregulated T and B cell activation.

In one aspect, this disclosure relates to compositions and methods for treating or ameliorating autoimmune diseases and disorders by countering autoreactive adaptive immune responses toward immunologic checkpoint proteins which are clinically contributory to autoimmunity. As one non-limiting example, a sudden increase in anti-checkpoint proteins may eliminate checkpoint-positive Tregs, for example an anti-PD-L1 CTL and Th17 responses could eliminate PD-L1-positive Tregs, undermining a pivotal component of peripheral tolerance. The PD-L1 PantIds of this disclosure will, in one aspect, be useful in restoring tolerance.

This disclosure also relates to methods for the detection and identification of autoimmune responses to checkpoint receptors, their ligands, and immunoregulatory cytokines for the following purposes: (1) to determine the prevalence of said responses in well-characterized autoimmune disorders (i.e. systemic lupus erythematosus); (2) to further define and expand a list of candidate PantId molecular chimeras partners, with an emphasis on checkpoint receptors, their ligands, and immunoregulatory cytokines; (3) and the tailoring of PantId therapies for patients, wherein a subset of PantIds may be administered based on the immunoreactivity profile of the patient's serum.

Pursuant to this, methods for screening patient serum, cloning of PantIds, and PantId administration in vitro, in in vivo models, and in patients is described. In one aspect, this disclosure relates to methods of screening patient serum comprising contacting a patient sample with a panel of two or more checkpoint proteins, checkpoint receptors, their ligands, and immunoregulatory cytokines or portions thereof, to form complexes with auto-antibodies in the patient sample; and detecting any complexes. In some embodiments the panel will comprise two, three, four, five, six, seven, eight, nine, or ten, or more checkpoint proteins, checkpoint receptors, their ligands, and immunoregulatory cytokines or portions or epitopes thereof. In some embodiments the panel will comprise up to or over 9,000 human proteins, including checkpoint proteins, checkpoint receptors, their ligands, and immunoregulatory cytokines and other proteins. In some embodiments the profile is obtained using reverse phase protein microarray (RPMA). In some embodiments, PantId therapies are tailored and administed to patients based on the patient's immunoreactivity profile. In some embodiments, the panel of checkpoint proteins, checkpoint receptors, their ligands, and immunoregulatory cytokines or portions thereof may comprise labeled polypeptides or portions thereof, or labeled anti-human antibodies, and labeled complexes are detected to obtain the patient's immunoreactivity profile, as described further herein. The label may, in some embodiments be, e.g., an enzyme, chemiluminescent, fluorescent, or nanoparticle label.

Detection of autoimmune responses to checkpoint receptors, their ligands, and immunoregulatory cytokines will determine whether pervasive anti-checkpoint protein T and B cell autoreactivity contributes to, and/or is entirely responsible for, systemic autoimmunity. This determination may radically change current paradigms regarding autoimmune disorder genesis and treatment, using the PantIds of this disclosure.

As an example, anti-immunologic checkpoint responses have been observed in patients with autoimmune disorders. As exemplified herein, reverse phase protein microarray (RPMA) studies have detected anti-PD-L1 and anti-IL-10 responses in an autoimmune patient's serum: contrastingly, these responses were absent in the healthy control serum. In another example of this phenomenon, it was surprisingly detected that 8.2% of patients with systemic lupus erythematosus (SLE), 18.8% of patients with rheumatoid arthritis, 3.1% of patients with systemic sclerosis, 31.8% of patients with Behcet's disease, 13.3% of patients with Sjögren's syndrome, while 0% of healthy donors had detectable autoantibody responses to the immunosuppressive checkpoint receptor, CTLA-4⁹. Furthermore, these CTLA-4 autoantibodies are contributory to the immunopathology, as they negatively correlated with uveitis in Behcet's disease⁹, and promoted T cell proliferation in vitrol¹⁰.

In another aspect, this disclosure relates to methods for the production of PantIds. Such a method may include cloning of a protein/peptide molecular chimera comprising (1) a first domain selected from: a checkpoint receptor, ligand, or immunoregulatory cytokine or any portion thereof that binds to the autoreactive B cell, including any extracellular domain or epitope of the a checkpoint receptor, ligand, or immunoregulatory cytokine; and (2) a second domain comprising an effector or any portion thereof, or a homodimerization, heterodimerization, trimerization, tetramerization, or oligomerization domain. Cloning of the molecular chimera PantId may use any nucleic acid expression system or combination of expression systems, with or without IRES elements or P2A//T2A picomaviral slip sites or alternative polyprotein/polycistron expression motifs and modalities. Alternatively, a molecular chimera may be produced by chemically linking the two or more components. For example, in one aspect, an effector or effector molecular chimera is covalently linked by chemical coupling reagent to an immunological checkpoint receptor, ligand, or immunoregulatory cytokine.

In one aspect, this disclosure relates to methods for the introduction of PantIds in cell culture, animal models, and humans as recombinant proteins, including by viral and non-viral protein transduction. The present disclosure also includes methods for therapeutic efficacy or bioactivity assessment and quantification, including, but not limited to, cell viability assays, cell death assays, cell metabolisms assays, cytostatic assays, cell proliferation assays, targeted cell killing assays, immune cell killing assays, flow cytometric assays, Western blot assays, cytokine ELISAs and Western blot assays, whole blood workup assays, leukocyte counts, HPLC and mass spectrometric assays, ELISpot assays, fluorescent and chemiluminescent-linked immunosorbent assays, in vivo imaging, etc.

### BRIEF DESCRIPTION OF FIGURES

FIG. 1: Depiction of immunologic checkpoint receptors and their ligands. T cells receive a primary signal, depicted as “Signal 1,” when MHC-I or MHC-II:peptide complexes bind the T cell receptor (TCR). This signal primes the T cells for activation, anergy, or apoptosis. However, the fate of the T cell is ultimately determined by specific combinations of stimulatory and inhibitory immunological checkpoint receptor signaling, which can bias the T cell response towards one of these 3 outcomes.

FIGS. 2 A and 2 B: Two instantiations of PantId technology. FIG. 2A provides a DNA fragment map of a PD-L1-FasL covalent molecular chimera. FIG. 2B provides maps of two DNA fragments separately encoding PD-L1 and FasL as molecular chimeras with cognate heterodimerization domains. In some embodiments the PantIds from FIG. 2B are co-transfected into mammalian cells after cloning into expression constructs for the production of PD-L1-CC-BN₄:FasL-CC-AN₄ heterodimers. This achieves the same therapeutic functionality of (A), but with simpler gene synthesis, cloning, and in vitro characterization.

FIGS. 3 A and 3 B: Plasmid maps of PD-L1-FasL molecular chimera fragment, and cloned into lentivector pLenti-C-Myc-DDK-IRES-Puro. FIG. 3 A depicts a PD-L1-FasL molecular chimera fragment with terminal restriction sites, which allow cloning into pLenti-C-Myc-DDK-IRES-Puro. FIG. 3 B provides a plasmid map of a final pLenti-C-PD-L1-FasL-IRES-Puro vector, which would be used as both an expression vector, and as a lentivector for lentiviral transduction of producer cells.

FIG. 4: Amino acid sequences.

FIG. 5: Depiction of mechanism of action of an PantId comprising an autoantigen-Fc. Autoantigen IgG-fusion proteins are represented by an IL-2Rβ ECD-IgG1 Fc fusion that neutralizes circulating autoantibodies to IL-2 RP. Also shown is the binding of the autoantigen Fc fusion protein to the autoantibody-secreting B cell's BCR (B cell antigen receptor), resulting in ADCC, complement activation, and autoreactive B cell apoptosis.

FIG. 6: Plasmid maps of pLenti-C-Myc/DDK-IRES-Puro into which the PantIds are cloned into. Shown is the SIN 3 LTR, 5 LTR, Rev-Response Element (RRE), central polypurine tract (cPPT), internal ribosome entry site (IRES), Puromycin Resistance gene (PuroR), and the Woodchuck Hepatitis Virus (WHP) Posttranscriptional Regulatory Element (WPRE). This sequence corresponds to Sequence ID 00132.

FIG. 7: Plasmid maps of CTLA-4-hIgG1 Fc cloned into vector pLenti-C-Myc/DDK-IRES-Puro. Shown is the extracellular domain.n (ECD) of human CTLA-4-Fc fused to human IgG1 Hinge, CH2, and CH3 regions. This sequence is cloned into the 5 EcoRI and 3 BamHI sites of the pLenti-C-Myc/DDK-IRES-Puro multiple cloning site (MCS). This sequences corresponds to Sequence ID 00133.

FIGS. 8 A and 8 B: Plasmid maps of PD-L1 (8 A) and FasL (8 B) PantId heterodimers cloned into pLenti-C-Myc/DDK-IRES-Puro. These sequences correspond to Sequence ID 00134 and 00135, respectively.

FIG. 9: FIG. 9 shows a bar graph of the titers of CTLA-4 PantId in the supernatant of HEK293T cells contacted with PantId constructs and control constructs. The bars labeled Clones 1-4 (see the labels on the Y-axis) show the CTLA-4 PantId titers from supernatants from HEK293T cells transfected with each of the four pLenti-C-CTLA4-hIgG₁ Fc-IRES-puro clones; two negative controls included titers from cells contacted with a vector without a CTLA4-hIgG₁ insert, and cells contacted with culture medium only. The titer in supernatant from vLenti-C-CTLA-4-hIgG₁ Fc-IRES-puro transduced HEK293T cells is also shown.

FIG. 10: FIG. 10 shows a Western Blot demonstrating that CTLA-4-hFc PantId adopts a homodimeric structure. Results are shown for CTLA-4-hFc. pLenti-C-CTLA-4-hIgG1 FC-IRES-Puro clones 1-4 were transfected into HEK293T cells and the supernatants were analyzed in the presence or absence of a reducing agent. The first four lanes from the left identify the samples from each of the four clones, exposed to a reducing agent. The next four lanes are samples from each of the four clones, identifying the oligomer, homodimer, and monomer structures of the CTLA-4-hFc PantIds in the absence of the reducing agent. Empty parental pLenti-C-Myc/DDK-IRES-Puro vector is denoted by “E.” Additionally, in the reduced samples, the CTLA-4-hFc monomer exhibits the predicted molecular mass of 43 kDa. Higher molecular weight bands correspond to oligomers and glycovariants thereof.

FIG. 11 is an immunoblot showing first components of PantIds binding to anti-human CTLA-4, PD-1, and PD-L1 antibodies. Purified CTLA-4-Fc, PD-1-CCAN4, and PD-L1-CCAN4 first components of PantIds were analyzed by SDS gel electrophoresis and transferred to nitrocellulose membranes. The left-hand panel shows a nitrocellulose membrane probed with mouse anti-human CTLA-4. The left-hand center panel shows a similar nitrocellulose membrane probed only with goat anti-mouse secondary antibody. The right-hand center panel shows a similar nitrocellulose membrane probed with anti-human PD-1 antibody. The right-hand panel shows a similar nitrocellulose membrane probed with anti-human PD-L1 antibody.

FIG. 12 depicts the results of an experiment showing that PD-1-CCAN4 first component of a PantId specifically neutralized the binding of mouse anti-human PD-1 to recombinant human PD-1 protein.

FIG. 13 depicts the results of an experiment showing that PD-1-CCAN4 first component of a PantId specifically neutralized the binding of mouse anti-human PD-1 to recombinant human PD-1 protein. PD-1-CCAN4 first component of a PantId neutralized 1 μg/ml anti-human PD-1 with an IC₅₀ of 136 ng or 31.8 nM, with PD-1-CCAN4 first component of a PantId exhibiting an observed molecular weight in SDS-PAGE of 43 kDa.

FIG. 14 shows specific binding of reduced and non-reduced CTLA-4-Fc PantId by anti-human CTLA-4 antibody.

FIG. 15 shows the purification of PD-L1-CCAN4-SBP polypeptide by Strep-Tactin Resin.

FIG. 16 shows the purification of PD-L1-CCAN4-SBP polypeptide by Strep-Tactin Resin and the expression of FasL-CCBN4-SBP and TRAIL-CCBN4-SBP second components of PantIds in CHO cells.

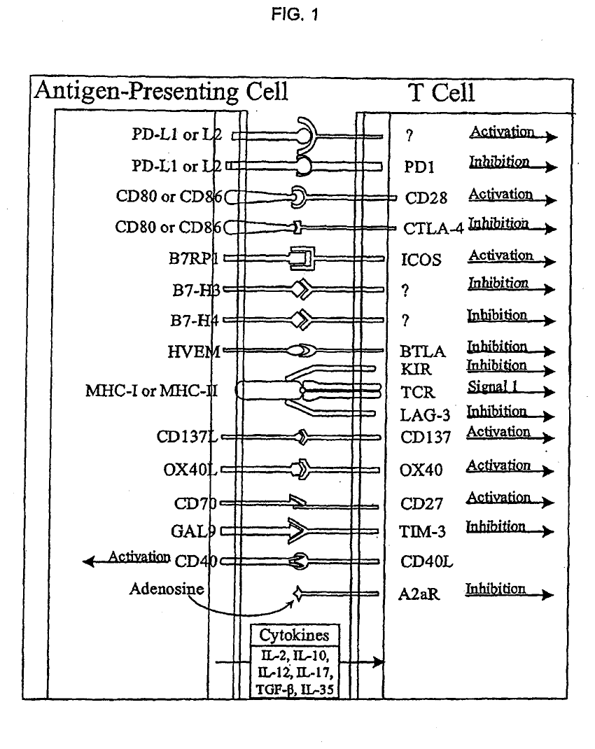

### DETAILED DESCRIPTION OF THE INVENTION

In one embodiment, this disclosure relates use of PantIda as therapeutics for the treatment of autoimmune diseases, characterized by autoreactive B cells which exhibit responsiveness to immunologic checkpoint receptors, or their ligands, or immunoregulatory cytokines.

Although the mechanism of self-tolerance and autoimmunity is poorly understood for most autoimmune diseases, there are rare examples where the mechanism of initial tolerance is well characterized. For example, in Coxscackievirus B-associated myocarditis³ the initial viral infection is followed by inflammatory sequelae involving the myocardium and pericardium: this is associated with mononuclear cell infiltration, antibodies to cardiac actin and myosin, and associated CD4 T cells responses, which promote the clinical presentation of myocarditis³. In this example, Coxscackievirus-associated antigens molecularly mimic cardiac myosin and actin, and the resultant T and B cell responses continue in the absence of viral infection due to the capacity of cardiac myosin and actin to activate these autoreactive T and B cells. Similarly, in streptococcal-induced rheumatic heart disease, adaptive immune responses to streptococcal M protein cross-react with cardiac myosin and actin, resulting in a similar immunopathology^{4,5}. Of note, these pathogen-associated autoimmune conditions are typically acute, and therefore, as recognized herein, other underlying predispositions towards autoimmunity likely coincide with such instigating stimuli to induce chronic clinical autoimmune diseases.

As such, during the initial breakdown in self-tolerance, molecular mimicry between a pathogen's protein and a host protein can promote T and B cell reactivity to host proteins. More generally, the presence of alternate inflammatory stimuli in endogenous host tissues can result in aberrant T and B cell responses to these tissues. These inflammatory stimuli can lead to the expression of immunologic checkpoint co-stimulators that bypass one of the pivotal mechanisms of peripheral tolerance—the requirement for co-stimulation. More broadly, the initial inflammatory state that leads to checkpoint co-stimulator expression is not necessarily pathogen-derived, and could be caused by commensal bacteria, tissue injury, radiation, or chemical exposure, which can promote inflammation through pathogen-associated molecular pattern receptors (PAMPs) or damage-associated molecular pattern receptors (DAMPs). Alternatively, exposures to haptens, which covalently couple to host proteins and render them immunogenic, could lead to autoimmune responses in the presence of co-stimulation.

Overlaid on these mechanisms of tolerance breaking are monogenic and polygenic predispositions towards autoimmunity, which include, but are not limited to, the following: (1) specific HLA haplotypes, which are associated with efficacious MHC presentation of particular host peptides, thus predisposing the host to T cell responses to these peptides; (2) genetic or epigenetic dysregulation of immunologic checkpoint receptor or ligand expression or function, which can create imbalances that bias the adaptive immune system towards systemic activation; and (3) non-checkpoint protein genetic mutations that facilitate chronic inflammation (e.g. tight junction protein mutations, which can promote chronic exposure to commensal bacteria and chronic inflammation). When these underlying genetic predispositions to autoimmunity combine with one of the afore-mentioned instigating stimuli, acute autoimmunity can lead to chronic autoimmunity, morbidity, and mortality.

In other contexts, the administration of checkpoint costimulatory agonists, or checkpoint co-inhibitor antagonists, for anti-tumor or anti-viral therapy can promote opportunistic autoimmune disorders by undermining central and peripheral tolerogenic mechanisms: in these instances, after therapeutic administration, the patient presents immune-related adverse events (IRAEs) due to systemic immunological disinhibition⁶. These IRAEs are frequent, occurring in 90% of patients receiving anti-CTLA-4 antibodies and 70% of patients receiving PD-1/PD-L1 blockade antibodies⁶. While most IRAEs are graded as I-II—mild symptoms, primarily affecting the skin and gastrointestinal tract—more severe grade III-V symptoms are non-uncommon, affecting 1-10% of patients⁶. The management, chronic effects, and IRAE persistence post-treatment are still being characterized, due to the novelty of checkpoint blockade therapies; as such, it is unclear whether these IRAEs comprise a new category of systemic, chronic autoimmune disease.

As previously noted, checkpoint receptors centrally contribute to autoimmunity by licensing T and B cells to respond to host antigens in genetically predisposed populations. In these instances, DAMP and PAMP receptor signaling, associated with inflammation, drives the expression of inflammatory cytokines, such as IL-1β, IL-6, IL-12, and TNF-α: in combination, these promote checkpoint receptor expression, including CD80/CD86 and CD40L, thus eliminating the requirement for co-stimulation necessary for peripheral tolerance. Thereafter, B and T cells become activated, proliferate, and exhibit immunopathological effector functions that contribute to the clinical manifestations of autoimmunity, such as the following: (1) autoantibody production by B cells; (2) autoantibody-mediated cell killing and immune complex formation; (3) cytokine-associated inflammation and inflammation-associated tissue damage mediated by activated innate immune cells, damaged host tissues, and CD4 T cells; and (4) targeted host cell killing by CD8 T cells. Alongside these phenomena is a gradual broadening of the adaptive immune response, from one epitope on one antigen, to multiple epitopes on one antigen, to multiple antigens—termed epitope and antigen spreading, respectively. This broadly coincides with a gradual decline of tolerance and functional tolerogenic mechanisms.

In one embodiment, this disclosure relates to PantIds and their use as a therapeutic for the treatment of autoimmune diseases, characterized by autoreactive B cells which exhibit responsiveness to immunologic checkpoint receptors, or their ligands, or immunoregulatory cytokines. In some embodiments, the PantId comprises two to five proteins, domains, or peptides. For example, in some embodiments the PantId is a molecular chimera comprising two or more components which may comprise, in some embodiments at least (1) a first component selected from a checkpoint ligand, receptor, or immunoregulatory cytokine; and (2) a second component selected from an effector, where the effector elicits leukocyte apoptosis, necrosis, tolerization, or anergization. The molecular chimeras may also comprise additional effectors and/or a homodimerization, heterodimerization, trimerization, tetramerization, or oligomerization domain. The first component of the Pantid binds to a ligand and elicits signaling within leukocytes or lymphoid tissue-associated cells, e.g., autoreactive B cells. The Pantid may also comprise a linker between the two or more components or domains. In some aspects of this disclosure the PantId components target the same cell. In some aspects of this disclosure the PantId components target the same autoreactive B cell. In some embodiments the PantIds of this disclosure are particle-free, e.g., the PantIds do not comprise a microparticle, nanoparticle or other particle carrier or bead.

The linker can be a reagent, molecule or macromolecule that connects the first component and the second component such that a) the PantId is stable under physiological conditions; b) the connection between the linker and the PantId does not alter the ability of the PantId to bind to its target.

In one embodiment, a linker can be a peptide bond. The PantId can be a fusion polypeptide comprising one or more amino acid segments from the first component and one or more amino acid segments from the second component. The amino acid segments of the first component can be contiguous with the amino acid segments of the second component or they can be separated by amino acids inserted as a structural spacer. A spacer segment can be one or more amino acids. The one or more amino acids can include amino acids that are the same or that are different. Also encompassed are nucleic acids comprising a nucleotide sequence that encodes the PantId.

In another embodiment, the first component and second component can be obtained separately, either through chemical synthesis or synthesis in vivo, purified and then linked non-covalently or covalently. The non-covalent linkage can be for example, an ionic bond. The covalent linkage can be through a chemical cross-linking agent, for example, a homobifunctional cross-linking reagent or a heterobifunctional cross-linking reagent. In another embodiment, the first component and the second component can be connected through a linking polymer, including, for example, linear or branched polymers or co-polymers (e.g., polyalkylene, poly(ethylene-lysine), polymethacrylate, polyamino acids, poly- or oligosaccharides, or dendrimers).

The first component and the second component specifically bind their respective targets. In general, components that specifically bind a target exhibit a threshold level of binding activity, and/or do not significantly cross-react with related target molecules. The binding affinity of a component can be determined, for example, by Scatchard analysis. For example, a first component or a second component can bind to its respective target with at least 1.5-fold, 2-fold, 5-fold, 10-fold, 100-fold, 10³-fold, 10⁴-fold, 10⁵-fold, 10⁶-fold or greater affinity for the target than for a closely related or unrelated target. A first component or a second component can bind its target with high affinity (10⁻⁴M or less, 10⁻⁷M or less, 10⁻⁹M or less, or with subnanomolar affinity (0.9, 0.8, 0.7, 0.6, 0.5, 0.4, 0.3, 0.2, 0.1 nM or even less). The first component or the second component can also be described or specified in terms of their binding affinity to a target, for example, binding affinities include those with a Kd less than 5×10⁻²M, 10⁻²M, 5×10⁻³M, 10⁻³M, 5×10⁻⁴M, 10⁴M, 5×10⁻⁵M, 10⁻⁵M, 5×10⁻⁶M, 10⁻⁶M, 5×10⁻⁷M, 10⁻⁷M, 5×10⁻⁸M, 10⁻⁸M, 5×10⁻⁹M, 10⁻⁹M, 5×10⁻¹⁰M, 10⁻¹⁰M, 5×10⁻¹¹M, 10⁻¹¹M, 5×10⁻¹²M, 10⁻¹²M, 5×10⁻¹³M, 10⁻¹³M, 5 ×10⁻¹⁴M, 5×10⁻¹⁵M, or 10⁻¹⁵M, or less.

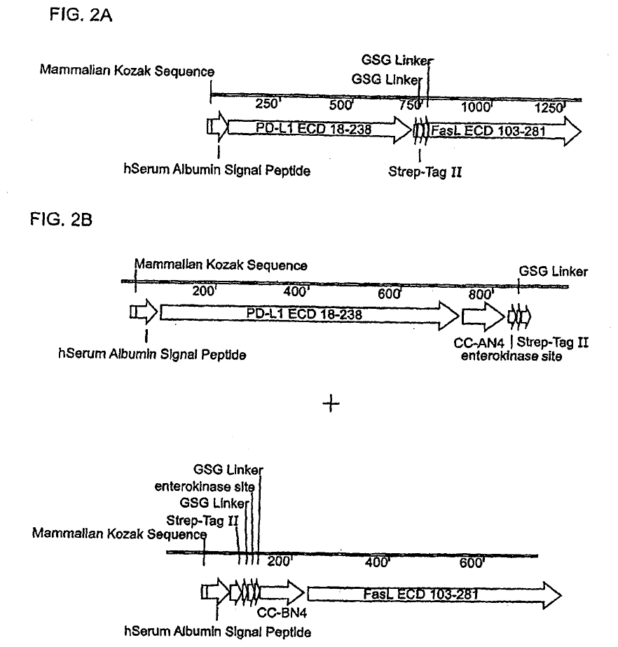

In one embodiment the chimera comprises the extracellular domain of PD-L1 and an apoptosis-inducing FasL extracellular domain. In one instantiation, the extracellular domain of PD-L1 is cloned as a molecular chimera, with the apoptosis-inducing FasL extracellular domain: upon binding of anti-PD-L1 autoreactive B cells through their BCR, FasL engagement of B cell-expressed Fas promotes B cell apoptosis and clonal deletion of this autoreactive clone (FIG. 2A). In this embodiment, one or more PantIds, comprising multiple checkpoint receptor, ligand, or immunoregulatory-effector molecular chimeras with one or more effector classes, are administered intravenously in animal models or human patients to elicit therapeutic effects.

In vitro, PantIds are added to the culture supernatant to determine in vitro effects.

In some embodiments, the molecular chimera comprises a checkpoint ligand, receptor, or immunoregulatory cytokine and a heterodimerization domain, such as described in Thomas et al. 2013¹¹, or a homodimerization domain, a trimerization domain, a tetramerization domain such as described in Mittl et al. 2000¹² (Sequence 131). In some embodiments a cognate heterodimerization domain is also expressed as a molecular chimera with any effector disclosed herein, for example, FasL. When cloned and co-expressed, for example, a molecular chimera of a PD-L1 extracellular domain and a heterodimerization domain CC-AN₄ (Sequence 129) allows directed assembly with the cognate heterodimerization domain, for example, CC-BN₄¹¹ (Sequence 130), which, in some embodiments is expressed as a molecular chimera with an effector (e.g. FasL). As such, assembly of a functional therapeutic—PD-L1-FasL, in this example—is achieved post-translationally (FIG. 2B). This method of PantId construction reduces the gene synthesis and cloning costs of PantIds, and facilitates the in vitro efficacy screening of effector or effector combinations. This methodology will be applied during PantId optimization, as effector and checkpoint protein synergism can be easily identified.

The effector, as used in the first and second embodiments, or any other embodiments disclosed herein may include multiple classes of proteins, domains, peptides, lipids, glycans, and chemicals, as well as complexes and molecular chimeras thereof, as set forth in non-limiting examples that follow.

For example, in some embodiments, the effector component of the PantId can be selected from or may exclude death receptor ligands, comprising CD95L (a.k.a. FasL, Sequence 001), TRAIL (a.k.a. Apo2L, Sequence 002), and TWEAK (a.k.a. Tumor necrosis factor ligand superfamily member 12, Sequence 003) of the effector class of PantIds. In some embodiments, the effector may include or exclude any other member of the TNF receptor superfamily ligands including, but not limited to, OX40L (Sequence 004), TNF-α (Sequence 005), Lymphotoxin-β (a.k.a. TNF-C, Sequence 006) and its binding partner Lymphotoxin-α (a.k.a. TNF-β, Sequence 007), CD154 (a.k.a. CD40L, Sequence 008), LIGHT (a.k.a. CD258 Sequence 009), CD70 (Sequence 010), CD153 (Sequence 011), 4-1BBL (a.k.a. CD137L, tumor necrosis factor (ligand) superfamily, member 9, (Sequence 012), RANKL (a.k.a. CD254, Sequence 013), APRIL (Sequence 014), Nerve growth factor ligands (e.g. NGF Sequence 015, BDNF (Sequence 016), NT-3 (Sequence 017), and NT-4 (Sequence 018), BAFF (Sequence 019), GITR ligand (Sequence 020), TL1A (Sequence 021), and EDA-A2 (Sequence 022).

In some embodiments, the effector component of the PantId is selected from any of the following, or its ligand, or may exclude any of the following, or its ligand: (a) Leukocyte-associated immunoglobulin-like receptor 1 (LAIR-1), an inhibitory receptor found on peripheral mononuclear cells, including NK cells, T cells, and B cells; (b) Sialic acid-binding immunoglobulin-type lectins (Siglecs), for example, Siglec-1 (CD169), Siglec-2 (CD22), Siglec-3 (CD33), Siglec-4 (Myelin-associated glycoprotein), Siglec-10, CD33-related Siglecs (Siglecs 5-12); (c) Fc-gamma receptors, for example FcγRI, FcγRII, FcγRIII; (d) Leukocyte immunoglobulin-like receptor subfamily B member 3 (LILRB3), PIR-B, ILT-2, ILT-5; (e) CD5, CD66a, CD72.

In some embodiments the effector component of the PantId may be selected from or may exclude: (a) Modified bacterial toxins, including A-B toxins and autotransporters, for the delivery of cytotoxic effectors intracellularly, wherein said cytotoxic effector may be a caspase, bacterial toxin, or other enzyme; (b) A cytotoxic or cytostatic agent small-molecule of less than 10,000 Daltons, such as microtubule or actin cytoskeletal modulators, inhibitors of DNA replication, ribosomal inhibitors, inhibitors of RNA synthesis, radionuclides and coordination complexes thereof, etc.; (c) An NK activating receptor ligand, including: MICA (Sequence 023) and MICB (Sequence 024), which bind NKG2D; ULBP1-6 (Sequences 025-030), Rae-1 (Sequence 031), MULTI (Sequence 032), H60 (Sequence 033), which bind to NKG2D; the DNAM-1 ligands, CD155 (Sequence 034) and CD112 (Sequence 035); B7-H6 (Sequence 036) and BAT3 (Sequence 037); which bind to NKp30; and CD27, which binds CD70; (d) An immunomodulatory cytokine, such as IL-1β, IL-6, IL-7, IL-10, IL-12, IL-21, IL-35, TGF-β, TNF-α, type I interferons, type II interferons, type III interferons, canonical chemokines (e.g. CC, CXC, C, and CX₃C classes), and non-canonical chemotactic or chemokinetic agents (e.g. Slit1, 2, and 3); or (e) An Fc domain of human, murine, porcine, or canine immunoglobulins, including IgA, IgM, IgG, IgD, IgE, and their subclasses. In some embodiments the Fc can increase the bioavailability and/or half-life of the PantId. In some embodiments the PantId effector component may exclude any of the Fc domains listed above.

In one embodiment of this disclosure, the checkpoint receptor, ligand, or immunoregulatory cytokine in the PantId is oligomerized in the absence of an effector. In one instantiation of this, PD-L1 oligomers are therapeutically applied for the elimination of anti-PD-L1 autoreactive B cells by activation-induced cell death (AICD). In this embodiment, the first component of the molecular chimera of the PantId selected from the checkpoint receptor, ligand, and immunoregulatory cytokine, is cloned with a homodimerization, heterodimerization, trimerization, tetramerization, or oligomerization domain, in order to achieve oligomerization.

In one embodiment, the immunological checkpoint receptor is an intracellular, transmembrane, or membrane-associated protein that binds to a ligand and/or that binds to and elicits signaling within leukocytes or lymphoid tissue-associated cells, such as autoreactive B cells. In some embodiments, the signaling within leukocytes or lymphoid tissue-associated cells mediates an immunomodulatory effect by an NF-κB, NFAT, JAK-STAT, PI-3K, PLC, PKC, cAMP-PKA, cGMP-PKG, MAPK, caspase, SMAD, Rho-family GTPase, tyrosine kinase or phosphatase, lipid kinase or phosphatase pathway; or by other signaling pathways in T and B cells, natural killer (NK) cells, dendritic cells (DCs), natural killer T (NKT) cells, granulocytes (neutrophils, basophils, eosinophils, and mast cells), monocytes, macrophages, or lymphoid tissue-associated cells of diverse origins and phenotypes (e.g. follicular dendritic cells).

In any of the embodiments herein, the checkpoint receptor may be selected from or may exclude any of the following proteins, as well as any active portion, peptide or epitope thereof that binds to and/or elicits signaling within leukocytes or lymphoid tissue-associated cells, e.g., autoreactive B and/or T cells autoreactive B cells or T cells: PD-1 (Sequence 038); CD28 (Sequence 039); CTLA-4 (Sequence 040); ICOS (Sequence 041); BTLA (Sequence 042); KIR (Killer immunoglobulin receptors), including: KIR2DL1 (Sequence 043), KIR2DL2 (Sequence 044), KIR2DL3 (Sequence 045), KIR2DL4 (Sequence 046), KIR2DL5A (Sequence 047), KIR2DL5B (Sequence 048), KIR2DS1 (Sequence 049), KIR2DS2 (Sequence 050), KIR2DS3 (Sequence 051), KIR2DS4 (Sequence 052), KIR2DS5 (Sequence 053), KIR3DL2 (Sequence 054), KIR3DL3 (Sequence 055), and KIR3DS1 (Sequence 056); LAG-3 (Sequence 057); CD137 (Sequence 058); OX40 (Sequence 059); CD27 (Sequence 060); CD40 (Sequence 061); TIM-3 (Sequence 062) and other T-cell immunoglobulin and 1-domain containing (TIM) receptors, including TIM-1 (Sequence 063), TIM-2 (Sequence 064), and TIM-4 (Sequence 065); A2Ar (Sequence 066); And And any transmembrane, peripheral membrane, membrane-associated, or cytosolic protein containing an ITAM (immunoreceptor tyrosine-based activating motif, Sequence 067), ITIM (immunoreceptor tyrosine-based inhibitory motif, Sequence 068), or ITSM (immunoreceptor tyrosine-based switch motif, Sequence 069) motif, domain, or peptide, such as CD244 (2B4, Sequence 070)) and TIGIT receptor (Sequence 071). In some embodiments, when the checkpoint receptor is CTLA-4, CD27, ICOS, or portions thereof, the effector is not FasL, TRAIL, TWEAK, or portions thereof. In some embodiments, the checkpoint receptor is not CTLA-4.

In one embodiment, the PantId molecule comprises an immunological checkpoint ligand, which may be a protein, domain or peptide capable of eliciting signaling in an immunological checkpoint receptor, and/or that binds to and elicits signaling within leukocytes or lymphoid tissue-associated cells, such as autoreactive B cells. In some embodiments, the signaling is reverse signaling by which checkpoint receptor binding to checkpoint ligand is associated with ligand-expressing cell signaling, or where the ligand exhibits properties of both a receptor or ligand, the commonly used scientific consensus terminology for the ligand is used.

In any of the embodiments of this disclosure the checkpoint ligand may be selected from or may exclude any of the following proteins, as well as any active portion, peptide or epitope thereof that elicits signaling in an immunological checkpoint and/or that binds to and elicits signaling within leukocytes or lymphoid tissue-associated cells, such as autoreactive B cells and/or autoreactive T cells: PD-L1 (Sequence 072) and PD-L2 (Sequence 073); CD80 (Sequence 074) and CD86 (Sequence 075); B7RP1 (Sequence 076); B7-H3 (Sequence B7-H3); B7-H4 (Sequence B7-H4); HVEM (Sequence 079); MHC-I (Sequence 080) and MHC-II (Sequence 081) of any allele, CD137L (Sequence 082); OX40 (Sequence 083); CD70 (Sequence 084); GALS (Sequence 085); or any protein, peptide, lipid, glycan, glycolipid, glycoprotein, lipoprotein, nucleic acid, ribonucleoprotein, or deoxyribonucleoprotein that binds to a transmembrane, peripheral membrane, membrane-associated, or cytosolic receptor/protein containing an ITAM, ITIM, or ITSM motif.

In any of the embodiments of this disclosure the immunoregulatory cytokine may be any of the following proteins, as well as any active portion, peptide or epitope thereof that binds to and/or elicits signaling within leukocytes or lymphoid tissue-associated cells, e.g., autoreactive B and/or T cells: Members of the IL-1 family, including IL-1α (Sequence 086), IL-1β (Sequence 087), IL-1Ra (Sequence 088), IL-33 (Sequence 089), IL-18 (Sequence 090), IL-36Ra (Sequence 091), IL-36α (Sequence 092), IL-36β (Sequence 093), IL-36γ (Sequence 094), IL-37 (Sequence 095), and IL-38 (Sequence 096); IL-2 (Sequence 097), IL-3 (Sequence 098), IL-4 (Sequence 099), IL-5 (Sequence 100), IL-6 (Sequence 101), IL-7 (Sequence 102), IL-8 (Sequence 103), IL-9 (Sequence 104), IL-10 (Sequence 105), IL-11 (Sequence 106), IL-12 (Sequence 107), IL-13 (Sequence 108), IL-14 (Sequence 109), IL-15 (Sequence 110), IL-16 (Sequence 111), IL-17 (Sequence 112), IL-19 (Sequence 113), IL-20 (Sequence 114), IL-21 (Sequence 115), IL-22 (Sequence 116), IL-23 (Sequence 117), IL-24 (Sequence 118), IL-25 (Sequence 119), IL-26 (Sequence 120), IL-27 (Sequence 121), IL-28 (Sequence 122), IL-29 (Sequence 123), IL-30 (Sequence 124), IL-31 (Sequence 125), IL-32 (Sequence 126), IL-35 (Sequence 127); an interferon such as a Type I, II, or III interferon; a chemokine of a C, CC, CXC, and CX₃C class; a TNF receptor superfamily ligand, such as OX40L, CD40L, TNF-α, and CD70, and 4-1BBL; or a non-canonical chemokinetic and chemotactic agents, such as Slit1, Slit2, and Slit3; or TGF-β (Sequence 128).

An exemplary PantId can include the checkpoint receptor PD-L1, and the effector, FasL. An exemplary PantId can include the cytokine receptor IL2Rβ, and the effector, IgG1H constant regions 1-3. An exemplary PantId can include the checkpoint receptor CTLA-4, and the effector, IgG1H constant regions 1-3, IgG1H constant regions 2-3, or IgG1H Fc regions.

As used herein and throughout this document, a molecular chimera is any covalently linked or non-covalently associated complex of one or more partners comprised of proteins, domains, peptides, glycans, lipids, nucleic acids, glycoproteins, lipoproteins, ribonucleoproteins, deoxyribonucleoproteins, and covalently-modified peptides.

In one embodiment, this disclosure features methods for the production of PantIds. Such a method may include cloning of (1) a checkpoint receptor, ligand, or immunoregulatory cytokine or any active portion peptide or epitope thereof, as a protein/peptide molecular chimera with (2) an effector, or any active portion thereof that elicits leukocyte, e.g., B cell, apoptosis, necrosis, tolerization, and/or a homodimerization, heterodimerization, trimerization, tetramerization, or oligomerization domain. Cloning and expression can utilize any nucleic acid expression system or combination of expression systems, with or without IRES elements or P2A//T2A picornaviral slip sites or alternative polyprotein/polycistron expression motifs and modalities. Such nucleic acid expression systems can include linear or circular double-stranded or single-stranded RNA or DNA. Such expression systems may include or exclude plasmids containing a bacterial or eukaryotic origin of replication, an antibiotic or affinity selection marker, and/or a prokaryotic or eukaryotic promoter. In one potential embodiment, such a plasmid may include HIV, retroviral, or foamy spumaviral-derived viral sequences including, but not limited to, the viral long-terminal repeat (LTR) and post-transcriptional viral regulatory sequences, includeing the HIV Rev-Response Element (RRE), as well as viral or subviral particles produced therefrom. Alternatively, expression could constitute synthesized peptides and molecular chimeras thereof.

The nucleic acids encoding the PantId may comprise an expression plasmid, a viral vector, a lentiviral vector, or an mRNA. The Pantid may be a synthesized protein, a synthesized peptide, or expressed in transduced or transfected cells comprising the nucleic acids, proteins, or peptides.

Expression systems for the PantId include in vitro systems such as ribosomal translation, or cell based systems such as bacterial culture, archaeal culture, fungal culture, plant culture, or animal cell culture, including CHO cell culture. In addition, in some embodiments, the PantId is expressed in a human cell expression system. In some embodiments, expression of the PantId in a human cell, xenofree expression system reduces the antigenicity of the PantId composition.

In one embodiment, this disclosure features methods of purification of PantId proteins by any column chromatographic, solvent exclusion, precipitation, or magnetic or non-magnetic nano/microparticle methodology, including but not limited to affinity chromatography, high-performance liquid chromatography, size-exclusion chromatography, anion or cation exchange chromatography, reverse-phase chromatography, and immunoaffinity magnetic or non-magnetic particles and beads of any size.

In another embodiment, this disclosure features methods for the introduction of PantIds in cell culture, animal models, and humans as recombinant proteins, including by viral and non-viral protein transduction. Additionally, in this embodiment, the present invention includes methods for therapeutic efficacy or bioactivity assessment and quantification, including, but not limited to, cell viability assays, cell death assays, cell metabolisms assays, cytostatic assays, cell proliferation assays, targeted cell killing assays, immune cell killing assays, flow cytometric assays, Western blot assays, cytokine ELISAs and Western blot assays, whole blood workup assays, leukocyte counts, HPLC and mass spectrometric assays, ELISpot assays, fluorescent and chemiluminescent-linked immunosorbent assays, in vivo imaging, etc.

Another embodiment of this disclosure relates to methods for the discovery, quantification, and characterization of autoimmune B cell responses to checkpoint receptors, their ligands, and immunoregulatory cytokines by reverse-phase protein microarray (RPMA), forward-phase protein microarray, immunosorbent assays (including enzyme-linked, fluorometric, and luminometric), particle-agglutination assays, electrophoretic mobility shift and capillary electrophoresis assays, electrochemical or electroluminescent assays, or single or multiplexed tissue or cell arrays, or flow cytometry.

Also featured in an embodiment of this disclosure are methods for the delivery of PantIds and combinations of PantIds and other therapeutics in animal models of autoimmune disease and cancer.

In one embodiment, this disclosure features the delivery of PantIds and combinations of PantIds and other therapeutics in subjects, including humans or animals, for the treatment of autoimmune diseases or disorders or cancer, whether by intravenous, sublingual, intranasal, intradermal, intramuscular, intraorbital or periorbital, transdermal, or subcutaneous delivery methods.

Compositions may take the form of any standard known dosage form including tablets, pills, capsules, semisolids, powders, sustained release formulation, solutions, suspensions. By way of further example, the compositions may also include preserving agents, solubilising agents, stabilising agents, wetting agents, emulsifying agents,

The therapeutic or pharmaceutical compositions according to the disclosure may comprise a Pantid and a pharmaceutical carrier. The Pantid is preferably essentially pure and desirably essentially homogeneous (i.e. free from contaminating proteins etc). “Essentially pure” protein means a composition comprising at least about 90% by weight of the protein, based on total weight of the composition, preferably at least about 95% by weight. “Essentially homogeneous” protein means a composition comprising at least about 99% by weight of protein, based on total weight of the composition. In certain embodiments, the protein is an antibody. Alternative compositions include lentiviral, retroviral, other viral, and non-viral particles that mediate protein or nucleic acid transduction. In one potential embodiment, “composition” may also include transduced or transfected cells of mammalian or host origin, which produce PantIds after administration.

The amount of Pantid in the formulation is determined taking into account the desired dose volumes, mode(s) of administration etc. The PantId formulation may comprise a pharmaceutically acceptable carrier or diluent. In some aspects, suitable carriers and diluents include buffered, aqueous solutions, isotonic saline solutions, for example phosphate-buffered saline, isotonic water, sterile water, solutions, solvents, dispersion media, delay agents, polymeric and lipidic agents, emulsions and the like. The Pantid may be present in a pH-buffered solution at a pH from about 4-8, and preferably from about 5-7. Exemplary buffers include histidine, phosphate, Tris, citrate, succinate and other organic acids. The buffer concentration can be from about 1 mM to about 20 mM, or from about 3 mM to about 15 mM, depending, for example, on the buffer and the desired isotonicity of the formulation. By way of further example, suitable liquid carriers, especially for injectable solutions, include water, aqueous saline solution, aqueous dextrose solution, and the like, with isotonic solutions being preferred for intravenous, intraspinal, and intracisternal administration and vehicles such as liposomes being also especially suitable for administration of agents.

Other pharmaceutically acceptable carriers, excipients or stabilizers such as those described in Remington's Pharmaceutical Sciences 16th edition, Osol, A. Ed. (1980) may be included in the pre-lyophilized formulation (and/or the lyophilized formulation and/or the reconstituted formulation) provided that they do not adversely affect the desired characteristics of the formulation. Acceptable carriers, excipients or stabilizers are nontoxic to recipients at the dosages and concentrations employed and include; additional buffering agents; preservatives; co-solvents; antioxidants including ascorbic acid and methionine; chelating agents such as EDTA; biodegradable polymers such as polyesters; and/or salt-forming counterions such as sodium.

In one embodiment, therapeutic compositions of this disclosure comprise a carrier “Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ polyethylene glycol (PEG), and PLURONICS™.

“Treating” or “treatment” or “amelioration” refers to both therapeutic treatment and prophylactic or preventative measures; wherein the object is to prevent or slow down (lessen) the targeted autoimmune disease or disorder, or cancer. Those in need of treatment include those already with the disorder as well as those prone to have the disorder or those in whom the disorder is to be prevented, such as subjects who have leukocytes, such as autoreactive B cells that respond to a checkpoint receptor, ligand, or immunoregulatory cytokine.

A subject or mammal is successfully “treated” for an infection if, after receiving a therapeutic amount of a Pantid of this disclosure, according to the methods of the present invention, the subject shows observable and/or measurable reduction in or absence of one or more of the following: reduction in the number of autoreactive B cells or one or more autoimmune symptoms or reduction in cancer.

The term “therapeutically effective amount” refers to an amount of a Pantid effective to “treat” a disease or disorder in a subject or mammal.

### EXAMPLES

Those of skill in the art will appreciate that the following examples are non-limiting examples of cloning and expressing a PantId, and that other methods, vectors, and expression systems may also be used in the cloning of the PantIds of this disclosure. One of skill in the art will also appreciate that the methods, vectors, and expression systems may also be used in the cloning of PantIds comprising other components, as described in this disclosure.

### Example 1—PantId Cloning

The checkpoint receptor, ligand, or immunoregulatory cytokine or any extracellular domain, or active portion peptide or epitope thereof (with or without a signal peptide) is reverse translated from the mRNA sequence. For example, PD-L1, corresponding to amino acids 1-239, is reverse translated using the codon adaptation tool available at the www.jcat.de using the Homo sapiens codon usage option. The resultant sequence is copied and pasted into a new SnapGene. dna file for in silico generation of the final PantId sequence.

The signal peptide of PD-L1, corresponding to amino acids 1-18, is removed and replaced with human serum albumin signal peptide (amino acid sequence MKWVTFISLLFLFSSAYS), after reverse translation. This is copied onto the extreme 5′ end of the PD-L1 sequence.

The extracellular domain of an effector is reverse translated and copied onto the 3′end of the checkpoint receptor, ligand, or immunoregulatory cytokine or any active portion peptide or epitope thereof. For example, FasL, corresponding to amino acids 103-281, is reverse translated and copied onto the 3′end of the PD-L1 sequence.

A linker may also be interposed between the two components. For example, a GGGGS linker or other suitably flexible linker may be used. For example, a GGGGS linker is subsequently pasted in between the two features, allowing molecular chimera flexibility in the final protein. Alternatively, multiples of this linker, including (GGGGS)₂, (GGGGS)₃, (GGGGS)₄, (GGGGS)₅, or any peptide containing 50% or greater total glycine, serine, and threonine content of any length greater than or equal to 2 amino acids.

In some embodiments, an affinity peptide may also be included in the molecular chimera, to facilitate purification. For example, a biotin, avidin or streptavidin-binding peptide (SBP, amino acid sequence MDEKTTGWRGGHVVEGLAGELEQLRARLEHHPQGQREP) can be used. For example, (SBP, amino acid sequence MDEKTTGWRGGHVVEGLAGELEQLRARLEHHPQGQREP) is appended to the end of FasL for immunoaffinity purification.

A stop codon (DNA sequence 5′ TGA 3′) is inserted at the end of the molecular chimera sequence, for example at the end of the SBP, to terminate the protein.

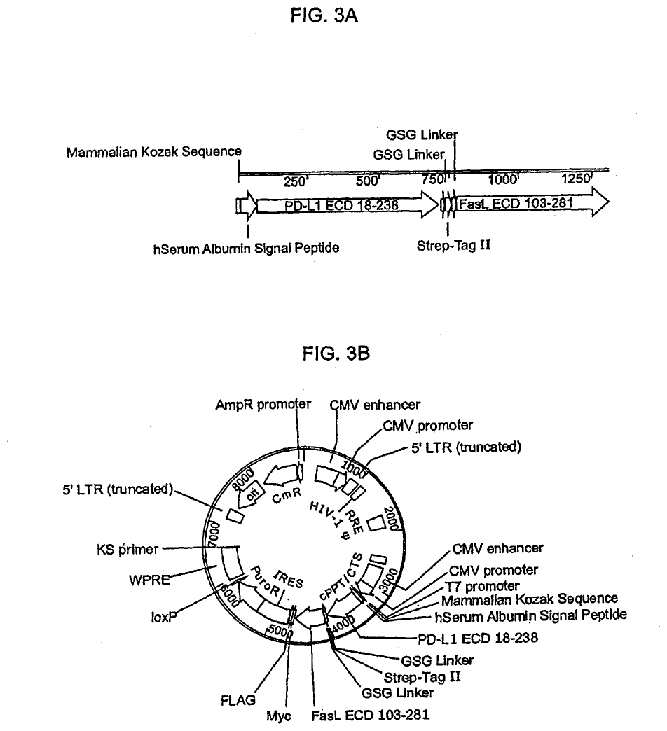

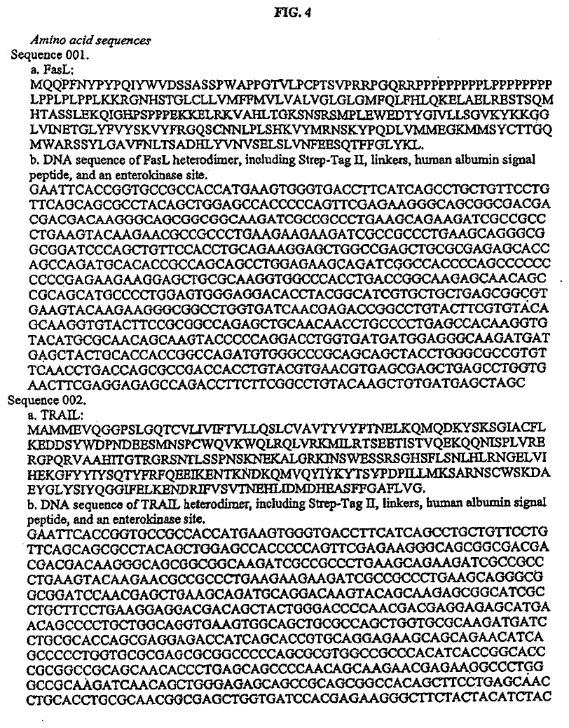

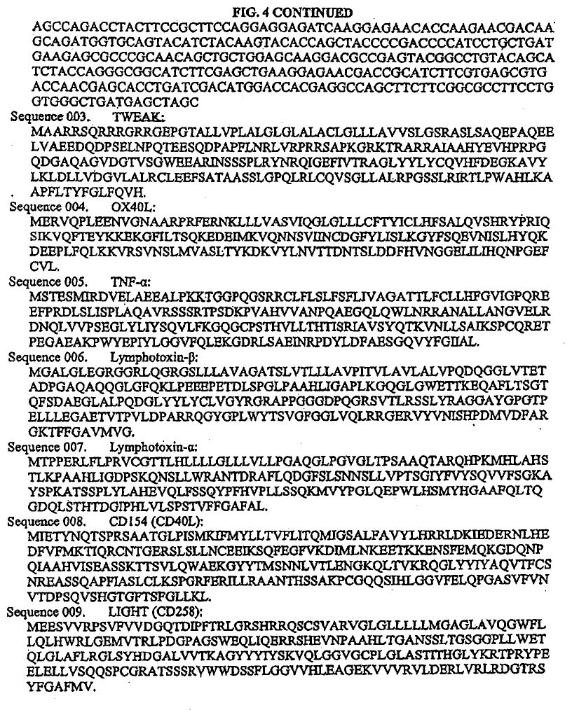

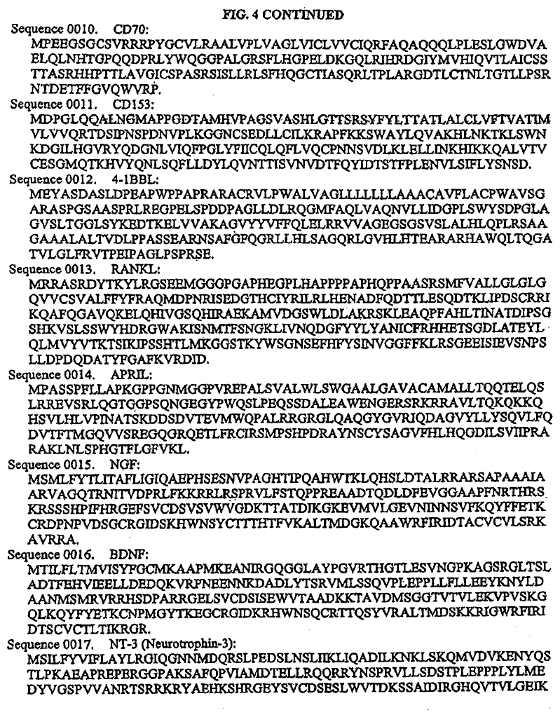

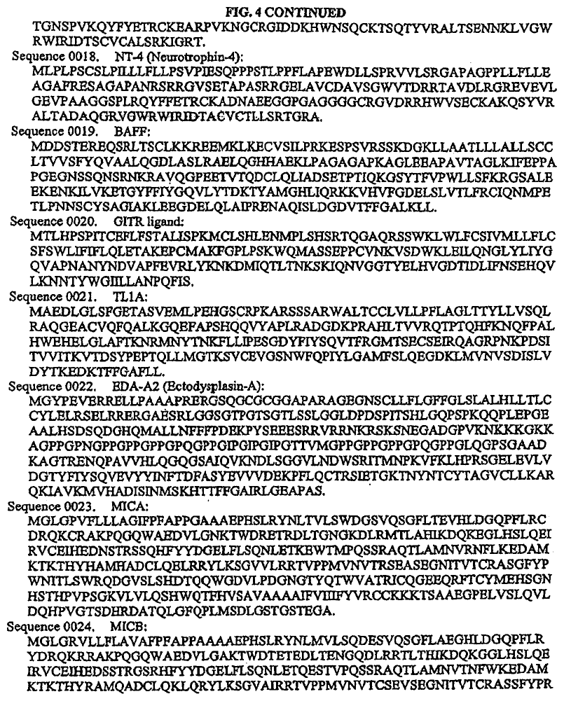

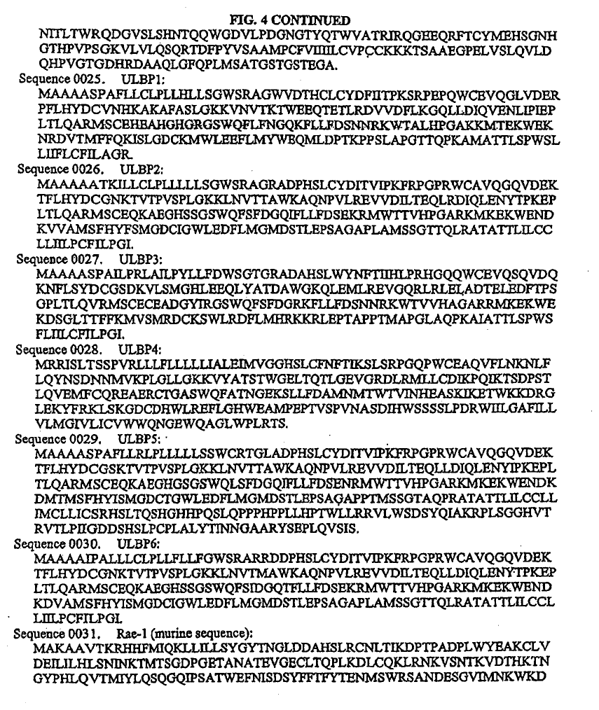

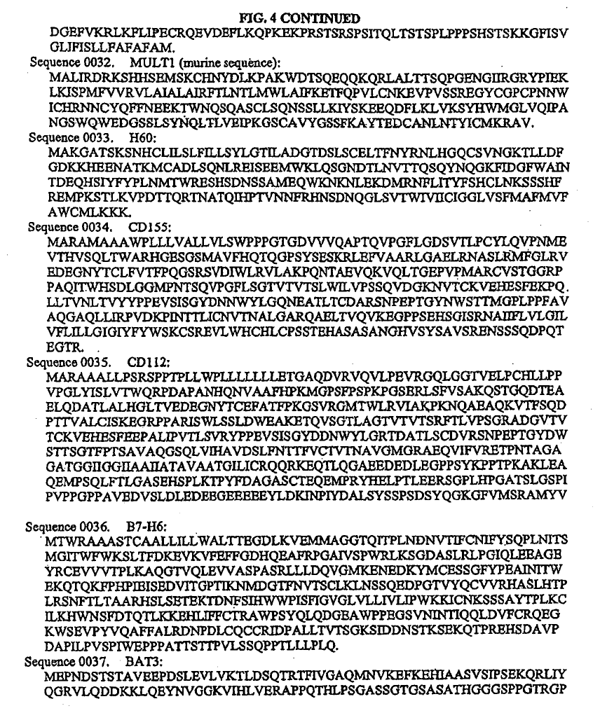

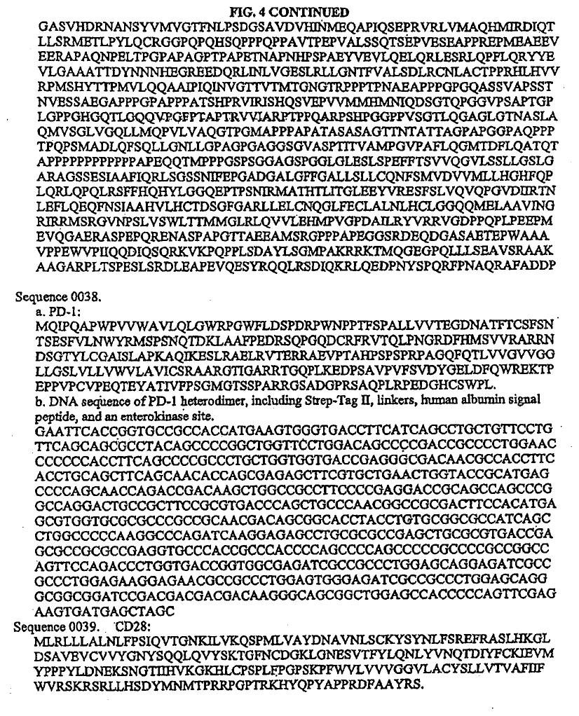

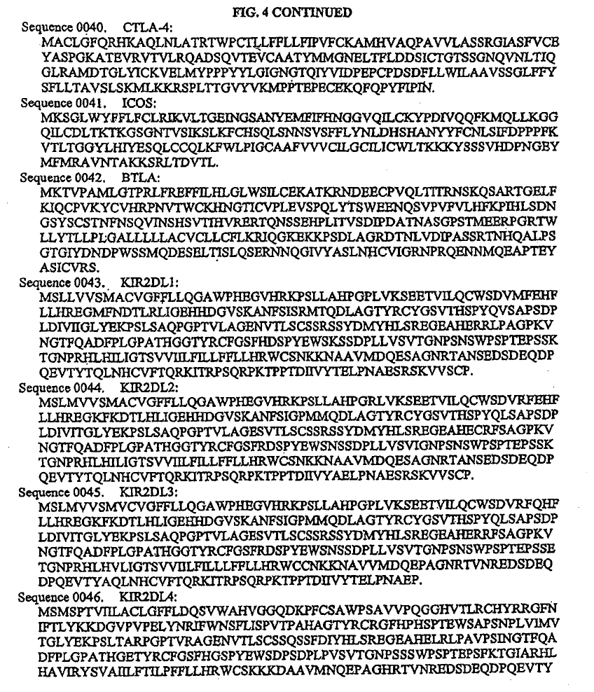

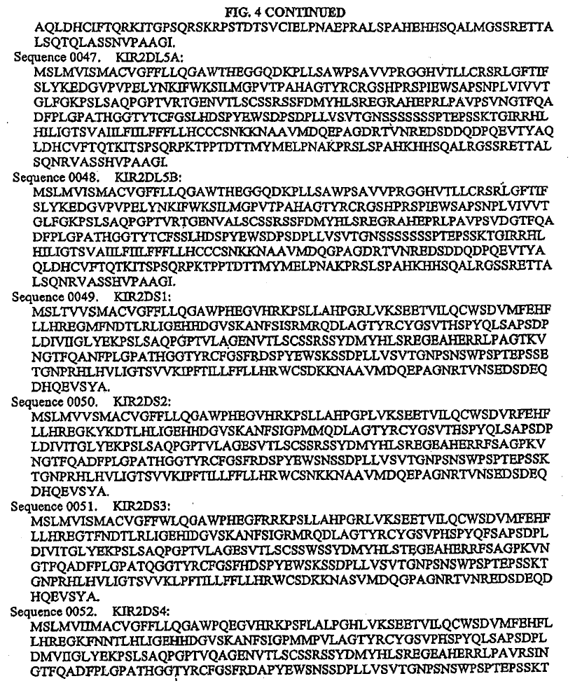

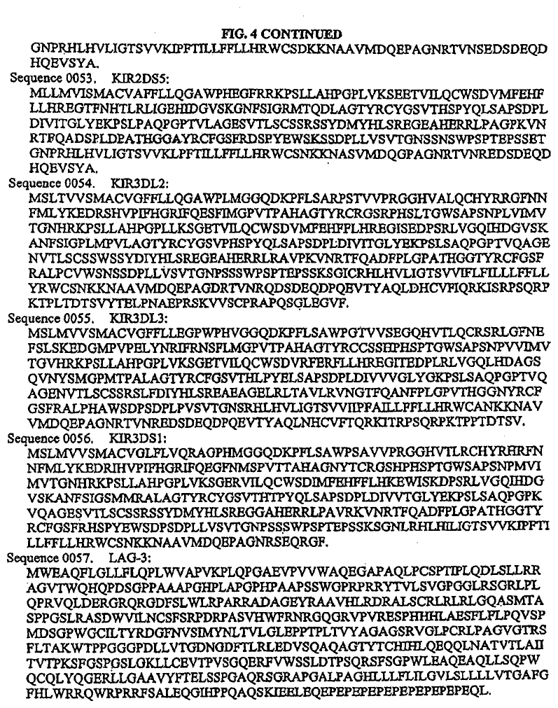

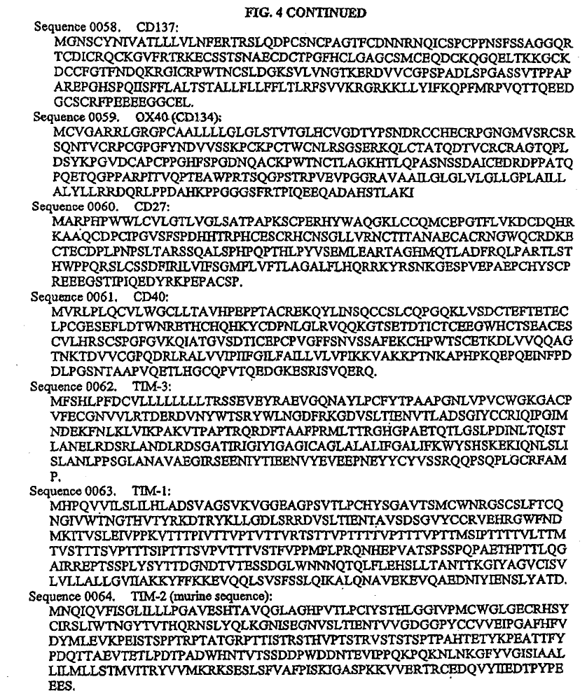

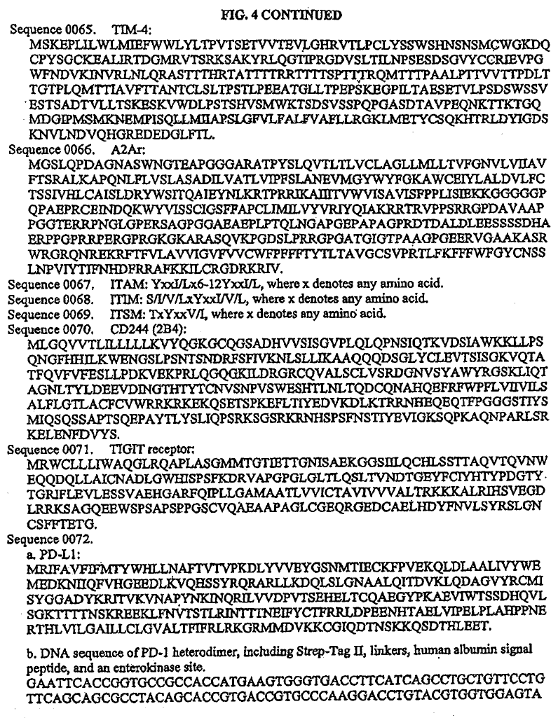

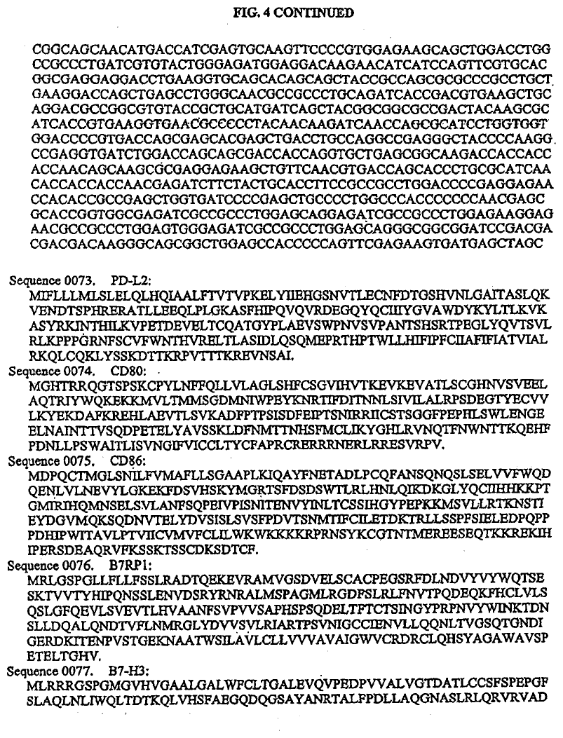

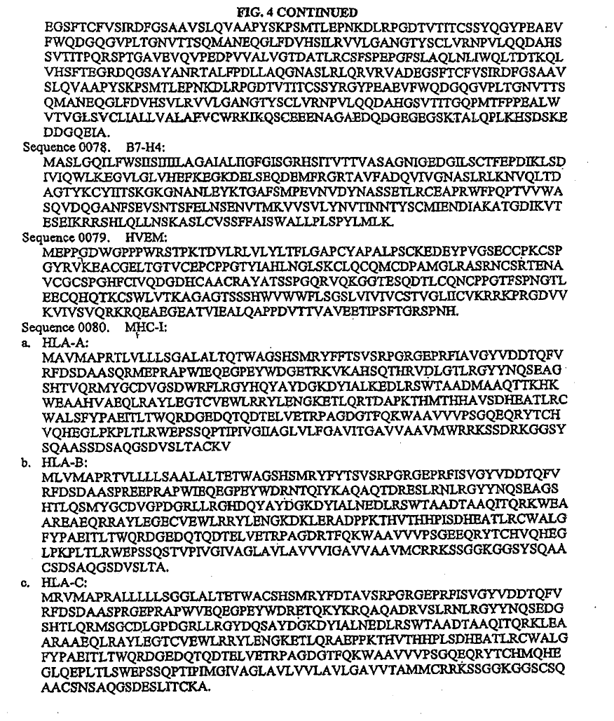

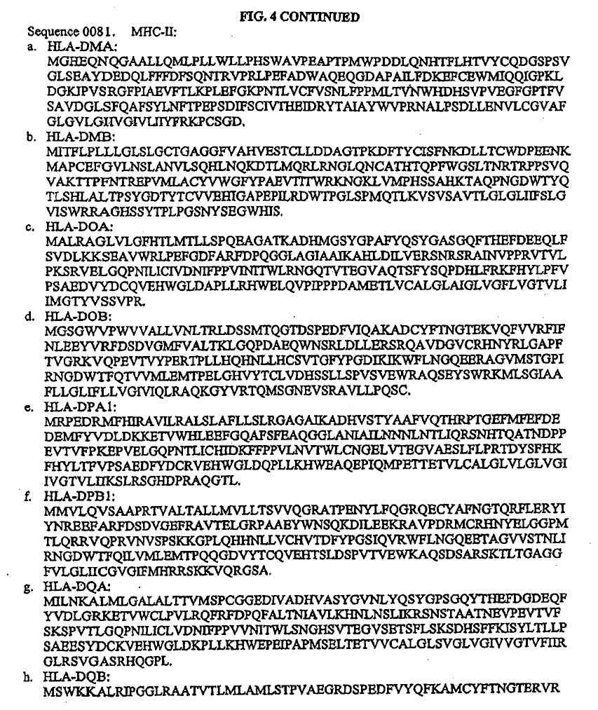

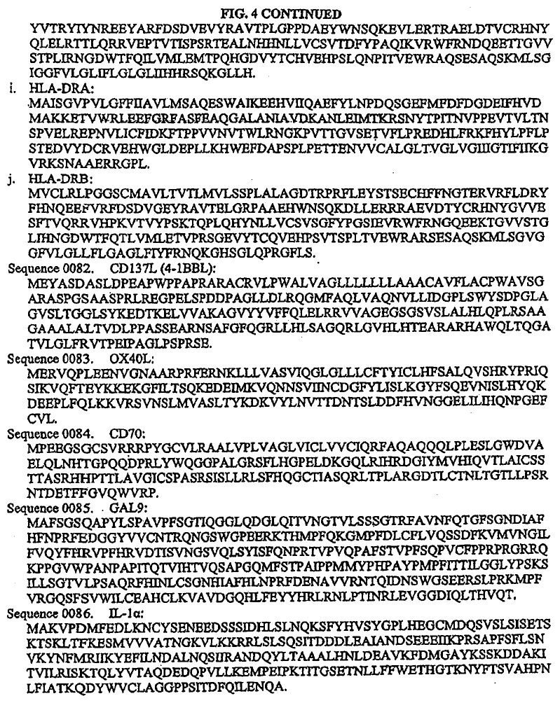

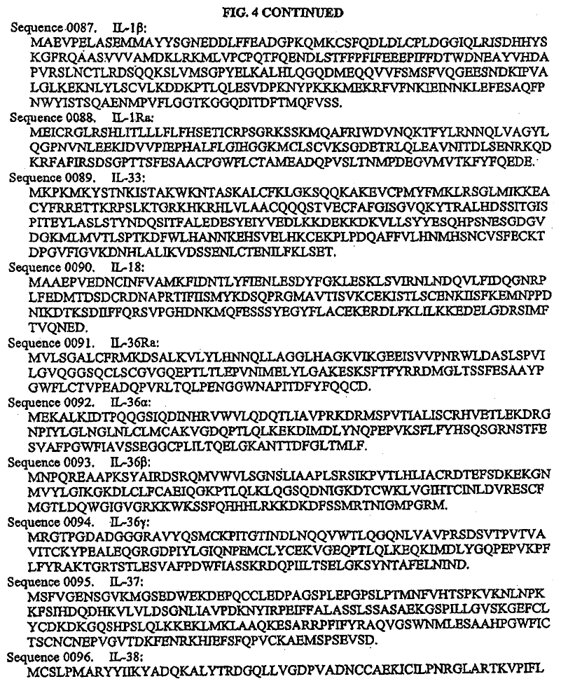

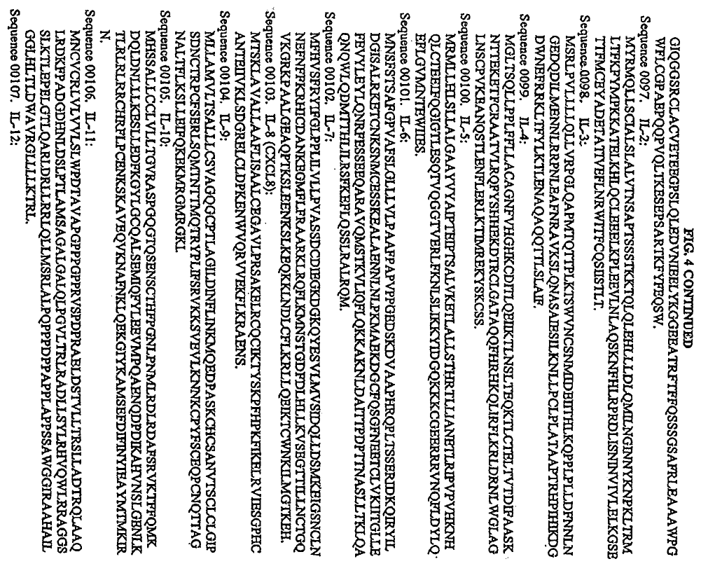

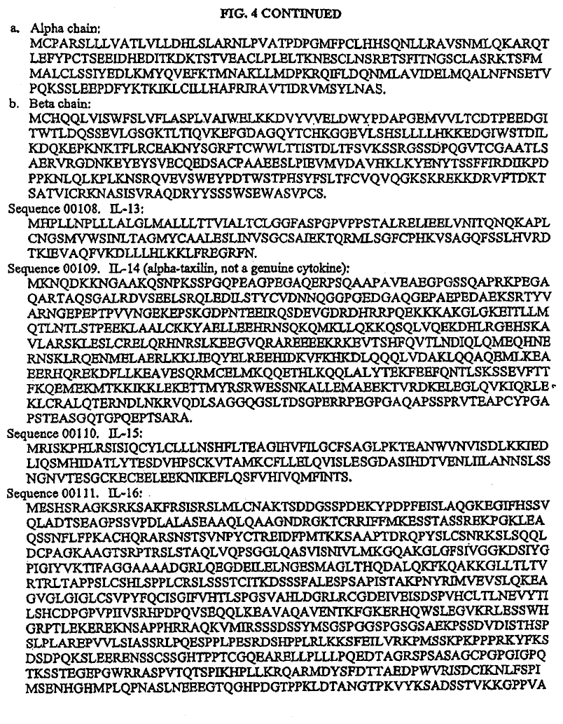

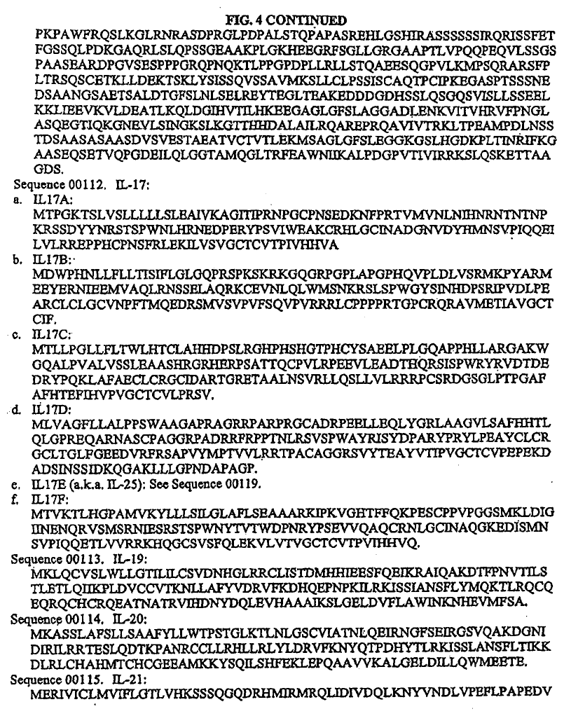

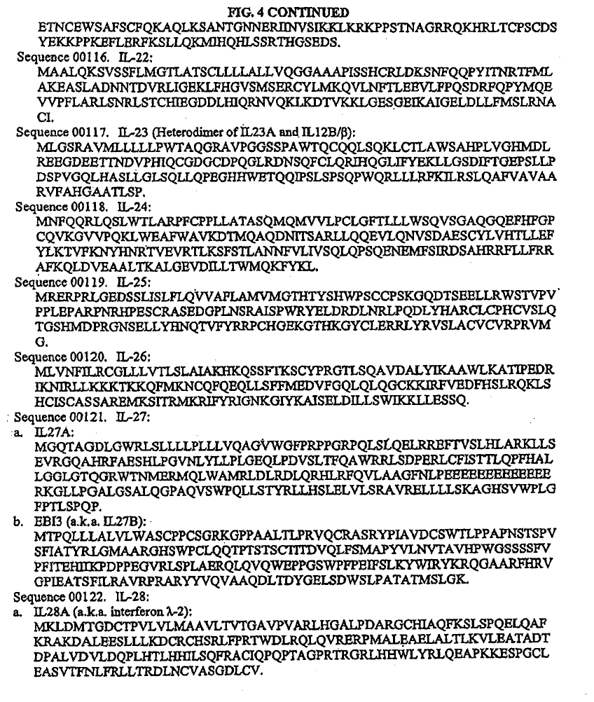

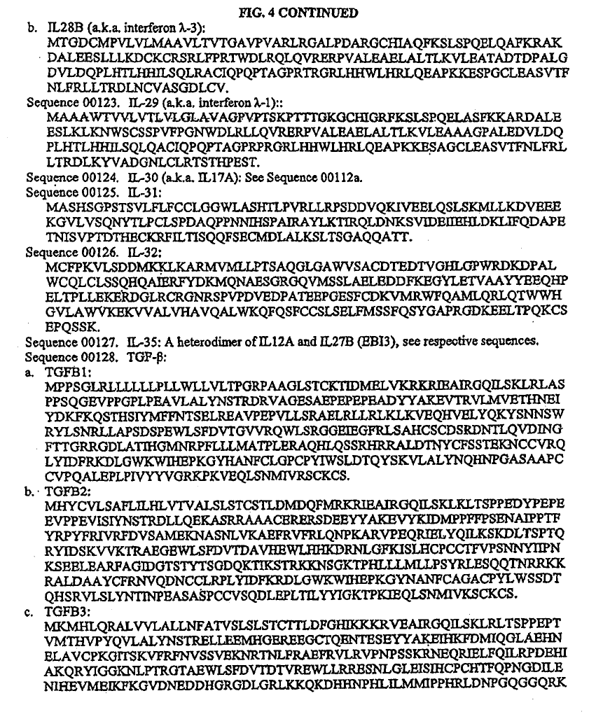

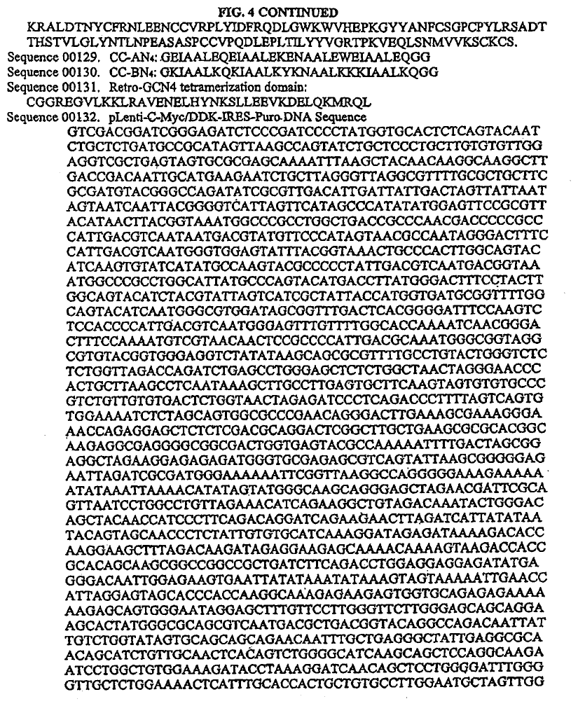

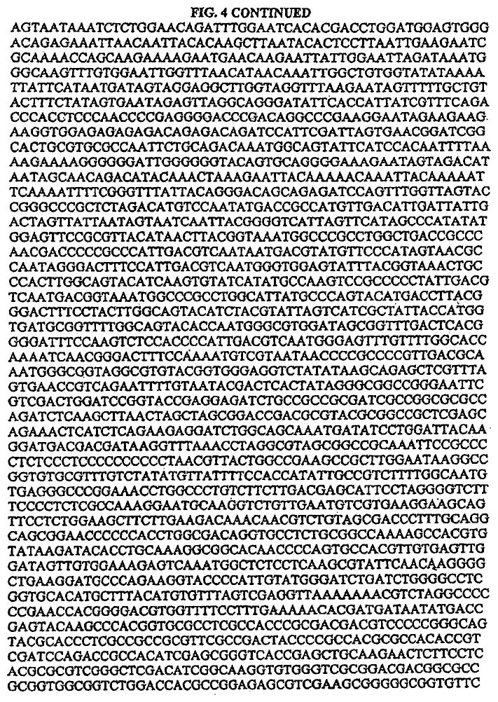

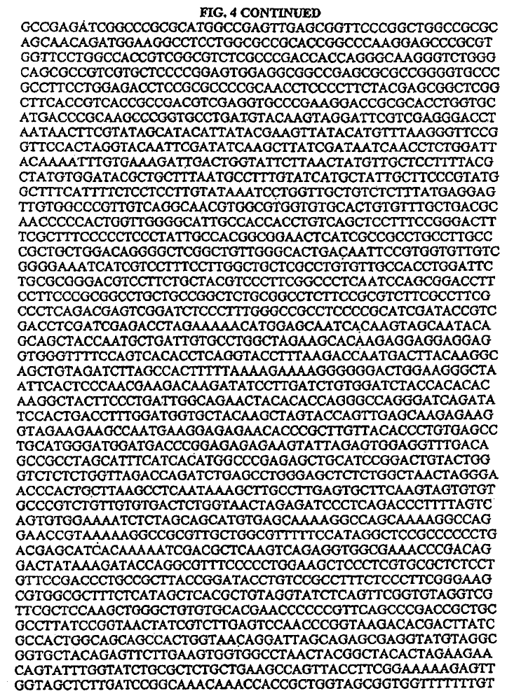

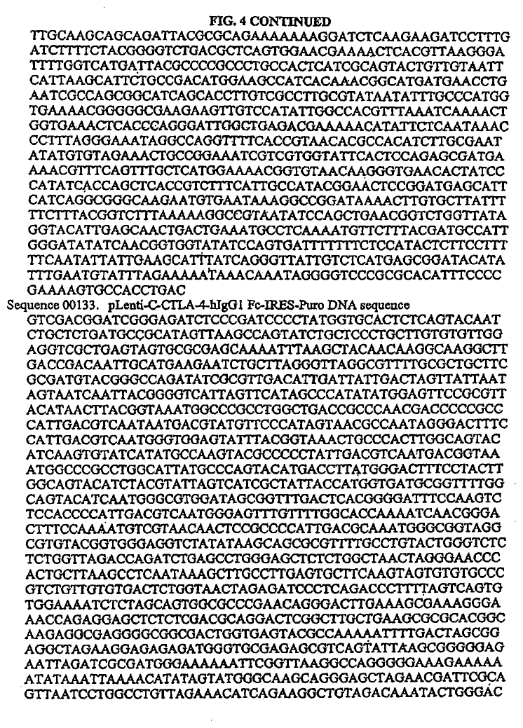

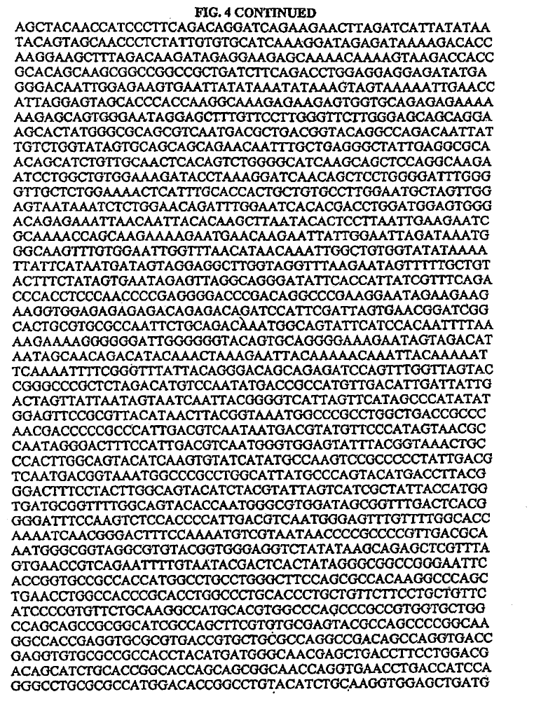

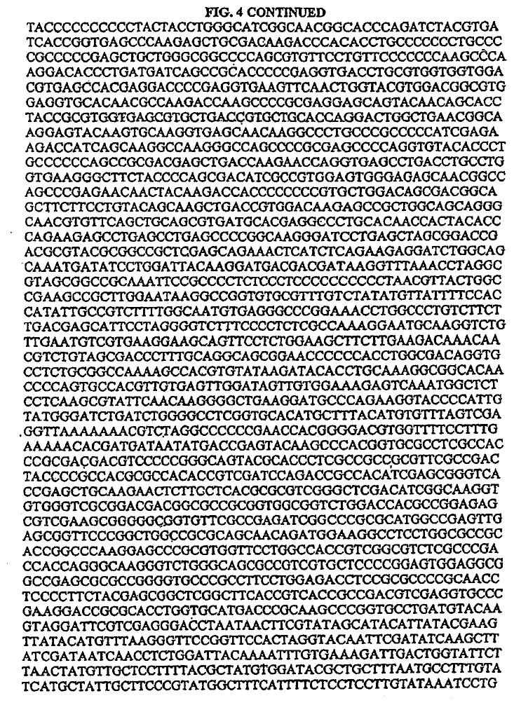

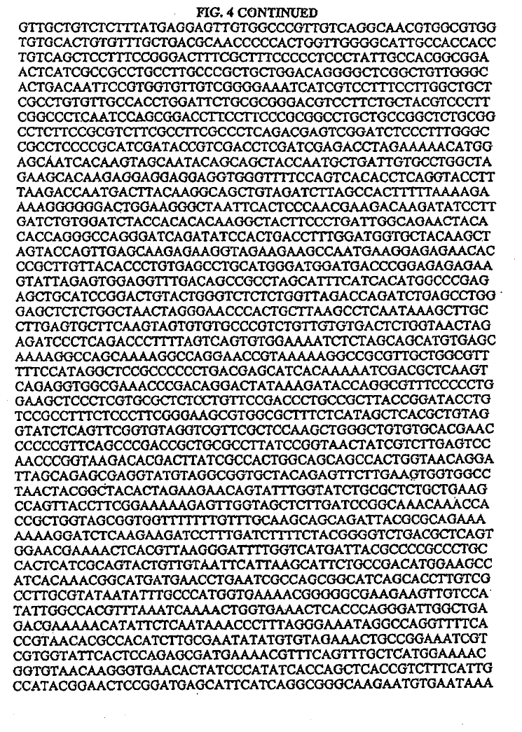

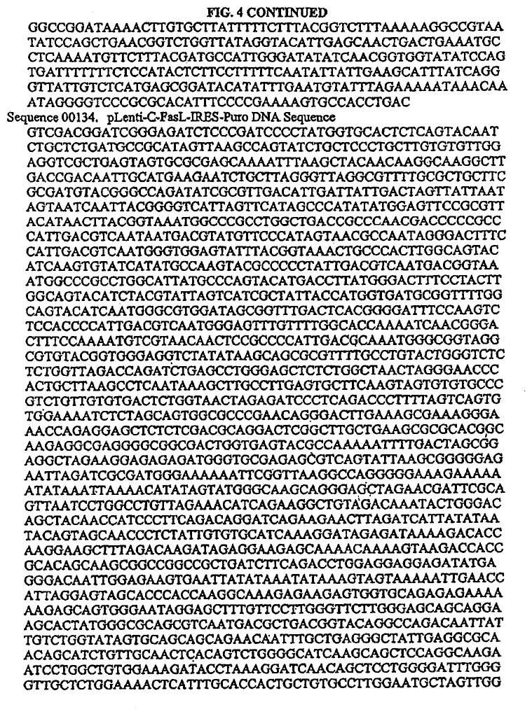

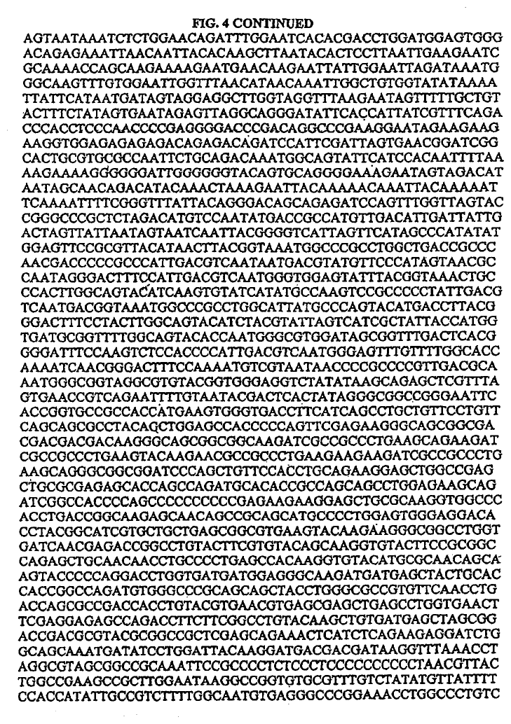

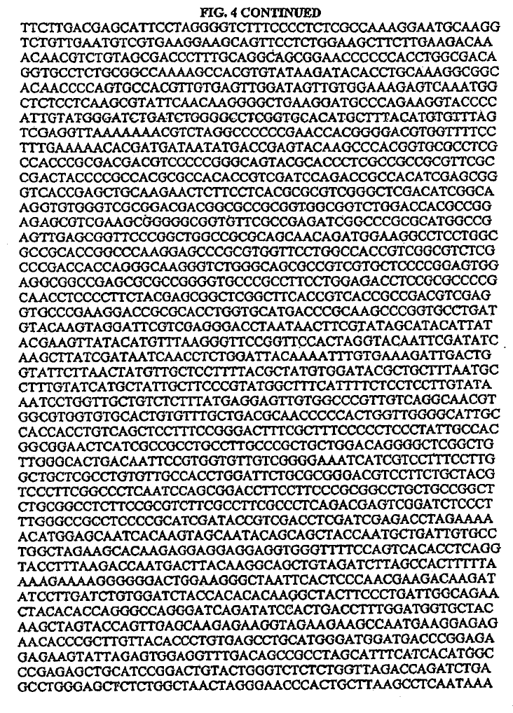

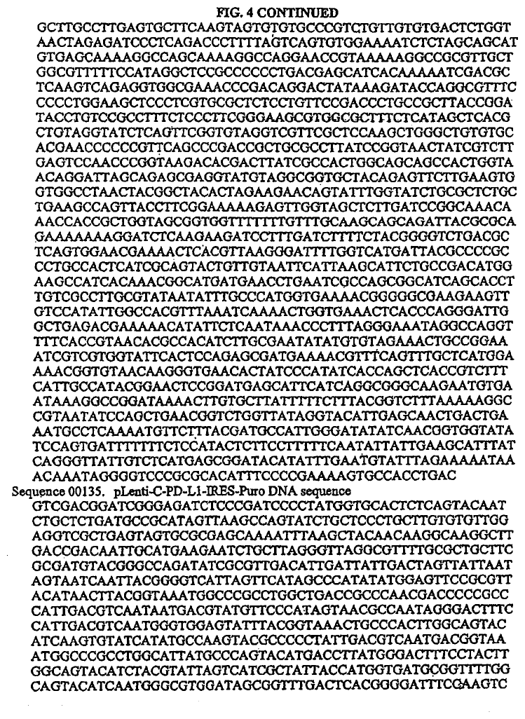

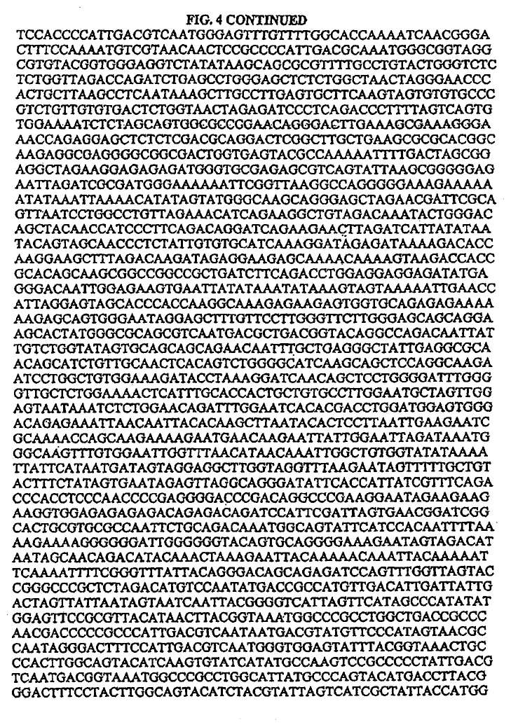

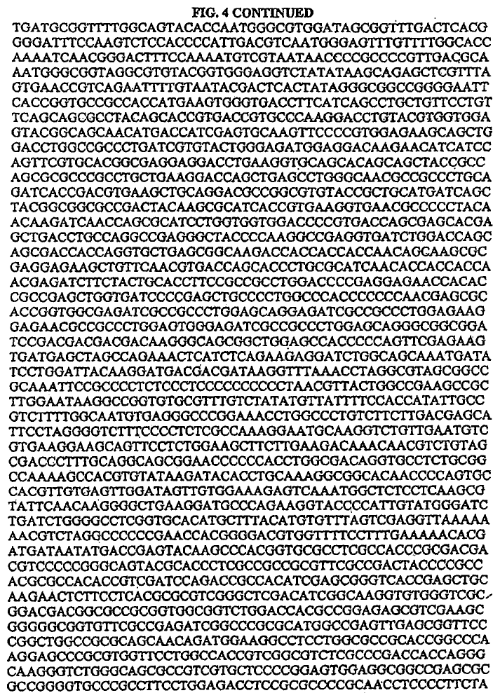

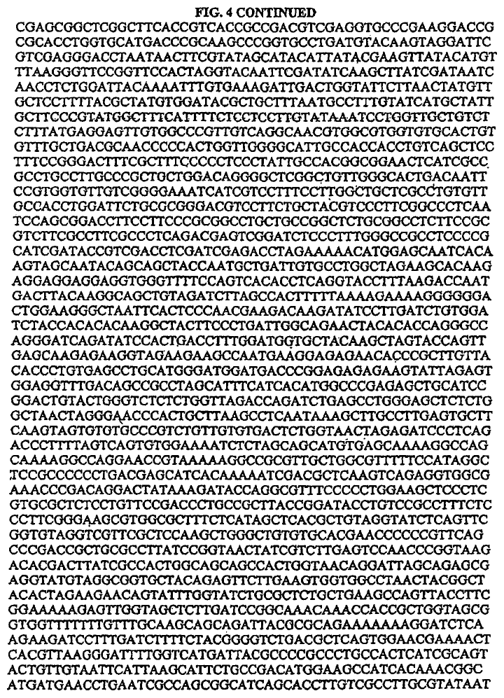

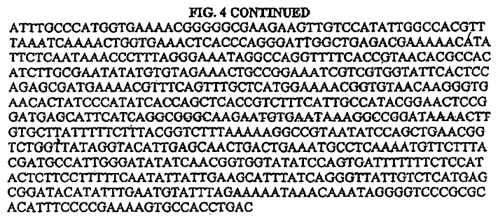

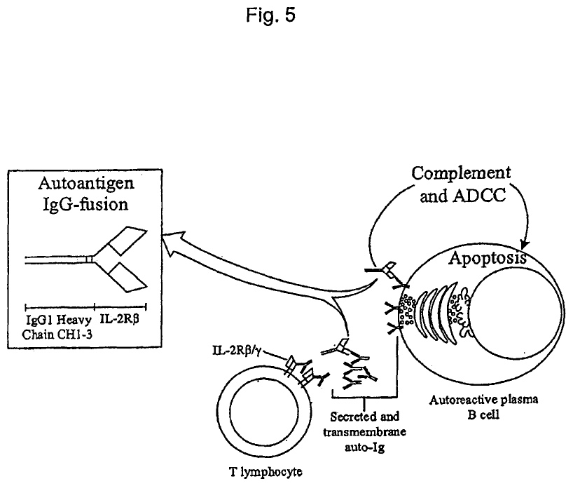

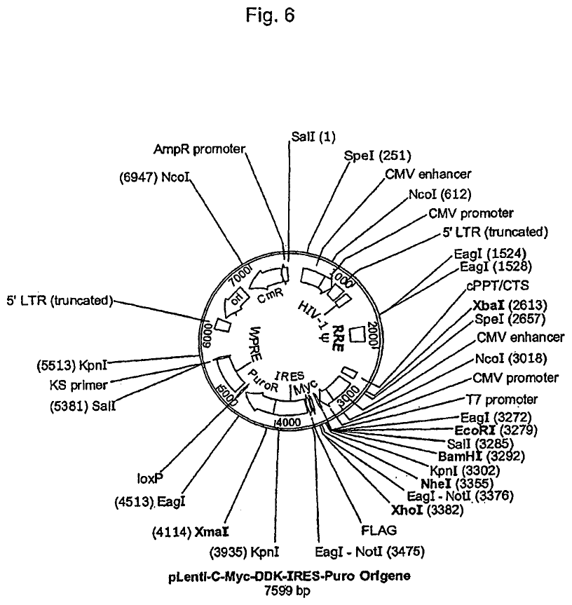

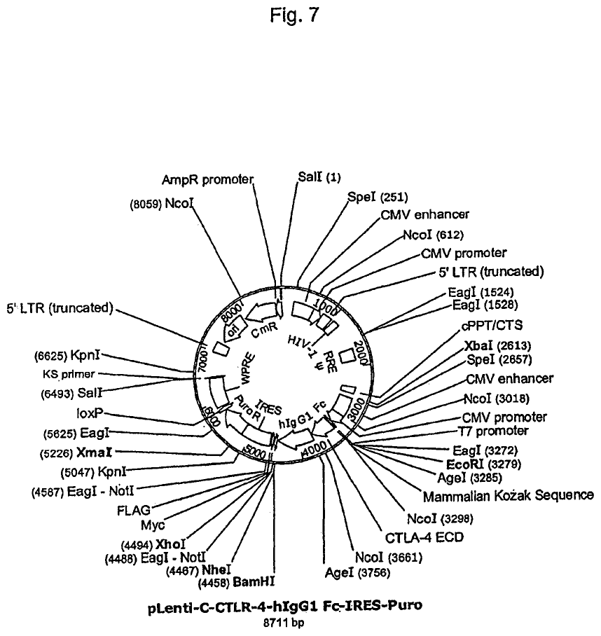

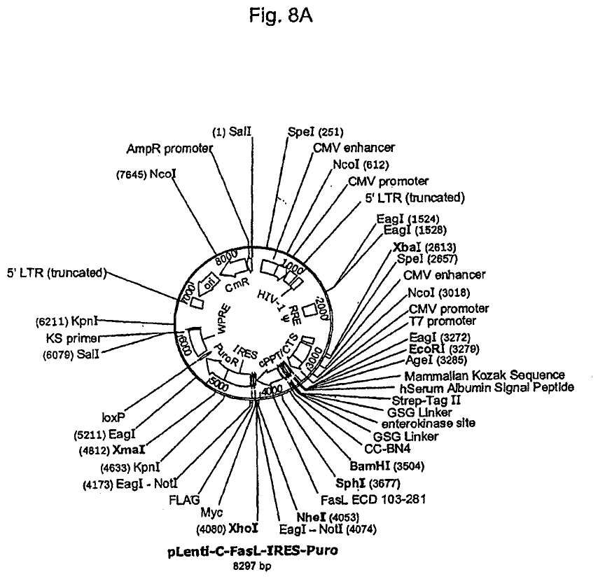

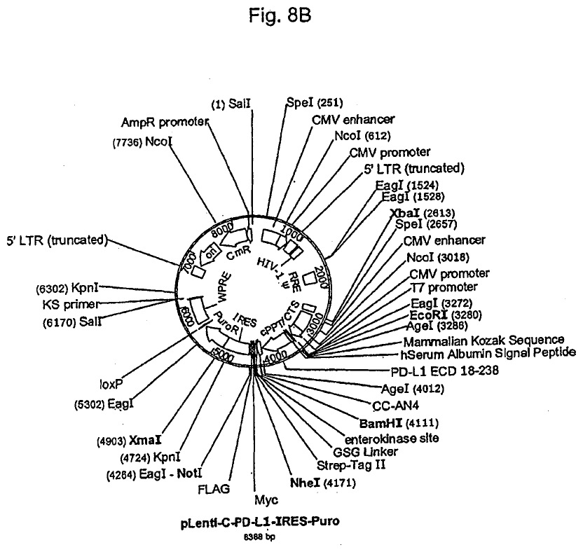

Appropriate restriction enzyme sites may be added to the respective DNA termini for cloning into the expression vector. For example, a 5′ terminal EcoRI site (5′ GAATTC 3′) and a 3′ BamHI site (5′ GGATCC 3′) are copied onto the respective DNA termini for cloning into a suitable vector, such as pLenti-C-Myc-DDK-IRES-Puro. The final in silico-generated map is shown in FIG. 3A.

This sequence is exported as text for gene synthesis by GENEWIZ as purified plasmid cloned into pUC57-Amp.

The pUC57-Amp is transformed into DH5a chemically competent bacterial cells, which, after screening, results in a single clone.

This clone is cultured in LB broth with 100 μg/ml ampicillin, and plasmid is extracted using a QIAGEN plasmid extraction miniprep kit.

This DNA is digested with EcoRI-HF and BamHI, from New England Biolabs, to liberate the PD-L1-FasL fragment, which is isolated by agarose gel electrophoresis and extraction.

This fragment is admixed at a 3:1 molar ratio with SAP-dephosphorylated, BamHI-HF/EcoRI-HF double-digested, and PCR column-purified pLenti-C-Myc-DDK-IRES-Puro linearized DNA.

Fragments are ligated using 1-100U of T4 DNA ligase, from New England Biolabs.

The resulting DNA is transformed into DH5a, and clones are screened by BamHI-HF/EcoRI-HF double digestion for the presence of the insert. A single insert-positive clone is chosen for subsequent transfection, characterization, and purification of recombinant PantId. An example of the positive clone plasmid map is shown in FIG. 3B.

### Example 2—Transfection of PantId Expression Vector

HEK293T cells are thawed in cryomedium, consisting of 7% DMSO in FBS, at 37° C. for 3 minutes.

The cell suspension is diluted with an additional 5 ml of DMEM with 10% FBS, mixed by inverting the tube, and then centrifuged at 300×g for 5 minutes at room temperature.

The supernatant is decanted, and cells are resuspended into 15 ml of DMEM with 10% FBS.

Cells are cultured in a T-75 flask for 1-3 days, until they achieve greater than 70% confluency.

At this point, the cell culture medium is removed, and cells are trypsinized with 3 ml 0.25% trypsin-EDTA for 5 minutes at 37° C.

Cells are triturated by pipetman vigorously for 30 seconds prior to dilution in 7 ml of DMEM with 10% FBS.

Cells are counted, and 3·10⁶ cells are pipetted into a 10 cm Petri dish in a total volume of 10 ml of DMEM with 10% FBS with pen/strep. Cells are cultured for an additional 12-18 hours prior to transfection.

The following day, the cell culture supernatant is replaced with 7 ml serum-free DMEM.

For lentiviral particle production, 10 μg of pLenti-C-PD-L1-FasL-IRES-Puro is admixed with 7.5 μg of pCMVA8.2 and 2.5 μg pHCMV-G and 1.5 ml serum-free DMEM. For protein expression, 20 μg of pLenti-C-PD-L1-FasL-IRES-Puro is mixed with 1.5 ml of serum-free DMEM.

Alongside this, 60 μl of Lipofectamine-2000 reagent (Life Technologies) is mixed with 1.5 ml of serum-free DMEM.

Both mixtures are allowed to incubated for 5 minutes at room temperature.

After this, the DNA and Lipofectamine solutions are mixed and incubated for 20 minutes at room temperature.

The liposomal-DNA mixture is applied dropwise to cells in the 10 cm Petri dish.

The cells are transfected at 37° C. and 5% CO₂ for 4-6 hours, prior to removal the transfection supernatant and replacement with 10 ml DMEM with 10% FBS and pen/strep.

Cells are cultured for an additional 48 hours prior to harvesting protein or lentiviral particles.

For lentiviral particles, the supernatant is aliquoted as 0.5 or 1 ml aliquots and stored at −80° C.

For protein production, the supernatant is harvested and admixed with protease inhibitor cocktail prior to storage at −80° C.

### Example 3—PantId Immunoaffinity Purification

0.1 mg of streptavidin magnetic beads (Life Technologies) are washed 3 times with 2 ml PBS with 0.1% BSA using a magnetic particle concentrator (MPC).

PantId-containing supernatant is mixed with 1 mg of washed streptavidin beads.

The sample with beads is mixed by end-over-end rocking for 30 minutes at room temperature.

The beads are concentrated on a magnetic particle concentrator (MPC) for 1 minute prior to washing 3 times with PBS with 0.1% BSA.

The sample is eluted in 0.5 ml PBS with 1-10 mM biotin, after incubating for 10 minutes with gentle shaking.

The streptavidin-magnetic beads are removed by MPC, allowing collection of the eluted protein.

The PD-L1-FasL PantId is desalted using Zeba spin desalting columns (Life Technologies) to remove residual biotin.

Protein concentration is estimated by BCA protein assay prior to storage.

For long-term storage, PantId is diluted 50% in glycerol prior to storage at −80° C. Alternatively, the PantId is aliquoted into 50 μl aliquots prior to storage at −20° C.

### Example 4—In Vitro Characterization of PantId-Mediated Cell Killing

50 ml of patient peripheral blood is diluted 2-fold in 1×DPBS before overlay on an equal volume of Ficoll lymphocyte separation medium.

Centrifuge at 400×g for 30-45 minutes, and then aspirate the upper layer.

The peripheral blood mononuclear cell (PBMC) layer is aspirated and transfered to a new 50 ml conical tube.

Wash 3 times with 50 ml 1×DPBS by centrifugation at 300×g for 5 minutes.

Resuspend the cells to 1.10⁵ cells per ml in RPMI+10% FBS and then plate them into a 96-well plate using 100-200 μl per well.

Culture the cells overnight at 37° C. and 5% CO₂.

The following day, assign columns to different treatment groups with column 1 being an untreated control, columns 2-4 being treated with 1.5-100 μg/ml PantId as serial 2-fold dilutions in triplicate, and column 5 being a positive control for cytotoxicity and containing 0.1% Triton X-100. Columns 6-10 are similarly treated for simultaneous B and T cell staining. Columns 11 and 12 are reserved for isotype controls and single-stain controls.

Incubate for 6 hours at 37° C. and 5% CO₂.

The supernatant is removed and replaced with 50-100 μl of trypsin at 37° C. for 5 minutes.

Add 150 μl of RPMI with 10% FBS, and then wash twice with FACS buffer (PBS with 0.5% BSA and 0.1% sodium azide).

The cells are resuspended with 200 μl FACS buffer, and then add 5 μl of 7-AAD per well, 5 μl of AlexaFluor 488-conjugated anti-human CD19 (BioLegend) to stain for B cells, or 5 μl of AlexaFluor 488-conjugated anti-human CD3 (BioLegend) to stain for T cells, or 5 μl of the appropriate isotype control (BioLegend).

The cells are washed twice with FACS buffer to remove residual antibody and 7-AAD.

The cells are resuspended in 200 μl of FACS buffer and flow cytometric analysis is performed. Dead cells appear as 7-AAD-positive events, and the relative distribution of these events among CD19-positive, CD3-positive, and CD19 or CD3-negative populations can be used to preliminarily assess specificity.

### Example 5—Screening of Patient Serum

In some embodiments a protein array is used to screen patient serum. In some embodiments, the array may be, for example, a ProtoArray® Human Protein Microarray. The array may also comprise a plurality of selected checkpoint receptors, ligands, or immunoregulatory cytokines or any extracellular domain, or active portion peptide or epitope thereof.

As an example, when a ProtoArray® Human Protein Microarray is used, immediately place the mailer containing the ProtoArray® Human Protein Microarray at 4° C. upon removal from storage at −20° C., and equilibrate the mailer at 4° C. for at least 15 minutes prior to use.

Place ProtoArray® Human Protein Microarrays with the barcode facing up in the bottom of a 4-chamber incubation tray such that the barcode end of the microarray is near the tray end containing an indented numeral. The indent in the tray bottom is used as the site for buffer removal.

Using a sterile pipette, add 5 mL Blocking Buffer into each chamber. Avoid pipetting buffer directly onto the array surface.

Incubate the tray for 1 hour at 4° C. on a shaker set at 50 rpm (circular shaking). Use a shaker that keeps the arrays in one plane during rotation. Rocking shakers are not to be used because of increased risk of cross-well contamination.

After incubation, aspirate Blocking Buffer by vacuum or with a pipette. Position the tip of the aspirator or pipette into the indented numeral and aspirate the buffer from each well. Tilt the tray so that any remaining buffer accumulates at the end of the tray with the indented numeral. Aspirate the accumulated buffer. Important: Do not position the tip or aspirate from the microarray surface as this can cause scratches. Immediately proceed to adding the next solution to prevent any part of the array surface from drying which may produce high or uneven background.

### Probe the Array:

Use forceps to remove the slide from the 4-well tray. Insert the tip of the forceps into the indented numeral and gently pry the edges of the slide upward. Pick up array with a gloved hand taking care to only touch the array by its edges. Gently dry the back and sides of the array on a paper towel to remove excess buffer. Note: To ensure that the array surface remains wet, do not dry more than 2 arrays at a time before adding the diluted probe, which may, in some instances comprise a labeled anti-human antibody, e.g., a fluorescent or chemiluminescent labeled anti-human antibody, and LifterSlip™ coverslip.

Dilute the serum I:1000 into washing buffer and then place 5 ml of diluted serum in washing buffer into the appropriate chambers of the container.

Incubate for 90 minutes at 4° C. keeping the 4-well tray flat with the array facing up (no shaking).

Add 5 mL cold Washing Buffer.

Wash 5 minutes with gentle agitation at 4° C.

Remove Washing Buffer by aspiration.

Repeat wash steps 4 more times.

Add 5 mL of secondary antibody diluted in Washing Buffer to the indentation at the numbered end of the incubation tray and allow the liquid to flow across the slide surface. To avoid local variations in fluorescence intensity and background, avoid direct contact with the array. Do not pour the antibody solution directly on the slide.

Incubate for 90 minutes at 4° C. with gentle circular shaking (˜50 rpm), unlike the primary stain.

Remove secondary antibody by aspiration.

Wash with 5 mL fresh Washing Buffer for 5 minutes with gentle agitation at 4° C. Remove Washing Buffer by aspiration.

Repeat wash step 4 more times.

### Drying the Array:

Use forceps to remove the array from the 4-well tray. Insert the tip of the forceps into the indented numeral and gently pry the edges of the slide upward (see figure below). Pick up the slide with a gloved hand taking care to touch the slide only by its edges. Tap the slide on its side to remove excess fluid but avoid drying of the array. Place on a flat surface or benchtop.

Place the array in a slide holder (or a sterile 50-mL conical tube). Ensure the slide is properly placed and secure in the holder to prevent damage to the array during centrifugation. Briefly dip the slide holder containing the arrays into room temperature distilled water one time to remove salts. If you are not using a slide holder, dip the array into a 50-mL conical tube filled with room temperature distilled water one time.

Centrifuge the array in the slide holder or 50-mL conical tube at 200×g for 1 minute in a centrifuge (equipped with a plate rotor, if you are using the slide holder) at room temperature. Verify the array is completely dry. After slides have been probed and dried, they can be stored either vertically or horizontally. 4. After drying, store the arrays vertically or horizontally in a slide box protected from light. Avoid prolonged exposure to light as it will diminish signal intensities. To obtain the best results, scan the array within 24 hours of probing.

Insert array into the fluorescence microarray scanner.

### Scanning the Array:

Adjust scanner settings.

Preview the microarray and adjust settings, if needed.

Scan the microarray.

Save image data.

Export and analyze results

### Analyzing the Array:

Perform a Student's t-test on the array duplicates between the control serum and autoimmune patient serum to identify samples with a P-value of 0.05 or less.

From this subset, exclude those antigens that are above a below a cutoff threshold for the ratio of the autoimmune patient serum fluorescent intensity over the control patient serum fluorescent intensity: this is to exclude high-significance, low fold-change hits in the array.

Exclude samples that are below 3-fold, 5-fold, or 10-fold above the local array background to exclude autoantigens that are only marginally above the background.

Annotate the autoantigens by looking up their associated RefSeq ID using PubMed databases.

### Example 6—Mouse Model Demonstration of Efficacy

In some embodiments, animal models, such as a mouse model may be used to demonstrate the efficacy of the PantIds of this disclosure. As a non-limiting example of an efficacy model, a vector, e.g., a lentiviral vector, e.g., pLenti-C-Myc/DDK-IRES-Puro is modified to include a doxycycline-inducible Cre recombinase and a second transcriptional unit, containing a nucleic acid encoding a PantId molecular chimera of this invention, such as CD22 promoter-5′UTR-LoxP₁-PolyA Signal₁-LoxP₂-PD-1-IgG Fc-3′ UTR-PolyA Signal₂. Introduction of doxycycline into mouse water or food, or by injection, causes expression of Cre recombinase. In the absence of Cre, the CD22 promoter drives the expression of an empty mRNA due to an early PolyA signal, which terminates transcription before the molecular chimera, e.g., the PD-IgG Fc, in this non-limiting example. In the presence of Cre, recombination between the LoxP sites results in removal of the first polyA signal and allowing for PD-1-IgG Fc molecular chimera. Thereafter, PD-1-IgG Fc binds to PD-L1 and PD-L2 on cells, antagonizing the tolerogenic effects of these ligands: additionally, the PD-1-IgG Fc binds to PD-1-expressing Tregs cells, and targets them for cell killing, thus eliminating another tolerogenic mechanism. Moreover, the CD22 promoter drives B cell-specific expression. Resultantly, an autoimmune disease that is perfectly mimetic of autoreactive B-cell mediated checkpoint receptor disinhibition is produced. This model will allow for the testing of PantIds in a physiologically relevant system with clear endpoints—the amelioration of the induced autoimmune disease. The methods described below are useful for demonstrating the efficacy of any PantIds that target autoreactive B cells through their B cell receptor (BCR), resulting in clonal deletion. Clonal deletion of anti-checkpoint protein autoreactive B cells will result in significant mitigation of autoimmune-associated inflammation, morbidity, and mortality.

Lentiviral particles are produced as described above by co-transfection with helper plasmids into HEK293T cells.

Mouse BALB/C blastocysts are purchased from Jackson Laboratory and cultured on feeder cells using stem cell culture medium.

Blastocystes are transduced in 6-well plates with an MOT of 1.

After 24 hours, the medium is replaced.

After 48 hours, blastocysts are selected using 1 μg/ml puromycin.

After an additional 48 hours, the blastocysts are washed twice with PBS and then resuspended.

Blastocysts are then transferred into pseudopregant BALB/c uteri by transfer pipette¹³.

After birth, pups are genotyped and inbred to generate a homozygous F2 generation for the study.

These mice are split into 5 groups of 5 mice. Group 1 will receive doxycycline with no treatment, group 2 will receive no doxycycline, group 3 will receive doxycycline and 100 μg/kg PD-L1-FasL PantId twice weekly, group 4 will receive doxycycline and 500 μg/kg PD-L1-FasL PantId twice weekly, and group 5 will receive doxycycline and 1 mg/kg PD-L1-FasL PantId twice weekly. After 2 weeks of autoimmunity induction with doxycycline, PantIds will be administered by intravenous injection. After 3 weeks of treatment by intravenous tail vein injection, mouse tail vein blood will be harvested for IL-2, IL-4, IL-17, TGF-β, and IFN-γ ELISA. Additionally, immune-related symptoms will be scored on a 1-5 scale, which will be monitored weekly after 1 week of PantId treatment. After the end of the study, endpoints will be analyzed to determine PantId therapeutic efficacy relative to the non-autoimmune control.

The method described in this example can be carried out using any of the PantIds disclosed herein.

### Example 7—Cloning of an Exemplary PantId Comprising an Autoantigen-Fc

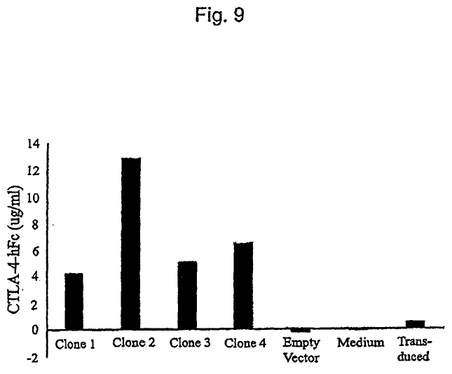

A CTLA-4-Fc PantId was produced in HEK293T cells by expressing an exemplary CTLA-4-hFc construct in a lentiviral expression vector. The PantId comprised CTLA-4 fused to a hIgG₁ Fc fragment. A CTLA-4-hFc lentiviral expression plasmid was produced by NheI-HF/BamHI-HF-directed cloning of the CTLA-4-hIgG₁ Fc fragment into pLenti-C-Myc/DDK-IRES-Puro (Origene), resulting in four pLenti-C-CTLA-4-hIgG₁ FC-IRES-Puro clones (denoted clones 1-4). This expression vector was then transfected by Lipofectamine 2000 (Life Technologies) transfection into human HEK293T. 48-hour post-transfection supernatants were collected prior to serial dilution and quantification using a proprietary ELISA test for Fc-fusion PantId production. FIG. 9 shows the titers of supernatant CTLA-4-hFc PantId obtained from each of the four lentiviral clones into human HEK293T cells. Additional titers from control samples are also shown in FIG. 9, including the following: two negative controls (i.e. diluted culture medium and the pLenti-C-Myc/DDK-IRES-Puro vector), which both gave the expected negative result for expression of the PantId. Also shown is the titer in supernatant from vLenti-C-CTLA-4-hIgG₁ Fc-IRES-Puro lentivirally transduced HEK293T cells, which provided modest expression compared with the transfected cells.

In other embodiments, any of the Pant-Ids described throughout this specification can be cloned, expressed, and characterized using this approach. For example, in some embodiments and optional features herein, the PantIds that are cloned and expressed comprise, for example, an immunological checkpoint receptor, immunological checkpoint ligand, and/or immunoregulatory cytokine selected from but not limited to; PD-1 (Sequence 038); CD28 (Sequence 039); CTLA-4 (Sequence 040); ICOS (Sequence 041); BTLA (Sequence 042); a killer immunoglobulin receptor (KIR), including: KIR2DL1 (Sequence 043), KIR2DL2 (Sequence 044), KIR2DL3 (Sequence 045), KIR2DL4 (Sequence 046), KIR2DL5A (Sequence 047), KIR2DL5B (Sequence 048), KIR2DS1 (Sequence 049), KIR2DS2 (Sequence 050), KIR2DS3 (Sequence 051), KIR2DS4 (Sequence 052), KIR2DS5 (Sequence 053), KIR3DL2 (Sequence 054), KIR3DL3 (Sequence 055), and KIR3DS1 (Sequence 056); LAG-3 (Sequence 057); CD137 (Sequence 058); OX40 (Sequence 059); CD27 (Sequence 060); CD40 (Sequence 061); TIM-3 (Sequence 062) and other T-cell immunoglobulin and 1-domain containing (TIM) receptors, including TIM-1 (Sequence 063), TIM-2 (Sequence 064), and TIM-4 (Sequence 065); A2aR (Sequence 066); or any transmembrane, peripheral membrane, membrane-associated, or cytosolic protein containing an ITAM (immunoreceptor tyrosine-based activating motif, Sequence 067), ITIM (immunoreceptor tyrosine-based inhibitory motif, Sequence 068), or ITSM (immunoreceptor tyrosine-based switch motif, Sequence 069) motif, domain, or peptide, such as CD244 (2B4, Sequence 070) and TIGIT receptor (Sequence 071).

In some embodiments and optional features, the PantId may comprise an immunological checkpoint receptor, immunological checkpoint ligand, and/or immunoregulatory cytokine selected from but not limited to; CTLA-4, PD-1, BTLA, LAG-3, TIM-3, LAIR, TIGIT, Siglec-2, Siglec-3, Siglec-4, Siglec-10, FcγRII, CD5, CD66a, PIR-B, ILT-2, and CD72.

In some embodiments, the effector component of the PantId cloned and expressed may be any effector described throughout this specification, and may be selected, for example, from any of the following, or its ligand, or may exclude any of the following; any protein, domain, peptide, glycan, lipid, nucleic acid, glycoprotein, lipoprotein, ribonucleoprotein, deoxyribonucleoprotein, covalently-modified peptide, or small-molecule of less than 10,000 Daltons, or combinations or molecular chimeras thereof, capable of inducing apoptosis, necrosis, cytostasis, tolerization, or anergy in leukocytes, optionally T and B cells. In some embodiments, the effector component of the PantId cloned, expressed and/or characterized herein can be selected from or may exclude any of the following or its binding partner: death receptor ligands, comprising CD95L (a.k.a. FasL, Sequence 001), TRAIL (a.k.a. Apo2L, Sequence 002), and TWEAK (a.k.a. Tumor necrosis factor ligand superfamily member 12, Sequence 003) of the effector class of PantIds. In some embodiments, the effector may include or exclude any other member of the TNF receptor superfamily ligands including, but not limited to, OX40L (Sequence 004), TNF-α (Sequence 005), Lymphotoxin-β (a.k.a. TNF-C, Sequence 006) and its binding partner Lymphotoxin-α (a.k.a. TNF-β, Sequence 007), CD154 (a.k.a. CD40L, Sequence 008), LIGHT (a.k.a. CD258 Sequence 009), CD70 (Sequence 010), CD153 (Sequence 011), 4-1BBL (a.k.a. CD137L, tumor necrosis factor (ligand) superfamily, member 9, (Sequence 012), RANKL (a.k.a. CD254, Sequence 013), APRIL (Sequence 014), Nerve growth factor ligands (e.g. NGF Sequence 015, BDNF (Sequence 016), NT-3 (Sequence 017), and NT-4 (Sequence 018), BAFF (Sequence 019), GITR ligand (Sequence 020), TL1A (Sequence 021), and EDA-A2 (Sequence 022), modified bacterial toxins, including A-B toxins and autotransporters, for the delivery of cytotoxic effectors intracellularly, wherein said cytotoxic effector may be a caspase, bacterial toxin, or other enzyme; a cytotoxic or cytostatic agent small-molecule of less than 10,000 Daltons, such as microtubule or actin cytoskeletal modulators, inhibitors of DNA replication, ribosomal inhibitors, inhibitors of RNA synthesis, radionuclides and coordination complexes thereof, etc.; an NK activating receptor ligand, including: MICA (Sequence 023) and MICB (Sequence 024), which bind NKG2D; ULBP1-6 (Sequences 025-030), Rae-1 (Sequence 031), MULTI (Sequence 032), H60 (Sequence 033), which bind to NKG2D; the DNAM-1 ligands, CD155 (Sequence 034) and CD112 (Sequence 035); B7-H6 (Sequence 036) and BAT3 (Sequence 037); which bind to NKp30; and CD27, which binds CD70; an immunomodulatory cytokine, such as IL-1β, IL-6, IL-7, IL-10, IL-12, IL-21, IL-35, TGF-β, TNF-α, type I interferons, type II interferons, type III interferons, canonical chemokines (e.g. CC, CXC, C, and CX₃C classes), and non-canonical chemotactic or chemokinetic agents (e.g. Slit1, 2, and 3); or an Fc domain of human, murine, porcine, or canine immunoglobulins, including IgA, IgM, IgG, IgD, IgE, and their subclasses. In some embodiments the Fc can increase the bioavailability and/or half-life of the PantId. In some embodiments the PantId effector component may exclude any of the Fc domains listed above.

### Example 8—Demonstration of Oligonmeric/Homodimeric Structure of a PantId

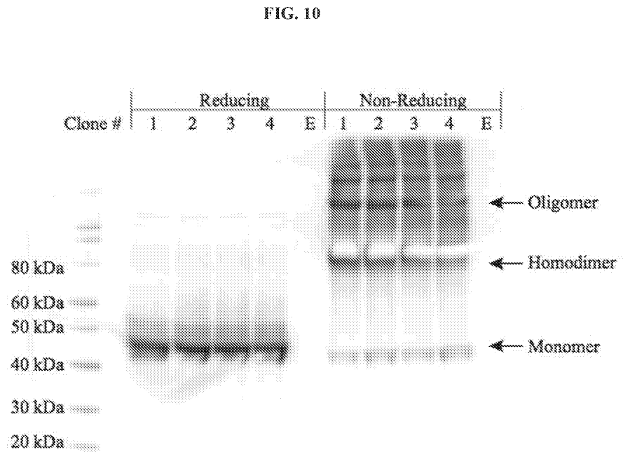

The oligonmeric/homodimeric structure of the CTLA-4-hFc PantId was determined to be homodimeric, as expected. The structure and the size of the CTLA-4-hFc PantId were confirmed by Western Blot analysis. CTLA-4-hFc, along with pLenti-C-CTLA-4-hIgG1 FC-IRES-Puro clones 1-4 were transfected into HEK293T cells and the supernatants were analyzed in the presence or absence of a reducing agent. This allowed identification of the monomers, homodimers, and higher order oligomers. Clone numbers are indicated by numerals, and the empty parental pLenti-C-Myc/DDK-IRES-Puro vector was used as a control. The proper homodimeric form is a predominant band in non-reduced samples, indicating appropriate structure. Additionally, in the reduced samples, the CTLA-4-hFc monomer exhibits the predicted molecular mass of 43 kDa. Higher molecular weight bands correspond to oligomers and glycovariants thereof. The results are shown in FIG. 10.

### Example 9—First Components of PantIds Binding to Anti-Human CTLA-4, PD-1, and PD-L1 Antibodies

Purified CTLA-4-Fc, PD-1-CCAN4, and PD-L1-CCAN4 first components of PantIds were prepared in LDS sample buffer and heated at 80° C. prior to loading on a Bis-Tris SDS-PAGE gel alongside a marker ladder. Following electrophoresis, the polypeptides were transferred electrophoretically to nitrocellulose membranes. The nitrocellulose membranes were blocked in Tris-buffered saline (TBS) with 0.1% Tween 20 and 5% skim milk 5% skim milk before staining with 1 μg/ml of mouse anti-human CTLA-4 (Abcam catalog number: ab177523), mouse anti-human PD-1 (Abcam catalog number: ab52587), or rabbit anti-human PD-L1 (ProSci catalog number: 4059) overnight at 4° C. in TBS-T with 5% skim milk. A control membrane which received only secondary staining, was left in blocking reagent overnight. The following day, after washing three times in TBS-T, the membranes were stained with a 1:4,000 dilution of goat anti-mouse, HRP conjugate (Thermo Fisher Catalog Number: A16078) or goat anti-rabbit, HRP conjugate (Jackson ImmunoResearch Catalog Number: 111-035-003) in TBS-T with 5% skim milk for 1 hour. Membranes were washed three times prior to ECL development with SuperSignal™ West Femto Maximum Sensitivity Substrate: (Thermo Fisher Catalog Number: 34096) and imaged on an Azure Biosystems imaging station. The results are shown in FIG. 11. As shown in FIG. 11, anti-CTLA-4 antibody specifically bound to the CTLA-4-Fc first component of a PantId (left-hand panel). The control membrane, which was exposed only to anti-mouse IgG secondary antibody is shown in the adjacent left-hand center panel. Little or no nonspecific binding was observed in a 30 second exposure. As also shown in FIG. 11, anti-PD-1 and anti-PD-L1 antibodies specifically bound PD-1-CCAN4, and PD-L1-CCAN4 first components of a PantId, respectively (see the right-hand center panel and the far right hand panel.)

### Example 10—Neutralization Anti-PD-1 Antibody by PD-1-CCAN4 First Component of a PantId In Vitro

Recombinant human PD-1 protein (Abcam catalog number: 174035) was reconstituted in PBS to 0.5 mg/ml. This stock was diluted 500-fold in BupH Carbonate/Bicarbonate ELISA coating buffer to generate the 1 μg/ml recombinant PD-1 working reagent, of which 100 μl (100 ng of recombinant PD-1) was added to each well of an ELISA plate. After coating overnight at 4° C., the plate was washed three times with PBS with 0.05% Tween 20, and then blocked with PBS with 5% skim milk for two hours at room temperature. During this time, a 1 μg/ml solution of mouse anti-human PD-1 (Abcam catalog number: ab52587) was prepared in PBS. 1 μg of PD-1-CCAN4 first component of a PantId, 1 μg of human IgG negative control, and serial two-fold dilutions thereof were mixed with the anti-PD-1 antibody for neutralization over the course of one hour at room temperature. Thereafter, the plate was washed, and the neutralized antibody mixes were added to their appropriate well for binding for 1 hour at room temperature. Plates were subsequently washed and then stained with goat anti-mouse, HRP conjugate (Thermo Fisher Catalog Number: A16078) for one hour at room temperature before another wash. TMB substrate (Thermo Fisher Catalog Number: 34028) was added to each well until chromatophore development was apparent, after which the reaction was stopped with 2N H₂SO₄. Plates were read at 450 nm on a Beckman Coulter DTX multimode detector.

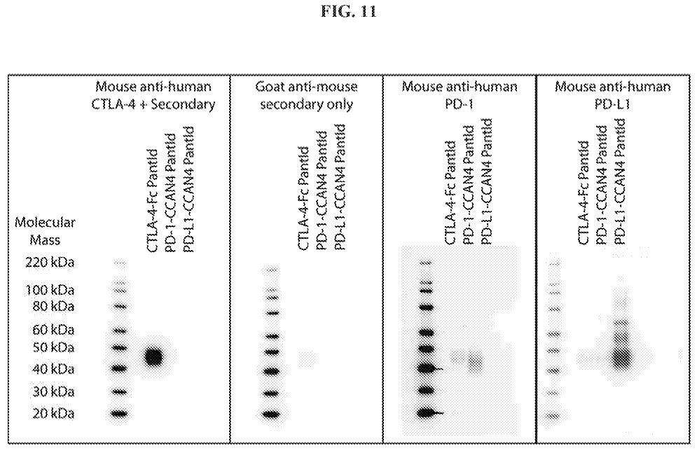

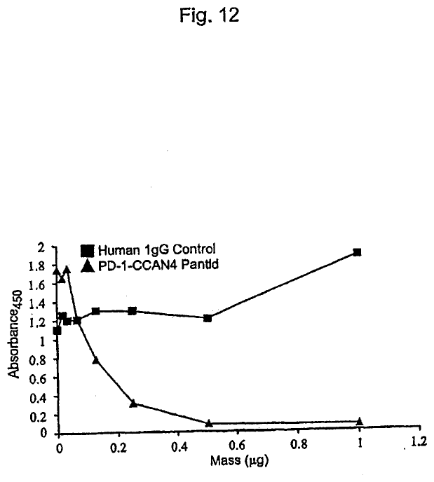

As shown in FIG. 12, PD-1-CCAN4 first component of a PantId specifically neutralized the binding of mouse anti-human PD-1 to recombinant human PD-1 protein. The neutralization activity was dose-dependent and was not observed for the human IgG control antibody.

### Example 11—Neutralization of Anti-PD-1 Antibody by PD-1-CCAN4 First Component of a PantId In Vitro

Recombinant human PD-1 protein (Abcam catalog number: 174035) was reconstituted in PBS to 0.5 mg/ml. This stock was diluted 500-fold in BupH Carbonate/Bicarbonate ELISA coating buffer to generate the 1 μg/ml recombinant PD-1 working reagent, of which 100 μl (100 ng of recombinant PD-1) was added to each well of an ELISA plate. After coating overnight at 4° C., the plate was washed three times with PBS with 0.05% Tween 20, and then blocked with PBS with 5% skim milk for two hours at room temperature. During this time, a 1 μg/ml solution of mouse anti-human PD-1 (Abcam catalog number: ab52587) was prepared in PBS. 2 μg of PD-1-CCAN4 of a first component of a PantId, 2 μg of human IgG negative control, and 2 μg of BSA negative control, and serial two-fold dilutions thereof were mixed with the anti-PD-1 antibody for neutralization over the course of one hour at room temperature. Thereafter, the plate was washed, and the neutralized antibody mixes were added to their appropriate well for binding for 1 hour at room temperature. Plates were subsequently washed and then stained with goat anti-mouse, HRP conjugate (Thermo Fisher Catalog Number: A16078) for one hour at room temperature before another wash. TMB substrate (Thermo Fisher Catalog Number: 34028) was added to each well until chromatophore development was apparent, after which the reaction was stopped with 2N H₂SO₄. Plates were read at 450 nm on a Beckman Coulter DTX multimode detector. Mass, in μg, was log-transformed for further analysis.

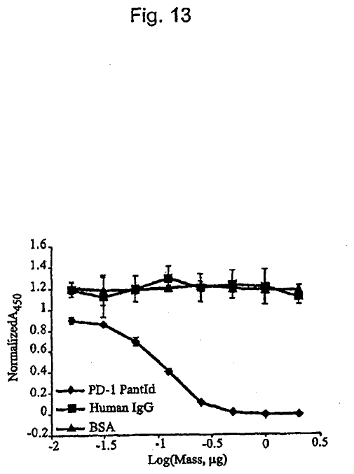

As shown in FIG. 13, PD-1-CCAN4 first component of a PantId specifically neutralized the binding of mouse anti-human PD-1 to recombinant human PD-1 protein. The neutralization activity was dose-dependent and was not observed for the samples which contained human IgG control antibody or BSA. PD-1-CCAN4 first component of a PantId neutralized 1 μg/ml anti-human PD-1 with an IC₅₀ of 136 ng or 31.8 nM, with PD-1-CCAN4 first component of a PantId exhibiting an observed molecular weight in SDS-PAGE of 43 kDa.

### Example 12—Binding of CTLA-4-Fc First Component of a PantId to Anti-Human CTLA-4 Monoclonal Antibody

Samples containing 840 ng of either reduced or non-reduced CTLA-4-Fc first component of a PantId were analyzed on a 4-12% Bis-Tris polyacrylamide gel. For reduction, samples were treated with SDS sample buffer containing beta-mercaptoethanol. The gel was stained 1 μg/ml anti-human CTLA-4 (Abcam catalog number ab177523) in Tris-buffered saline (TBS) with 0.1% Tween-20 and 5% skim milk overnight. After washing, the gel was stained with goat anti-mouse IgG (H+L) HRP-conjugate (Thermo Fisher Catalog Number: A16066) as a 1:4,000 dilution in TBS-T with 5% skim milk. Chemiluminescence was generated using SuperSignal West Femto Maximum Sensitivity Substrate.

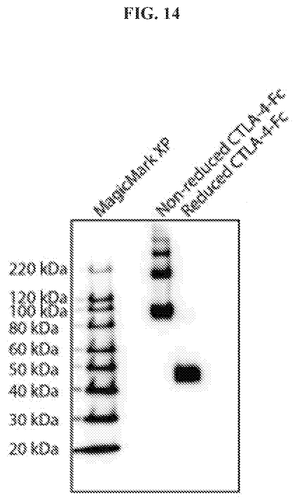

As shown in FIG. 14, CTLA-4-Fc PantId was specifically bound by anti-human CTLA-4. Binding was observed for both non-reduced and reduced CTLA-4-Fc PantId.

### Example 13—Purification of PD-L1-CCAN4-SBP Polypeptide by Strep-Tactin Resin

A lentiviral expression vector encoding the PD-L1 extracellular domain fused to the CCAN4 heterodimerization domain and the Strep Tag II streptavidin-binding peptide (SBP), pLenti-PD-L1-CCAN4-SBP, was transfected into HEK293T cells. Supernatant (2 ml) was harvested and subjected to purification using Strep-Tactin resin (QIAGEN Catalog Number: 30002). Fractions were analyzed on an SDS-PAGE gel. Polypeptides were visualized by Coomassie Blue staining.

As shown in FIG. 15, PD-L1-CCAN4-SBP polypeptide (“PD-L1 heterodimeric PantId”) was recovered from the Strep-Tactin Resin in the first and second elution fractions.

### Example 14—Purification of PD-L1-CCAN4-SBP Polypeptide by Strep-Tactin Resin and FasL and TRAIL Heterodimeric Second Components of a PantId Expression in CHO Cells

A lentiviral expression vector encoding the PD-L1 extracellular domain fused to the CCAN4 heterodimerization domain and the Strep Tag II streptavidin-binding peptide (SBP), pLenti-PD-L1-CCAN4-SBP, was transfected into HEK293T cells. Supernatant (2 ml) was harvested and subjected to purification using Strep-Tactin resin (QIAGEN Catalog Number: 30002). pLenti-PD-1-CCAN4-SBP, a lentiviral expression vector encoding the PD-1 extracellular domain fused to the CCAN4 heterodimerization domain and the Strep Tag II streptavidin-binding peptide (SBP), was transfected into HEK293T cells. 2 ml of supernatant was harvested and subjected to purification using Strep-Tactin resin (QIAGEN Catalog Number: 30002). Fractions were run on an SDS-PAGE gel prior to immunoblot using anti-Strep Tag II antibody-HRP conjugate (EMD Milipore Catalog Number: 71591-3). Similarly, CHO cells were transfected with pLent-FasL-CCBN4-SBP and pLenti-TRAIL-CCBN4-SBP. pLent-FasL-CCBN4-SBP expressed FasL fused to the cognate CCBN4 heterodimerization domain and Strep Tag II SBP. pLenti-TRAIL-CCBN4-SBP expressed theTRAIL extracellular domain fused to the cognate CCBN4 heterodimerization domain and Strep Tag II SBP. Pellets and supernatants were harvested and analyzed by SDS-PAGE and and immunoblotting with anti-Strep Tag II.

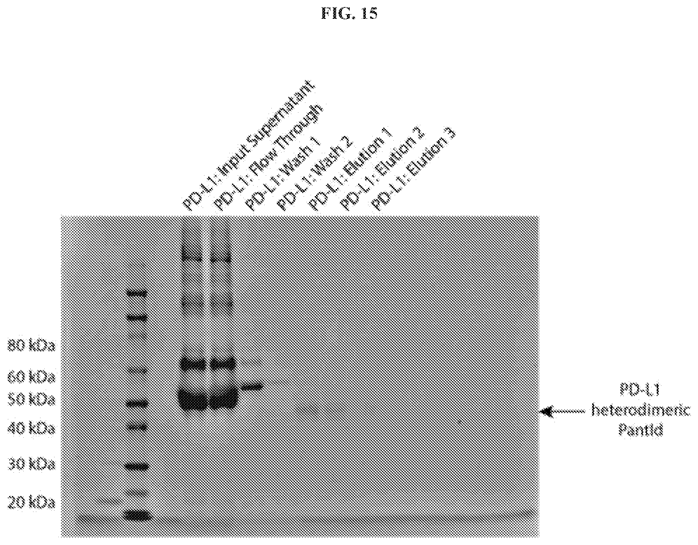

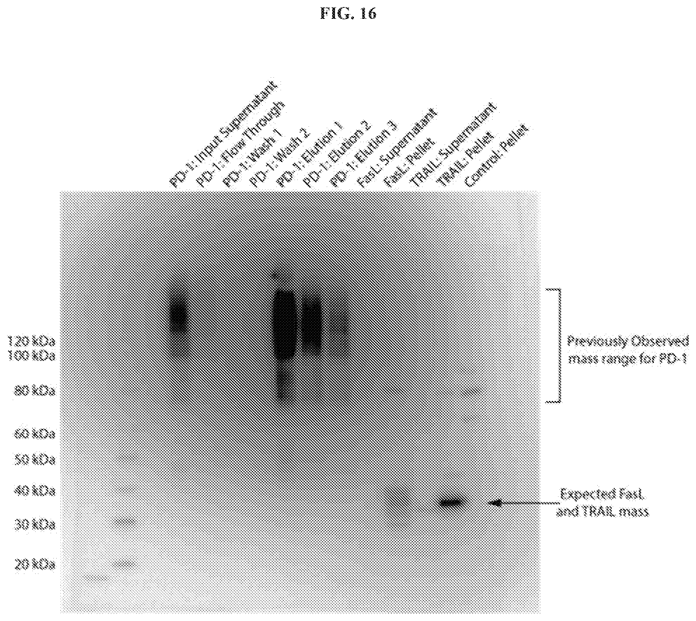

As shown in FIG. 16, PD-L1-CCAN4-SBP polypeptide (“PD-L1 heterodimeric PantId”) was recovered from the Strep-Tactin Resin in the first and second elution fractions. As also shown in FIG. 16, CHO cells expressing FasL-CCBN4-SBP or TRAIL-CCBN4-SBP produce polypeptides of the expected mass.

The inventions described and claimed herein have many attributes and embodiments including, but not limited to, those set forth or described or referenced in this disclosure. It is not intended to be all-inclusive and the inventions described and claimed herein are not limited to or by the features or embodiments identified in this disclosure, which is included for purposes of illustration only and not restriction. A person having ordinary skill in the art will readily recognize that many of the components and parameters may be varied or modified to a certain extent or substituted for known equivalents without departing from the scope of the invention. It should be appreciated that such modifications and equivalents are herein incorporated as if individually set forth. The invention also includes all of the steps, features, compositions and compounds referred to or indicated in this specification, individually or collectively, and any and all combinations of any two or more of said steps or features.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents. Reference to any applications, patents and publications in this specification is not, and should not be taken as, an acknowledgment or any form of suggestion that they constitute valid prior art or form part of the common general knowledge in any country in the world.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a”, “an”, and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants. Furthermore, titles, headings, or the like are provided to enhance the reader's comprehension of this document, and should not be read as limiting the scope of the present invention. Any examples of aspects, embodiments or components of the invention referred to herein are to be considered non-limiting.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

All publications and patent applications mentioned in this specification are incorporated by reference herein to the same extent as if each individual publication or patent application was specifically and individually indicated to be incorporated by reference.

### REFERENCES CITED IN THE DISCLOSURE

1. Sojka, D. K., Huang, Y.-H. & Fowell, D. J. Mechanisms of regulatory T-cell suppression—a diverse arsenal for a moving target. Immunology 124, 13-22 (2008).
2. Jones, A. et al. Immunomodulatory Functions of BTLA and HVEM Govern Induction of Extrathymic Regulatory T Cells and Tolerance by Dendritic Cells. Immunity 45, 1066-1077 (2016).
3. Ercolini, A. M. & Miller, S. D. The role of infections in autoimmune disease. Clin. Exp. Immunol. 155, 1-15 (2009).
4. Faé, K. C. et al. How an autoimmune reaction triggered by molecular mimicry between streptococcal M protein and cardiac tissue proteins leads to heart lesions in rheumatic heart disease. J. Autoimmun. 24, 101-109 (2005).
5. Root-Bernstein, R. Rethinking Molecular Mimicry in Rheumatic Heart Disease and Autoimmune Myocarditis: Laminin, Collagen IV, CAR, and B1AR as Initial Targets of Disease. Front. Pediatr. 2, (2014).
6. Michot, J. M. et al. Immune-related adverse events with immune checkpoint blockade: a comprehensive review. Eur. J. Cancer 54, 139-148 (2016).
7. Munir, S. et al. HLA-Restricted CTL That Are Specific for the Immune Checkpoint Ligand PD-L1 Occur with High Frequency in Cancer Patients. Cancer Res. 73, 1764-1776 (2013).
8. Munir, S., Andersen, G. H., Svane, I. M. & Andersen, M. H. The immune checkpoint regulator PD-L1 is a specific target for naturally occurring CD4+ T cells. Oncoimmunology 2, (2013).
9. Matsui, T. et al. Autoantibodies to T cell costimulatory molecules in systemic autoimmune diseases. J. Immunol. Baltim. Md. 1950 162, 4328-4335 (1999).
10. Matsui, T., Nishioka, K., Kato, T. & Yamamoto, K. Autoantibodies to CTLA-4 enhance T cell proliferation. J. Rheumatol. 28, 220-221 (2001).
11. Thomas, F., Boyle, A. L., Burton, A. J. & Woolfson, D. N. A Set of de Novo Designed Parallel Heterodimeric Coiled Coils with Quantified Dissociation Constants in the Micromolar to Sub-nanomolar Regime. J. Am. Chem. Soc. 135, 5161-5166 (2013).
12. Mittl, P. R. E. et al. The retro-GCN4 leucine zipper sequence forms a stable three-dimensional structure. Proc. Natl. Acad. Sci. 97, 2562-2566 (2000).
13. Cho, A., Haruyama, N. & Kulkarni, A. B. Generation of Transgenic Mice. Curr. Protoc. Cell Biol. Editor. Board Juan Bonifacino Al CHAPTER, Unit-19.11 (2009).

1. A PantId molecule for treatment of autoimmune diseases or disorders in which autoreactive B cells respond to immunological checkpoint receptors, immunological checkpoint ligands, and/or immunoregulatory cytokines, said molecule comprising: a molecular chimera of two, three, four, or five proteins, domains, or peptides:
wherein a first of the proteins, domains, or peptides is one of an immunological checkpoint receptor, a checkpoint ligand, or an immunoregulatory cytokine; and
wherein a second of the proteins, domains, or peptides is an effector.

2.-72. (canceled)
